(19) United States
(12) Patent Application Publication
(10) Pub. No.: US 2020/0155437 A1
(43) Pub. Date: May 21, 2020

(54) Personal Care Composition

(71) Applicant: The Procter & Gamble Company, Cincinnati, OH (US)

(72) Inventor: Juan Esteban Velasquez, Cincinnati, OH (US)

(21) Appl. No.: 16/751,577

(22) Filed: Jan. 24, 2020

Related U.S. Application Data
(63) Continuation of application No. PCT/US2018/060696, filed on Nov. 13, 2018.
(60) Provisional application No. 62/585,133, filed on Nov. 13, 2017.

(51) Int. Cl. A61K 8/64; C07K 14/32; A61K 8/73; A61Q 19/00

(52) U.S. Cl. CPC A61K 8/64; C07K 14/32; A61K 2800/596; A61Q 19/00; A61K 2800/10; A61K 8/73

(57) ABSTRACT

A personal care composition is disclosed. The composition comprises: a) one or more surface active proteins selected from the group consisting of Class III BslA proteins, Class IV BslA proteins, chaplin proteins, ranaspumins, latherins, and mixtures thereof and b) a surfactant.

### FIELD OF THE INVENTION

The present invention relates to a personal care composition comprising one or more surface active proteins and a surfactant. The composition can provide one or more benefits, including good cleaning, good grease emulsification, long lasting suds, and surface modification that can contribute to second time cleaning benefits, improved drying, and improved shine.

### BACKGROUND OF THE INVENTION

Personal care compositions should provide good cleaning benefits while presenting a good suds profile in particular a long lasting suds profile especially in the presence of greasy soils. Users usually see suds as an indicator of the performance of the personal care compositions. Moreover, the user of a personal care composition may also use the suds profile and the appearance of the suds (e.g., density, whiteness) as an indicator that such composition contains active cleaning ingredients. Accordingly, it is desirable for a personal care composition to provide “good sudsing profile”, which includes good suds height and/or density as well as good suds duration during the initial mixing of the composition with water and/or during the entire cleaning operation.

Several families of natural surface active proteins are able to aid suds performance in aqueous solutions (see Cooper, A., et al. (2017), Colloids Surf., A: Physiochemical and Engineering Aspects; Schor, M., et al. (2016), Trends Biochem. Sci. 41(7): 610-620). In particular, the surface active Class I or Class II BslA (Biofilm surface layer A) proteins have been used as a stabilizer in synthetic multiphase products that include sudsing agents to prevent phase separation and improve the sudsing performance of the products in liquid during use (see US2017/267730 (University of Edinburgh)). However, the amount of sudsing generated by such class I or class II BslA proteins in personal care compositions is limited. This challenge cannot be solved by simply increasing the Class I or Class II BslA concentration level in the composition. That is because while the Class I or Class II BslA proteins may perform well in isolation, their performance may degrade (noticeably) in the presence of surfactants that are typically present in personal care compositions. In another example related to cosmetic compositions, the use of surface active hydrophobin proteins for treatment or delivery of active ingredients has been also described in the art (US2009/0136433A1, US 2003/0217419A1).

Accordingly, the need remains for an improved personal care composition comprising surface active proteins which has a further improved sudsing profile, particularly at low surface active proteins concentrations in the personal care compositions. The need also exists for an improved personal care composition that provides a pleasant washing experience, e.g., good feel on the user's hands during the wash. The composition should also be easy to rinse. Further it is desirous that the improved personal care composition is stable and will not phase separate, resulting in greater shelf-life of the product. It is also desirable that personal care compositions provide surface modification, contributing to shine (e.g., in the case of hair), improved second time cleaning. There is also the desire to reduce the amount of surfactants without negatively impacting sudsing nor grease cleaning and emulsification profile. Thus, there is the need to find new compositions that improve cleaning, suds longevity and improved after cleaning benefits under personal care cleaning conditions. The Applicant discovered that some or all of the above-mentioned needs can be at least partially fulfilled through the improved personal care compositions as described herein below.

### SUMMARY OF THE INVENTION

A personal care composition is disclosed. The composition comprises: a) one or more surface active proteins selected from the group consisting of Class III BslA proteins, Class IV BslA proteins, chaplin proteins, ranaspumins, latherins, and mixtures thereof and b) a surfactant. In a certain embodiment, the Class III BslA protein has at least 50%, preferably at least 60%, preferably at least 70%, preferably at least 80%, preferably at least 85%, preferably at least 90%, preferably at least 95%, preferably at least 98% or even 100% amino acid identity to a wild-type protein: Thermoactinomyces vulgaris BslA (SEQ ID NO: 6), and the Class IV BslA protein has at least 50%, preferably at least 60%, preferably at least 70%, preferably at least 80%, preferably at least 85%, preferably at least 90%, preferably at least 95%, preferably at least 98% or even 100% amino acid identity to at least one wild-type protein sequence selected from the group consisting of: B. licheniformis BslA (SEQ ID NO: 7, SEQ ID NO: 8, SEQ ID NO: 9, SEQ ID NO: 10, and SEQ ID NO: 11), B. glycinifermentans BslA (SEQ ID NO: 12, SEQ ID NO: 13, SEQ ID NO: 14, and SEQ ID NO: 15), B. sonorensis BslA (SEQ ID NO: 16), B. paralicheniformis BslA (SEQ ID NO: 17, and SEQ ID NO: 18), and Bacillus sp. BslA (SEQ ID NO: 19, SEQ ID NO: 20, SEQ ID NO: 21, and SEQ ID NO: 22). Preferably, the Class IV BslA protein has at least 90%, preferably at least 95%, preferably at least 98% or even 100% amino acid identity to B. licheniformis BslA (SEQ ID NO: 7).

In another embodiment, the chaplin proteins have at least 50%, preferably at least 60%, preferably at least 70%, preferably at least 80%, preferably at least 85%, preferably at least 90%, preferably at least 95%, preferably at least 98% or even 100% amino acid identity to at least one protein selected from the group consisting of SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, or SEQ ID NO: 55, preferably to at least one wild-type protein selected from the group consisting of: Streptomyces coelicolor ChpD (SEQ ID NO: 48), Streptomyces coelicolor ChpE (SEQ ID NO: 49), Streptomyces coelicolor ChpF (SEQ ID NO: 50), Streptomyces coelicolor ChpG (SEQ ID NO: 51), and Streptomyces coelicolor ChpH (SEQ ID NO: 52), more preferably Streptomyces coelicolor ChpE (SEQ ID NO: 49) and Streptomyces coelicolor ChpF (SEQ ID NO: 50).

In a certain embodiment, the ranaspumins have at least 40%, preferably at least 50%, preferably at least 60%, preferably at least 70%, preferably at least 80%, preferably at least 85%, preferably at least 90%, preferably at least 95%, preferably at least 98% or even 100% amino acid identity to at least one wild-type protein selected from the group consisting of: Engystomops pustulosus Ep-Rsn2 (SEQ ID NO: 56), Leptodactylus vastus Lv-Rsn1 (SEQ ID NO: 57), Leptodactylus fuscus Lf-Rsn1 (SEQ ID NO: 58), and Bufo gargarizans Bg-Rsn (SEQ ID NO: 59), preferably Engystomops pustulosus Ep-Rsn2 (SEQ ID NO: 56) and Leptodactylus vastus Lv-Rsn1 (SEQ ID NO: 57).

In another embodiment, the composition further comprises one or more co-proteins, wherein the co-proteins have at least 40%, preferably at least 50%, preferably at least 60%, preferably at least 70%, preferably at least 80%, preferably at least 85%, preferably at least 90%, preferably at least 95%, preferably at least 98% or even 100% amino acid identity to at least one wild-type protein selected from the group consisting of: Engystomops pustulosus Ep-Rsn1 (SEQ ID NO: 60), Engystomops pustulosus Ep-Rsn3 (SEQ ID NO: 61), Engystomops pustulosus Ep-Rsn4 (SEQ ID NO: 62), Engystomops pustulosus Ep-Rsn5 (SEQ ID NO: 63), and Engystomops pustulosus Ep-Rsn6 (SEQ ID NO: 64); and mixtures thereof, preferably Engystomops pustulosus Ep-Rsn3 (SEQ ID NO: 61) and Engystomops pustulosus Ep-Rsn5 (SEQ ID NO: 63). In a certain embodiment, the composition further comprises one or more carbohydrates selected from the group consisting of 0-glycan, N-glycan, and mixtures thereof.

In one embodiment, the latherins have at least 40%, preferably at least 50%, preferably at least 60%, preferably at least 70%, preferably at least 80%, preferably at least 85%, preferably at least 90%, preferably at least 95%, preferably at least 98% or even 100% amino acid identity to Equus caballus latherin (SEQ ID NO: 65). In a certain embodiment, the surface active proteins are present in an amount from 0.0001 wt % to 5 wt %, preferably from 0.01 wt % to 1 wt %, by weight of said composition based on active protein. In another embodiment, the surfactant is present in an amount from 2 wt % to 30 wt %, preferably from 3 wt % to 25 wt %, by weight of the composition.

### BRIEF DESCRIPTION OF THE DRAWINGS

While the specification concludes with claims particularly pointing out and distinctly claiming the invention, it is believed that the invention will be better understood from the following description of the accompanying figures:

FIG. 1 is a phylogenetic tree of BslA proteins identifying four different classes. The tree was generated using NCBI BLASTp (https://blast.ncbi.nbn.nih.gov/Blast.cgi) and manipulated with MEGA6 Ver. 6.06 software.

FIG. 2 is a sequence similarity network of BslA proteins identifying the four different classes. The network was generated using EFI—Enzyme Similarity Tool Ver 2.0 (https://efi.igb.illinois.edu/efi-est/).

FIG. 3 is a phylogenetic tree of Class I BslA (YuaB-like) proteins, expanded from FIG. 1.

FIG. 4 is a phylogenetic tree of Class II BslA (YweA-like) proteins, expanded from FIG. 1.

FIG. 5 is a phylogenetic tree of Class IV BslA proteins, expanded from FIG. 1.

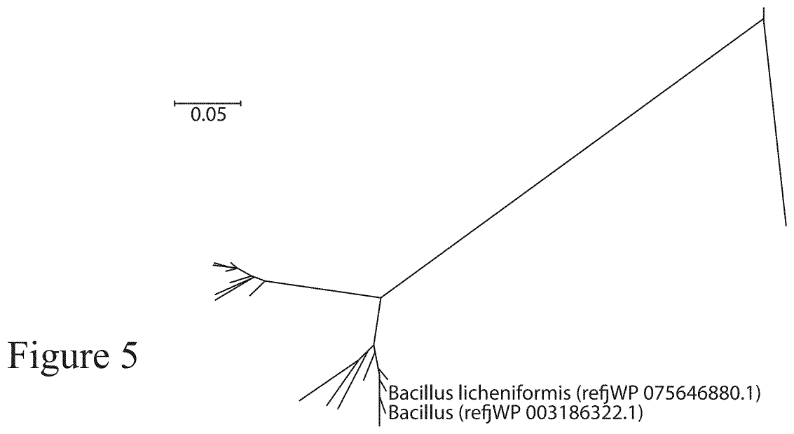

### DETAILED DESCRIPTION OF THE INVENTION

### I. Definitions

As used herein, the articles “a” and “an” when used in a claim, are understood to mean one or more of what is claimed or described.

As used herein, the term “substantially free of” or “substantially free from” means that the indicated material is present in an amount of no more than 5 wt %, preferably no more than 2%, and more preferably no more than 1 wt % by weight of the composition.

As used therein, the term “essentially free of” or “essentially free from” means that the indicated material is present in an amount of no more than 0.1 wt % by weight of the composition, or preferably not present at an analytically detectible level in such composition. It may include compositions in which the indicated material is present only as an impurity of one or more of the materials deliberately added to such compositions.

As used herein, the term “amino acid identity” means the identity between two or more amino acid sequences and is expressed in terms of the identity or similarity between the sequences. Sequence identity can be measured in terms of percentage identity; the higher the percentage, the more identical the sequences are. The percentage identity is calculated over the length of comparison. Methods of alignment of sequences for comparison are well known in the art and identity can be calculated by many known methods. Various programs and alignment algorithms are described in the art. It should be noted that the terms ‘sequence identity’ and ‘sequence similarity’ can be used interchangeably.

As used herein, the term “surface active proteins” refers to the wild-type surface proteins selected from the group consisting of BslA proteins, ranaspumins, latherins, chaplin proteins, hydrophobins, and mixtures thereof, and variants thereof. The BslA proteins within the scope of the present invention are Class III or Class IV BslA proteins. The hydrophobins within the scope of the present invention are Class II hydrophobins.

As used herein, the term “personal care composition” refers to compositions intended for topical application to skin and/or hair. Personal care compositions can be rinse-off formulations, in which the product can be applied topically to the skin and/or hair and then subsequently rinsed within seconds to minutes from the skin or hair with water. The product could also be wiped off using a substrate. The personal care compositions can also be used as shaving aids. The personal care compositions can be extrudable or dispensable from a package. Examples of personal care compositions can include but are not limited to bar soap, shampoo, conditioning shampoo, body wash, moisturizing body wash, shower gels, skin cleansers, cleansing milks, in shower body moisturizer, pet shampoo, shaving preparations, and cleansing compositions used in conjunction with a disposable cleansing cloth.

As used herein the term “fragment” means an amino acid sequence of at least 20, 40, 60, 80, 100, 150 contiguous amino acids of the reference sequences or any integer there between.

As used herein the term “increased suds longevity” means an increase in the duration of visible suds in a the personal care cleaning process the composition comprising one or more surface active proteins, compared with the suds longevity provided by the same composition and process in the absence of the surface active proteins.

As used herein, the term “next time cleaning benefit” means the surface to be cleaned (e.g., hair, skin, or teeth) could be treated with a composition which would assist in easier removal of soil during subsequent cleaning.

As used herein, the term “variant” of the surface active proteins means an amino acid sequence when the surface active protein is modified by, or at, one or more amino acids (for example 1, 2, 3, 4, 5, 6, 7, 8, 9 or 10 or more amino acid modifications) selected from substitutions, insertions, deletions and combinations thereof. The variant may have “conservative” substitutions, wherein a substituted amino acid has similar structural or chemical properties to the amino acid that replaces it, for example, replacement of leucine with isoleucine. A variant may have “non-conservative” changes, for example, replacement of a glycine with a tryptophan. Variants may also include sequences with amino acid deletions or insertions, or both. Guidance in determining which amino acid residues may be substituted, inserted, or deleted without abolishing the activity of the protein may be found using computer programs well known in the art. Variants may also include truncated forms derived from a wild type surface active protein, such as for example, a protein with a truncated N-terminus. Variants may also include forms derived by adding an extra amino acid sequence to a wild-type protein, such as for example, an N-terminal tag, a C-terminal tag or an insertion in the middle of the wild-type protein sequence.

As used herein, the term “water hardness” or “hardness” means uncomplexed cation ions (i.e., Ca²⁺ or Mg²⁺) present in water that have the potential to precipitate with anionic surfactants or other anionic actives in the personal care composition under alkaline conditions, and thereby diminishing the surfactancy and cleaning capacity of surfactants. Further, the terms “high water hardness” and “elevated water hardness” can be used interchangeably and are relative terms for the purposes of the present invention, and are intended to include, but not limited to, a hardness level containing at least 12 grams of calcium ion per gallon water (gpg, “American grain hardness” units).

### II. BslA Proteins

The personal care composition in accordance with the present invention can comprise one or more Class III or Class IV BslA proteins. Although BslA proteins have been referred to in the art as “bacterial hydrophobins”, they have very little sequence or structural similarity to the well-characterized fungal hydrophobins (Linder, M. B. (2009), Curr. Opin. Colloid Interface Sci. 14(5): 356-363.), which are not part of the current invention.

BslA proteins exhibit structural and functional similarity to Bacillus subtilis YuaB, a protein previously identified and reported in the art (Kobayashi, K. and M. Iwano (2012), Mol. Microbiol. 85(1): 51-66.). BslA proteins contain an unusually large hydrophobic cap on the surface, which is essential for their activity in the formation of hydrophobic, non-wetting biofilms. They usually participate in biofilm assembly, forming surface layers around such biofilms.

A number of proteins from several bacterial classes, including Clostridia, Bacteroidia Actinobacteria, and Chlorobia, appear to be related to B. subtilis YuaB, but either do not conserve the hydrophobic cap or contain additional protein domains. Thus, these proteins are not expected to have functional similarity to B. subtilis YuaB. In the context of the current invention, proteins with sequence similarity to YuaB but with no hydrophobic cap or with additional protein domains are not classified as “BslA proteins”.

The wild-type B. subtilis YuaB adopts a first conformation that is soluble in water, which transitions to a second conformation when adsorbed at an interface to expose hydrophobic residues to form the hydrophobic cap. This hydrophobic cap anchors YuaB protein at the interface between the phases by extending into the non-aqueous or non-polar phase. In addition, YuaB in the second configuration self-assembles to form a highly structured two-dimensional lattice at the interface. This two-dimensional lattice forms a viscoelastic film at the interface, which increases the stability of the interface, and resists rearrangement or relaxation of the interface after compression or deformation. Certain variants of wild-type YuaB, such as the L77K variant, do not retain the same ability as YuaB to form the highly structured two dimensional lattice at the interface, presumably as the mutation destabilizes the hydrophobic cap; it has significant interfacial activity, but does not form the same large-scale two-dimensional lattice as observed with the wild-type YuaB protein in which the hydrophobic cap is unaltered.

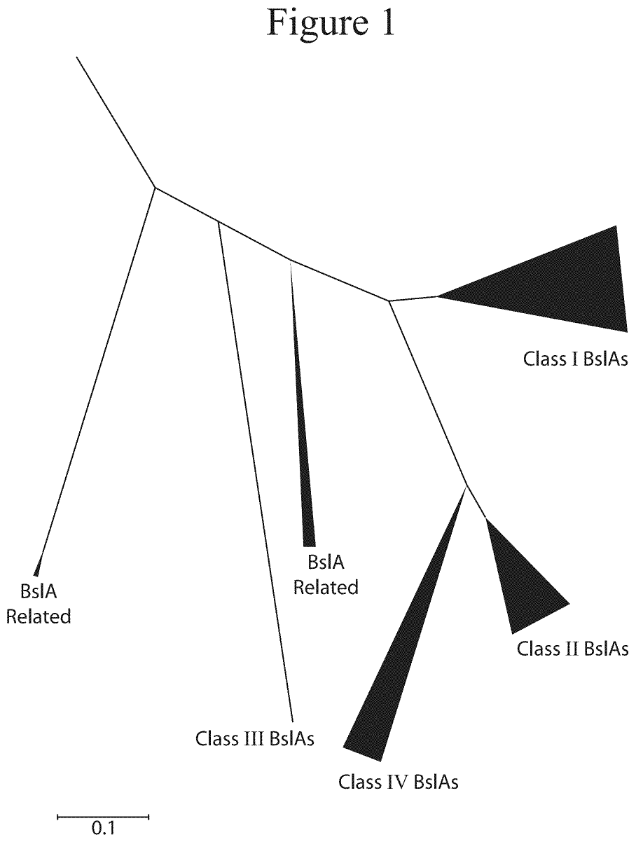

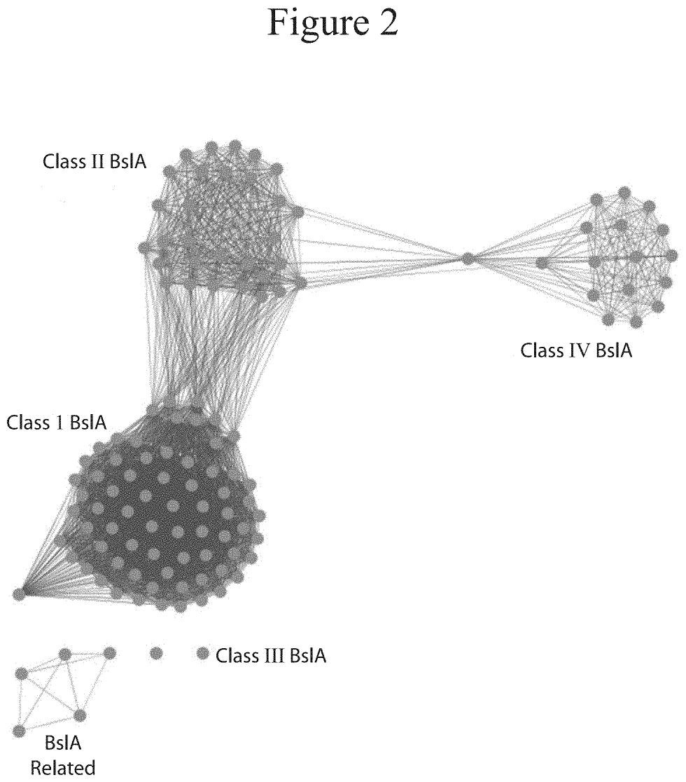

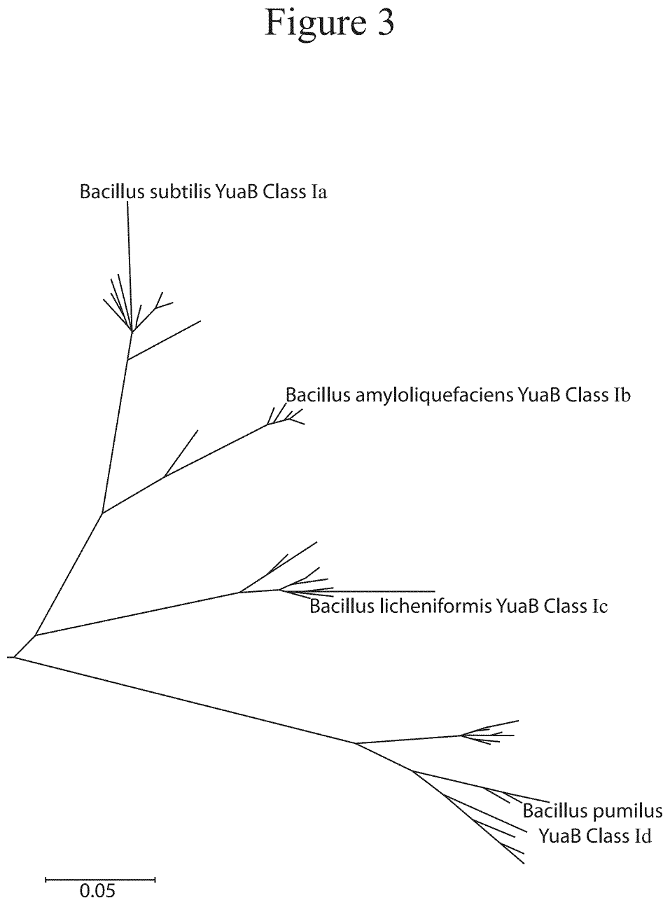

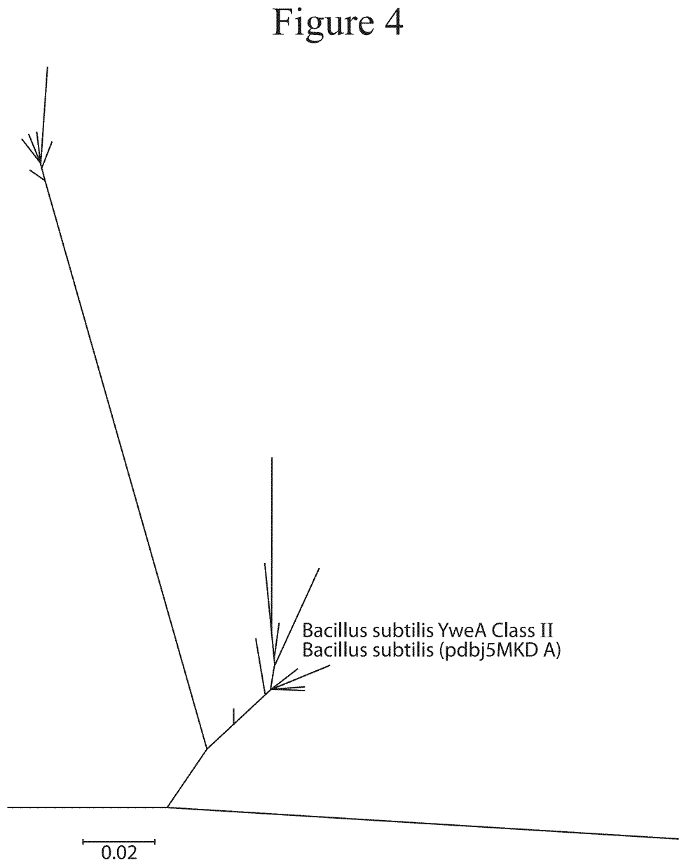

All BslA proteins with hydrophobic caps that have been reported in the art are from the genus Bacillus. For example, B. subtilis YuaB (SEQ ID NO: 1), B. licheniformis YuaB (SEQ ID NO: 2), B. amyloliquefaciens YuaB (SEQ ID NO: 3), B. pumilus YuaB (SEQ ID NO: 4), and B. subtilis YweA (SEQ ID NO: 5) have been used in multiphasic systems (see WO2016027078). Based on phylogenetic analysis (see FIGS. 1, 2, 3 and 4), these BslA proteins can be classified as Class I BslA proteins (or YuaB-like) (SEQ ID NO: 1, SEQ ID NO: 2, SEQ ID NO: 3, and SEQ ID NO: 4) and Class II BslA proteins (or YweA-like) (SEQ ID NO: 5).

As part of the current invention, genome mining of NCBI protein databases using NCBI BLASTp searched with default parameters using the Advanced Search (found at http://www.ncbi.nih.gov/blast/) allowed the identification of several BslA proteins encoded by bacteria from different genera (such as Thermoactinomyces (SEQ ID NO: 6), Jeotgalibacillus, Streptococcus, and Micobacterium), demonstrating that BslA proteins with predicted hydrophobic caps are not exclusively produced by the genus Bacillus.

Furthermore, some of the identified proteins have low homology and less than 50% amino acid identity compared to the Class I BslA (YuaB-like) and Class II BslA (YweA-like) proteins and belong to two different phylogenetic groups, i.e. Class III BslA proteins with sequence similarity to Thermoactinomyces vulgaris BslA (SEQ ID NO: 6) and Class IV BslA proteins that include proteins from B. licheniformis (SEQ ID NOs: 7, 8, 9, 10, and 11), B. glycinifermentans (SEQ ID NOs: 12, 13, 14, and 15), B. sonorensis (SEQ ID NO: 16), B. paralicheniformis (SEQ ID NOs: 17, and 18), Bacillus sp. (SEQ ID NOs: 19, 20, 21, and 22), and B. amyloliquefaciens.

To our knowledge, only one member of Class III BslA proteins (SEQ ID NO: 6) has been deposited in protein sequence databases. This protein has an amino acid identity lower than 30% when compared to the Classes I, II, and IV BslA proteins described above.

In contrast, several examples of Class IV BslA proteins were identified by genome mining. These Class IV BslAs proteins (SEQ ID NOs: 7 to 22) have high homology at the C-terminus and a consensus sequence (SED ID NO: 23):

SNKEWXTSDIEXTYXPNXFVGXSXVEFXFPYRFHAXTRDXLNGXXLXY
TQILNDGQTVRVPVYAXSSSXFKLVMXRKTLPNAGTHXXTAELXXXGX
XXXHAEXXXXIXPR

wherein X represents any amino acid.

Unexpectedly, the Applicants found that, Class III and Class IV BslA proteins are able to produce significant sudsing in personal care compositions. Not wishing to be bound by theory, the Applicants believe that the sudsing benefits are due to the amino acid sequences and/or protein structures thereby enhancing the adsorption at the interface between two phases (oil/water or air/water).

In one embodiment of the present invention, a personal care composition of the present invention comprises one or more BslA proteins, wherein said BslA proteins are a Class III or a Class IV BslA protein. The Class III BslA protein has at least 50%, preferably at least 60%, preferably at least 70%, preferably at least 80%, preferably at least 85%, preferably at least 90%, preferably at least 95%, preferably at least 98% or even 100% amino acid identity to at least one wild-type protein selected from the group consisting of: Thermoactinomyces vulgaris BslA (SEQ ID NO: 6). The Class IV BslA protein has at least 50%, preferably at least 60%, preferably at least 70%, preferably at least 80%, preferably at least 85%, preferably at least 90%, preferably at least 95%, preferably at least 98% or even 100% amino acid identity to at least one wild-type protein selected from the group consisting of: Bacillus licheniformis BslA (SEQ ID NO: 7).

Preferably the personal care composition of the present invention comprises one or more BslA proteins, wherein said BslA proteins are a Class III or a Class IV BslA protein, wherein the Class III BslA protein has at least 90%, preferably at least 95%, preferably at least 98% or even 100% amino acid identity to a wild-type protein: Thermoactinomyces vulgaris BslA (SEQ ID NO: 6), and wherein the Class IV BslA protein has at least 90%, preferably at least 95%, preferably at least 98% or even 100% amino acid identity to at least one wild-type protein sequence selected from the group consisting of: B. licheniformis BslA (SEQ ID NO: 7, SEQ ID NO: 8, SEQ ID NO: 9, SEQ ID NO: 10, and SEQ ID NO: 11), B. glycinifermentans BslA (SEQ ID NO: 12, SEQ ID NO: 13, SEQ ID NO: 14, and SEQ ID NO: 15), B. sonorensis BslA (SEQ ID NO: 16), B. paralicheniformis BslA (SEQ ID NO: 17, and SEQ ID NO: 18), and Bacillus sp. BslA (SEQ ID NO: 19, SEQ ID NO: 20, SEQ ID NO: 21, and SEQ ID NO: 22), preferably wherein the Class IV BslA protein preferably having at least 90%, preferably at least 95%, preferably at least 98% or even 100% amino acid identity to B. licheniformis BslA (SEQ ID NO: 7).

Preferably the personal care composition of the present invention comprises one or more BslA proteins, wherein said BslA proteins are a Class IV BslA protein having at least 90%, preferably at least 95%, preferably at least 98% or even 100% amino acid identity to a protein having the amino acid sequence SED ID NO: 23.

The invention also includes variants in the form of truncated forms derived from a wild type BslA protein, such as a protein with a truncated N-terminus. Most of Class III or Class IV BslA proteins are predicted to include an N-terminal signal peptide that is likely removed upon secretion by the native organisms. The current invention may also include variants without the N-terminal signal peptide. For example, SEQ ID NO: 24, which corresponds to the sequence of full length wild-type Thermoactinomyces vulgaris BslA (SEQ ID NO: 6) without the predicted N-terminal signal peptide, is also part of the current invention. Bioinformatic tools, such as for example, signal peptide prediction server SignalP version 4.1 (Petersen T N., Brunak S., von Heijne G. and Nielsen H. (2011). Nature Methods, 8:785-786), can be used to predict the existence and length of such signal peptides. The invention also includes variants derived by adding an extra amino acid sequence to a wild-type protein, such as for example, an N-terminal tag, a C-terminal tag or an insertion in the middle of the protein sequence. Non-limiting examples of tags are maltose binding protein (MBP) tag, glutathione S-transferase (GST) tag, thioredoxin (Trx) tag, His-tag, and any other tags known by those skilled in art. Tags can be used to improve solubility and expression levels during fermentation or as a handle for enzyme purification.

Preferably the personal care composition of the present invention comprises one or more BslA proteins, wherein said BslA proteins is a Class IV BslA protein having at least 90%, preferably at least 95%, preferably at least 98% or even 100% amino acid identity to a protein having the amino acid sequence SED ID NO: 24.

It is important that variants of Class III or Class IV BslA proteins retain or even improve the ability of the wild-type proteins to adsorb at an interface and to stabilize that interface. Some performance drop in a given property of Class III or Class IV BslA protein variants may of course be tolerated, but the Class III or Class IV BslA protein variants should retain suitable properties for the relevant application for which they are intended. For instance, screening of variants of one of the wild-types can be used to identify whether they retain appropriate properties.

Suitable examples of Class III or Class IV BslA protein variants include one conservative substitution in the peptide, such as a conservative substitution in SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, or SEQ ID NO: 22.

Other suitable examples of Class III or Class IV BslA protein variants include 10 or fewer conservative substitutions in the peptide, such as five or fewer. The Class III or Class IV BslA proteins of the invention may therefore include 1, 2, 3, 4, 5, 6, 7, 8, 9, 10 or more conservative substitutions. The Class III or Class IV BslA proteins can be produced to contain one or more conservative substitutions by manipulating the nucleotide sequence that encodes them using, for example, standard procedures such as site-directed mutagenesis or PCR. Alternatively, the Class III or Class IV BslA proteins can be produced to contain one or more conservative substitutions by using peptide synthesis methods, for example, as known in the art.

Examples of amino acids which may be substituted for an original amino acid in a Class III or Class IV BslA protein and which are regarded as conservative substitutions include: Ser for Ala; Lys for Arg; Gln or His for Asn; Glu for Asp; Asn for Gln; Asp for Glu; Pro for Gly; Asn or Gln for His; Leu or Val for Ile; Be or Val for Leu; Arg or Gln for Lys; Leu or Ile for Met; Met, Leu or Tyr for Phe; Thr for Ser; Ser for Thr; Tyr for Trp; Trp or Phe for Tyr; and Ile or Leu for Val.

The Class III or Class IV BslA proteins of the invention may comprise variants of SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, or SEQ ID NO: 22, wherein a short amino acid sequence containing two cysteine residues is added at the C-terminus. These cysteine residues can allow the Class III or Class IV BslA protein variants to form multimers (i.e., dimers, tetramers, hexamers and potentially higher order oligomers) in solution due to the formation of disulfide bonds between the cysteine residues of adjacent Class III or Class IV BslA protein variants.

The Class III or Class IV BslA proteins of the invention may also cover any fragments of SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, or SEQ ID NO: 22. Preferably the Class III or Class IV BslA protein fragments can adsorb to an interface and stabilize that interface.

The Class III or Class IV BslA proteins can be modified by a variety of chemical techniques to produce derivatives having essentially the same or even improved activity as the unmodified peptides, and optionally having other desirable properties. For example, carboxylic acid groups of the protein, whether carboxyl-terminal or side chain, may be provided in the form of a salt of a pharmaceutically-acceptable cation or esterified, for example to form a C1-C6 alkyl ester, or converted to an amide, for example of formula CONR₁R₂ wherein R₁ and R₂ are each independently H or C1-C6 alkyl, or combined to form a heterocyclic ring, such as a 5- or 6-membered ring. Amino groups of the peptide, whether amino-terminal or side chain, may be in the form of a pharmaceutically-acceptable acid addition salt, such as the HCI, HBr, acetic, benzoic, toluene sulfonic, maleic, tartaric and other organic salts, or may be modified to C1-C6 alkyl or dialkyl amino or further converted to an amide. Hydroxyl groups of the peptide side chains may be converted to alkoxy or ester groups, for example C1-C6 alkoxy or C1-C6 alkyl ester, using well-recognized techniques. Phenyl and phenolic rings of the peptide side chains may be substituted with one or more halogen atoms, such as F, Cl, Br or I, or with C1-C6 alkyl, C1-C6 alkoxy, carboxylic acids and esters thereof, or amides of such carboxylic acids. Methylene groups of the peptide side chains can be extended to homologous C2-C4 alkylenes. Thiols can be protected with any one of a number of well-recognized protecting groups, such as acetamide groups. Those skilled in the art will also recognize methods for introducing cyclic structures into the BslA proteins of the present invention to select and provide conformational constraints to the structure that result in enhanced stability.

Identity, or homology, percentages as mentioned herein in respect of the present invention are those that can be calculated with the GAP program, obtainable from GCG (Genetics Computer Group Inc., Madison, Wis., USA). Alternatively, a manual alignment can be performed.

For polypeptide sequence comparison the following settings can be used: Alignment algorithm: Needleman and Wunsch, J. Mol. Biol. 1970, 48: 443-453. As a comparison matrix for amino acid similarity the Blosum62 matrix is used (Henikoff S. and Henikoff J. G., P.N.A.S. USA 1992, 89: 10915-10919). The following gap scoring parameters are used: Gap penalty: 12, gap length penalty: 2, no penalty for end gaps.

A given sequence is typically compared against the full-length sequence of SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, or SEQ ID NO: 22 to obtain a score. Preferably, the BslA proteins are present in an amount from 0.01 wt % to 5 wt %, preferably from 0.1 wt % to 1 wt %, by weight of said personal care composition based on active protein, wherein the BslA proteins are selected from Class III or Class IV BslA proteins. The Class III BslA protein has at least 50%, preferably at least 60%, preferably at least 70%, preferably at least 80%, preferably at least 85%, preferably at least 90%, preferably at least 95%, preferably at least 98% or even 100% amino acid identity to a wild-type protein: Thermoactinomyces vulgaris BslA (SEQ ID NO: 6), and the Class IV BslA protein has at least 50%, preferably at least 60%, preferably at least 70%, preferably at least 80%, preferably at least 85%, preferably at least 90%, preferably at least 95%, preferably at least 98% or even 100% amino acid identity to a wild-type protein: Bacillus licheniformis BslA (SEQ ID NO: 7). More preferably the Class III BslA protein has at least 90%, preferably at least 95%, preferably at least 98% or even 100% amino acid identity to a wild-type protein: Thermoactinomyces vulgaris BslA (SEQ ID NO: 6), and the Class IV BslA protein has at least 90%, preferably at least 95%, preferably at least 98% or even 100% amino acid identity to at least one wild-type protein sequence selected from the group consisting of: B. licheniformis BslA (SEQ ID NO: 7, SEQ ID NO: 8, SEQ ID NO: 9, SEQ ID NO: 10, and SEQ ID NO: 11), B. glycinifermentans BslA (SEQ ID NO: 12, SEQ ID NO: 13, SEQ ID NO: 14, and SEQ ID NO: 15), B. sonorensis BslA (SEQ ID NO: 16), B. paralicheniformis BslA (SEQ ID NO: 17, and SEQ ID NO: 18), and Bacillus sp. BslA (SEQ ID NO: 19, SEQ ID NO: 20, SEQ ID NO: 21, and SEQ ID NO: 22), the Class IV BslA protein most preferably having at least 90%, preferably at least 95%, preferably at least 98% or even 100% amino acid identity to B. licheniformis BslA (SEQ ID NO: 7).

### III. Chaplin Proteins

The personal care composition in accordance with the present invention can comprise one or more chaplin proteins. Chaplin proteins (coelicolor hydrophobic aerial proteins) were originally discovered in Streptomyces coelicolor, but genome mining in protein databases indicate that these proteins are broadly distributed, including species in Actinobacteria, Cyanobacteria, Firmicutes, and even Fungi.

Chaplin proteins share significant sequence identity, including a highly conserved chaplin domain of approximately 40 amino acids, usually referred as DUF320 (pfam03777). The consensus sequence of the DUF320 domain is shown in SEQ ID NO: 33. A “chaplin protein” of the present invention is any protein containing at least one DUF320 domain and with a length of less than about 350 amino acids

Proteins containing multiple DUF320 domains have been deposited on protein sequence databases. S. coelicolor A32 produces eight different chaplin proteins (ChpA-H). For example, S. coelicolor ChpA (SEQ ID NO: 34), S. coelicolor ChpB (SEQ ID NO: 35) and S. coelicolor ChpC (SEQ ID NO: 36) contain two N-terminal DUF320 domains and a C-terminal cell wall anchoring domain, whereas S. coelicolor ChpD (SEQ ID NO: 37), S. coelicolor ChpE (SEQ ID NO: 38), S. coelicolor ChpF (SEQ ID NO: 39), S. coelicolor ChpG (SEQ ID NO: 40) and S. coelicolor ChpH (SEQ ID NO: 41) are shorter and contain an N-terminal secretion signal peptide and a C-terminal DUF320 domain. Other species of Streptomyces also produce chaplin proteins. For example, a predicted chaplin from S. pristinaespiralis (SEQ ID NO: 45) contain an N-terminal signal peptide, a DUF320 domain, and an extra few amino acids at the C-terminus with unknown function.

Other bacterial species, e.g. Catenulispora acidiphila, are predicted to produce several chaplin proteins with different domain architectures. Similarly to ChpD-H from S. coelicolor, two predicted C. acidiphila chaplin proteins (SEQ ID NO: 42 and SEQ ID NO: 43) are short and contain only an N-terminal secretion signal peptide and a C-terminal DUF320 domain. Another C. acidiphila chaplin (SEQ ID NO: 46) contains an N-terminal secretion signal peptide, four DUF320 domains, and a C-terminal cell wall anchoring domain. Chaplin proteins with different domain architecture are part of the current invention.

Even though several amino acids are highly conserved in different chaplin proteins, the sequence identity of chaplin proteins can be pretty low. For example, the predicted chaplin from Conidiobolus coronatus (SEQ ID NO: 53) has between 18% and 21% sequence identity when compared to ChpD-H.

Frequently, chaplin proteins contain two cysteine residues (e.g., ChpD, ChpF, ChpG, and ChpH) that may be involved in disulfide bond formation, perhaps enabling heteropolymerization and creating longer structures. In other examples, the cysteine residues are not present (e.g., ChpE). Chaplin proteins with or without cysteine residues are part of the current invention. Furthermore, a diverse number of proteins contain the DUF320 domain in combination with other domains, which may add different functions. These proteins are also part of the current invention.

The role of chaplin proteins in S. coelicolor is to coat the aerial hyphae assisting spore dispersal and colonization of surrounding soil, while different chaplin proteins can adopt distinct roles in vivo. For example, S. coelicolor ChpE and ChpH are expressed at high levels in the vegetative and aerial mycelial phases and likely perform two different functions: lowering the surface tension of water (i.e., as surfactants) and assembling into a hydrophobic layer to coat the emerging hyphae. In contrast, the other chaplin proteins are only expressed during the aerial hyphae formation and may only contribute to the later role.

Unexpectedly, the Applicants found that chaplin proteins are able to generate sudsing in personal care cleaning formulations comprising a surfactant. Not wishing to be bound by theory, the Applicants believe that the increased sudsing benefits are due to the specific amino acid sequences and/or protein structures enhancing the adsorption at the interface between two phases (oil/water or air/water).

Preferably the chaplin proteins have at least 50%, preferably at least 60%, preferably at least 70%, preferably at least 80%, preferably at least 85%, preferably at least 90%, preferably at least 95%, preferably at least 98% or even 100% amino acid identity to at least one protein selected from the group consisting of SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, or SEQ ID NO: 55, more preferably to at least one wild-type protein selected from the group consisting of: Streptomyces coelicolor ChpD (SEQ ID NO: 48), Streptomyces coelicolor ChpE (SEQ ID NO: 49), Streptomyces coelicolor ChpF (SEQ ID NO: 50), Streptomyces coelicolor ChpG (SEQ ID NO: 51), and Streptomyces coelicolor ChpH (SEQ ID NO: 52), more preferably Streptomyces coelicolor ChpE (SEQ ID NO: 49), and Streptomyces coelicolor ChpF (SEQ ID NO: 50).

Preferably the chaplin proteins have at least 50%, preferably at least 60%, preferably at least 70%, preferably at least 80%, preferably at least 85%, preferably at least 90%, preferably at least 95%, preferably at least 98% or even 100% amino acid identity to at least one wild-type protein selected from the group consisting of: SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, and SEQ ID NO: 55.

Preferably the chaplin proteins have at least 50%, preferably at least 60%, preferably at least 70%, preferably at least 80%, preferably at least 85%, preferably at least 90%, preferably at least 95%, preferably at least 98% or even 100% amino acid identity to the DUF320 consensus sequence SEQ ID NO: 1. Preferably the chaplin proteins comprise at least one DUF320 domain.

The invention also includes chaplin protein variants. For example, chaplin protein variants, as used herein, include a sequence resulting when a wild-type protein is modified by, or at, one or more amino acids (for example 1, 2, 5 or 10 amino acids). The invention also includes chaplin protein variants in the form of truncated forms derived from a wild-type chaplin, such as a wild-type chaplin protein with a truncated N-terminus or a truncated C-terminus.

Majority of chaplin proteins are predicted to include an N-terminal signal peptide that is likely removed upon secretion by the native organisms. Preferably the chaplin protein variants of the present invention are without the N-terminal signal peptide. For example, SEQ ID NO: 48, SEQ ID NO: 49, SEQ ID NO: 50, SEQ ID NO: 51, and SEQ ID NO: 52 are variants of the full length wild-type Streptomyces coelicolor ChpD-H (SEQ ID NO: 37, SEQ ID NO: 38, SEQ ID NO: 39, SEQ ID NO: 40, and SEQ ID NO: 41, respectively) without the N-terminal signal peptide. Bioinformatic tools, such as SignalP version 4.1 (Petersen T N., Brunak S., von Heijne G. and Nielsen H. (2011), Nature Methods, 8:785-786), can be used to predict the existence and length of such signal peptides.

Some chaplin proteins may contain a C-terminal cell wall anchoring domain or a transmembrane domain. Preferably the present invention includes chaplin protein variants without such domains. Bioinformatic tools, such as TMHMM by the Center for Biological Sequence Analysis at the Technical University of Denmark, can be used to predict the existence and length of such domains.

The invention also includes variants derived by adding an extra amino acid sequence to a wild-type protein, such as for example, an N-terminal tag, a C-terminal tag or an insertion in the middle of the protein sequence. Non-limiting examples of tags are maltose binding protein (MBP) tag, glutathione S-transferase (GST) tag, thioredoxin (Trx) tag, His-tag, and any other tags known by those skilled in art. Tags can be used to improve solubility and expression levels during fermentation or as a handle for enzyme purification. For example, His6-MBP-TEV_ChpF (SEQ ID NO: 55) is a variant of ChpF (SEQ ID NO: 39) including N-terminal His and MBP tags.

It is important that variants of chaplin proteins retain or preferably improve the ability of the wild-type proteins to adsorb at an interface and to stabilize that interface. Some performance drop in a given property of chaplin protein variants may of course be tolerated, but the chaplin protein variants should retain or preferably improve suitable properties for the relevant application for which they are intended. For instance, screening of variants of one of the wild-types can be used to identify whether they retain or improve appropriate properties.

Suitable examples of chaplin protein variants include one conservative substitution in the peptide, such as a conservative substitution in SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, or SEQ ID NO: 55.

Other suitable examples of chaplin protein variants include 10 or fewer conservative substitutions are included in the peptide, such as five or fewer. The chaplin proteins of the present invention may therefore include 1, 2, 3, 4, 5, 6, 7, 8, 9, 10 or more conservative substitutions. The chaplin proteins can be produced to contain one or more conservative substitutions by manipulating the nucleotide sequence that encodes that peptide using, for example, standard procedures such as site-directed mutagenesis or PCR. Alternatively, the chaplin proteins can be produced to contain one or more conservative substitutions by using peptide synthesis methods, for example, as known in the art.

Examples of amino acids which may be substituted for an original amino acid in a chaplin protein and which are regarded as conservative substitutions include: Ser for Ala; Lys for Arg; Gln or His for Asn; Glu for Asp; Asn for Gln; Asp for Glu; Pro for Gly; Asn or Gln for His; Leu or Val for Ile; Ile or Val for Leu; Arg or Gln for Lys; Leu or Ile for Met; Met, Leu or Tyr for Phe; Thr for Ser; Ser for Thr; Tyr for Trp; Trp or Phe for Tyr; and Ile or Leu for Val.

Preferably the chaplin proteins of the invention may comprise variants of SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, or SEQ ID NO: 55 wherein one or more cysteine residues are substituted by another amino acid.

Preferably the chaplin proteins of the present invention may comprise variants of SEQ ID NO: 6 or SEQ ID NO: 17, wherein a short amino acid sequence containing two cysteine residues is added at the C-terminus or at least two residues are modified to cysteines. These cysteine residues can allow the chaplin proteins to form multimers (i.e., dimers, tetramers, hexamers and potentially higher order oligomers) in solution due to the formation of disulfide bonds between the cysteine residues of adjacent chaplin protein variants.

The chaplin proteins of the present invention may also cover fragments of SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, or SEQ ID NO: 55. Preferably the chaplin protein fragments can adsorb to an interface and stabilize that interface.

The chaplin proteins can be modified by a variety of chemical techniques to produce derivatives having essentially the same or even improved activity as the unmodified peptides, and optionally having other desirable properties. For example, carboxylic acid groups of the protein, whether carboxyl-terminal or side chain, may be provided in the form of a salt of a pharmaceutically-acceptable cation or esterified, for example to form a C1-C6 alkyl ester, or converted to an amide, for example of formula CONR₁R₂ wherein R₁ and R₂ are each independently H or C1-C6 alkyl, or combined to form a heterocyclic ring, such as a 5- or 6-membered ring. Amino groups of the peptide, whether amino-terminal or side chain, may be in the form of a pharmaceutically-acceptable acid addition salt, such as the HCI, HBr, acetic, benzoic, toluene sulfonic, maleic, tartaric and other organic salts, or may be modified to C1-C6 alkyl or dialkyl amino or further converted to an amide. Hydroxyl groups of the peptide side chains may be converted to alkoxy or ester groups, for example C1-C6 alkoxy or C1-C6 alkyl ester, using well-recognized techniques. Phenyl and phenolic rings of the peptide side chains may be substituted with one or more halogen atoms, such as F, Cl, Br or I, or with C1-C6 alkyl, C1-C6 alkoxy, carboxylic acids and esters thereof, or amides of such carboxylic acids. Methylene groups of the peptide side chains can be extended to homologous C2-C4 alkylenes. Thiols can be protected with any one of a number of well-recognized protecting groups, such as acetamide groups. Those skilled in the art will also recognize methods for introducing cyclic structures into the chaplin proteins of the present invention to select and provide conformational constraints to the structure that result in enhanced stability.

Identity, or homology, percentages as mentioned herein in respect of the present invention are those that can be calculated with the GAP program, obtainable from GCG (Genetics Computer Group Inc., Madison, Wis., USA). Alternatively, a manual alignment can be performed.

For polypeptide sequence comparison the following settings can be used: Alignment algorithm: Needleman and Wunsch, J. Mol. Biol. 1970, 48: 443-453. As a comparison matrix for amino acid similarity the Blosum62 matrix is used (Henikoff S. and Henikoff J. G., P.N.A.S. USA 1992, 89: 10915-10919). The following gap scoring parameters are used: Gap penalty: 12, gap length penalty: 2, no penalty for end gaps.

A given sequence is typically compared against the full-length sequence of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, or SEQ ID NO: 55 to obtain a score.

The personal care composition preferably comprises from 0.001 wt % to 5 wt %, preferably from 0.1 wt % to 1 wt %, by weight of said composition based on active protein of one or more chaplin proteins. Preferably said chaplin protein has at least 50%, preferably at least 60%, preferably at least 70%, preferably at least 80%, preferably at least 85%, preferably at least 90%, preferably at least 95%, preferably at least 98% or even 100% amino acid identity to at least one protein selected from the group consisting of SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, or SEQ ID NO: 55. More preferably said chaplin protein has at least 50%, preferably at least 60%, preferably at least 70%, preferably at least 80%, preferably at least 85%, preferably at least 90%, preferably at least 95%, preferably at least 98% or even 100% amino acid identity to at least one wild-type protein selected from the group consisting of: Streptomyces coelicolor ChpD (SEQ ID NO: 48), Streptomyces coelicolor ChpE (SEQ ID NO: 49), Streptomyces coelicolor ChpF (SEQ ID NO: 50), Streptomyces coelicolor ChpG (SEQ ID NO: 51), and Streptomyces coelicolor ChpH (SEQ ID NO: 52). Most preferably said chaplin protein has at least 50%, preferably at least 60%, preferably at least 70%, preferably at least 80%, preferably at least 85%, preferably at least 90%, preferably at least 95%, preferably at least 98% or even 100% amino acid identity to at least one wild-type protein selected from the group consisting of: Streptomyces coelicolor ChpE (SEQ ID NO: 49) and Streptomyces coelicolor ChpF (SEQ ID NO: 50).

### IV. Ranaspumins and Latherins

Ranaspumins (from Latin: rana (frog) and spuma (foam)) are proteins originally characterized from the suds nest material produced by the tungara frog (Engystomops pustulosus). In this particular specie, six main ranaspumins (designated as Ep-Rsn1, Ep-Rsn2, Ep-Rsn3, Ep-Rsn4, Ep-Rsn5, and Ep-Rsn6) with different biological roles related to suds formation and stability have been identified. From these proteins, Ep-Rsn2 (SEQ ID NO: 56) is the major surface active protein in the suds mixture, while the other ranaspumins contribute mostly to suds stability. Ep-Rsn2 has no homology to any other protein or domains presently reported in the protein sequences databases. Interestingly, the Ep-Rsn2 sequence shows an unusual distribution of amino acid residues, including a highly hydrophobic N-terminal region (LILDGDLLK-) and a highly charged C-terminal region (-RKDDDDDDGY), suggesting its potential role as a surface activity protein. Structural analysis revealed that Ep-Rsn2 comprises a four-stranded antiparallel β sheet with an a helix lying across one side of the sheet, similar to cystatins. The flexible N-terminal unstructured tail is expected to capture hydrophobic interfaces, followed by a large conformational change where the helix moves apart from the sheet revealing the hydrophobic core of the protein.

Protein Ep-Rsn1 (SEQ ID NO: 60) has some amino acid sequence similarity to cystatins (cysteinyl proteinase inhibitors), but does not appear to have similar inhibitory activity. Instead, Ep-Rsn1 reduces aqueous surface tension, though not at the same level than Ep-Rsn1. Proteins Ep-Rsn3 (SEQ ID NO: 61), Ep-Rsn4 (SEQ ID NO: 62), and Ep-Rsn5 (SEQ ID NO: 63) are similar to each other and have some sequence similarity to a family of fucose-binding proteins, also known as “fucolectins”, whereas Ep-Rsn6 (SEQ ID NO: 64) belongs to a different type of lectins (C-type) frequently associated with galactose binding. The carbohydrate-binding activity of Ep-Rsn4 has been confirmed experimentally. Furthermore, Ep-Rsn3 and Ep-Rsn5 have hydrophobic N-terminal tails that might serve to anchor them at the interface.

The role of Ep-Rsn3, Ep-Rsn4, Ep-Rsn5, and Ep-Rsn6 in suds stabilization has been suggested in the art. It is believed that initial suds formation is facilitated by Ep-Rsn2 (and possibly Ep-Rsn1), while the rest of the ranaspumins build a more complex layer, possibly by binding to long-chain branched polysaccharide molecules, creating a mechanically stable interface. Indeed, the suds from E. pustulosus contain not only proteins, but also significant amounts of carbohydrates, predominantly complex cross-linked mixtures of O- and N-glycans.

Composition analysis of the suds nests of Leptodactylus vastus, an unrelated frog species, allowed the identification of a mixture of proteins, including the surface active protein Lv-Rsn1 (SEQ ID NO: 57). This protein is much bigger than Ep-Rsn2 and comprises two domains and four disulfide bridges that stabilize the structure. It is believed that Lv-Rsn1 undergoes a conformational change to facilitate interfacial association. Despite similar functions, Lv-Rsn1 is totally unrelated to Ep-Rsn2, but has homology to proteins produced by Leptodactylus fuscus, designed as Lf-Rsn1 (SEQ ID NO: 58), and from Bufo gargarizans, designated as Bg-Rsn1 (SEQ ID NO: 59).

Latherins are proteins found in sweat and saliva of horses and other equines. One of the biological roles of latherins is enabling wetting of the oily, waterproof hairs, aiding fast flow of sweat from the glands, through the thick pelts, to the air interface. The amino acid sequences of latherin from different equine species are highly conserved. They belong to the group of PLUNC (palate, lung, and nasal epithelium clone) proteins expressed in mammalian salivary glands and oral cavities.

The amino acid sequence of Equus caballus latherin (SEQ ID NO: 10) is characterized by an unusually high leucine content (about 24%), which may be related to its surface properties. However, the solution structure of latherin does not display any major hydrophobic regions, suggesting that conformational changes might be required for interfacial association of the protein.

Unexpectedly, the Applicants found that one or more surface active proteins, in particular, surface active proteins selected from the group consisting of ranaspumins, latherins, and mixtures thereof, preferably ranaspumins, is able to produce a more stable hence longer lasting sudsing profile when formulated in personal care compositions. Not wishing to be bound by theory, the Applicants believe that the increased sudsing benefits are due to conformational changes of the proteins that expose hydrophobic patches and generate amphiphilic structures that can associate and stabilize interfaces (i.e., oil-water interface or air-water interface).

Accordingly, the personal care composition in accordance with the present invention comprises one or more surface active proteins selected from the group consisting of ranaspumins, latherins, and mixtures thereof, preferably ranaspumins.

Preferably the ranaspumins have at least 40%, preferably at least 50%, preferably at least 60%, preferably at least 70%, preferably at least 80%, preferably at least 85%, preferably at least 90%, preferably at least 95%, preferably at least 98% or even 100% amino acid identity to at least one wild-type protein selected from the group consisting of: Engystomops pustulosus Ep-Rsn2 (SEQ ID NO: 56), Leptodactylus vastus Lv-Rsn1 (SEQ ID NO: 57), Leptodactylus fuscus Lf-Rsn1 (SEQ ID NO: 58), and Bufo gargarizans Bg-Rsn (SEQ ID NO: 59), more preferably Engystomops pustulosus Ep-Rsn2 (SEQ ID NO: 55) and Leptodactylus vastus Lv-Rsn1 (SEQ ID NO: 56).

Preferably the personal care composition further comprises one or more co-proteins selected from the group of lactins. Non-limiting examples of lactins are Engystomops pustulosus Ep-Rsn3 (SEQ ID NO: 61), Engystomops pustulosus Ep-Rsn4 (SEQ ID NO: 62), Engystomops pustulosus Ep-Rsn5 (SEQ ID NO: 63), and Engystomops pustulosus Ep-Rsn6 (SEQ ID NO: 64).

Preferably the personal care composition further comprises one or more co-proteins wherein the co-proteins have at least 40%, preferably at least 50%, preferably at least 60%, preferably at least 70%, preferably at least 80%, preferably at least 85%, preferably at least 90%, preferably at least 95%, preferably at least 98% or even 100% amino acid identity to at least one wild-type protein selected from the group consisting of: Engystomops pustulosus Ep-Rsn1 (SEQ ID NO: 60), Engystomops pustulosus Ep-Rsn3 (SEQ ID NO: 61), Engystomops pustulosus Ep-Rsn4 (SEQ ID NO: 62), Engystomops pustulosus Ep-Rsn5 (SEQ ID NO: 63), and Engystomops pustulosus Ep-Rsn6 (SEQ ID NO: 64); and mixtures thereof, preferably Engystomops pustulosus Ep-Rsn3 (SEQ ID NO: 61) and Engystomops pustulosus Ep-Rsn5 (SEQ ID NO: 63).

Preferably the latherins have at least 40%, preferably at least 50%, preferably at least 60%, preferably at least 70%, preferably at least 80%, preferably at least 85%, preferably at least 90%, preferably at least 95%, preferably at least 98% or even 100% amino acid identity to Equus caballus latherin (SEQ ID NO: 65).

The present invention also includes variants of ranaspumins and latherins. Variants of ranaspumins or latherins, as used herein, include a sequence resulting when a wild-type protein of the respective protein is modified by, or at, one or more amino acids (for example 1, 2, 5 or 10 amino acids). The invention also includes variants in the form of truncated forms derived from a wild-type ranaspumin or wild type latherin, such as a protein with a truncated N-terminus or a truncated C-terminus. Some ranaspumins (e.g., Ep-Rsn1, Ep-Rsn4, and Ep-Rsn5) and latherin (SEQ ID NO: 10) are predicted to include an N-terminal signal peptide that is likely removed upon secretion by the cell. The present invention includes variants without the N-terminal signal peptide. Bioinformatic tools, such as SignalP ver 4.1 (Petersen T N., Brunak S., von Heijne G. and Nielsen H. (2011), Nature Methods, 8:785-786), can be used to predict the existence and length of such signal peptides. The invention also includes variants derived by adding an extra amino acid sequence to a wild-type protein, such as for example, an N-terminal tag, a C-terminal tag or an insertion in the middle of the protein sequence. Non-limiting examples of tags are maltose binding protein (MBP) tag, glutathione S-transferase (GST) tag, thioredoxin (Trx) tag, His-tag, and any other tags known by those skilled in art. Tags can be used to improve solubility and expression levels during fermentation or as a handle for enzyme purification. For example, His6-Ep-Rns2 (SEQ ID NO: 67) is a variant of Ep-Rns2 (SEQ ID NO: 56) including an N-terminal His tag and His6-Lv-Rns1 (SEQ ID NO: 69) is a variant of Lv-Rns1 (SEQ ID NO: 57) also including the same tag.

It is important that variants of ranaspumins and latherins retain and preferably improve the ability of the wild-type protein to adsorb at an interface and to stabilize that interface. Some performance drop in a given property of variants may of course be tolerated, but the variants should retain and preferably improve suitable properties for the relevant application for which they are intended. Screening of variants of one of the wild-types can be used to identify whether they retain and preferably improve appropriate properties.

The variants may have “conservative” substitutions. Suitable examples of conservative substitution includes one conservative substitution in the peptide, such as a conservative substitution in SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 67, or SEQ ID NO: 69. Other suitable examples include 10 or fewer conservative substitutions in the peptide, such as five or fewer. A peptide or protein of the invention may therefore include 1, 2, 3, 4, 5, 6, 7, 8, 9, 10 or more conservative substitutions. A peptide can be produced to contain one or more conservative substitutions by manipulating the nucleotide sequence that encodes that peptide using, for example, standard procedures such as site-directed mutagenesis or PCR. Alternatively, a peptide can be produced to contain one or more conservative substitutions by using peptide synthesis methods, for example, as known in the art.

Examples of amino acids which may be substituted for an original amino acid in a protein and which are regarded as conservative substitutions include: Ser for Ala; Lys for Arg; Gln or His for Asn; Glu for Asp; Asn for Gln; Asp for Glu; Pro for Gly; Asn or Gln for His; Leu or Val for Ile; Ile or Val for Leu; Arg or Gln for Lys; Leu or Ile for Met; Met, Leu or Tyr for Phe; Thr for Ser; Ser for Thr; Tyr for Trp; Trp or Phe for Tyr; and Ile or Leu for Val.

A variant includes a “modified protein” which encompasses proteins having at least one substitution, insertion, and/or deletion of an amino acid. A modified protein may have 1, 2, 3, 4, 5, 6, 7, 8, 9 or 10 or more amino acid modifications (selected from substitutions, insertions, deletions and combinations thereof).

The invention also covers any fragment of SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 67, or SEQ ID NO: 69 that can adsorb to an interface and stabilize that interface. According to the invention, the term “fragment” is intended to mean an amino acid sequence of at least 20, 40, 60, 80 contiguous amino acids of the reference sequences or any integer there between.

Peptides can be modified by a variety of chemical techniques to produce derivatives having essentially the same or preferably even improved activity as the unmodified peptides, and optionally having other desirable properties. For example, carboxylic acid groups of the protein, whether carboxyl-terminal or side chain, may be provided in the form of a salt of a pharmaceutically-acceptable cation or esterified, for example to form a C1-C6 alkyl ester, or converted to an amide, for example of formula CONR₁R₂ wherein R₁ and R₂ are each independently H or C1-C6 alkyl, or combined to form a heterocyclic ring, such as a 5- or 6-membered ring. Amino groups of the peptide, whether amino-terminal or side chain, may be in the form of a pharmaceutically-acceptable acid addition salt, such as the HCI, HBr, acetic, benzoic, toluene sulfonic, maleic, tartaric and other organic salts, or may be modified to C1-C6 alkyl or dialkyl amino or further converted to an amide. Hydroxyl groups of the peptide side chains may be converted to alkoxy or ester groups, for example C1-C6 alkoxy or C1-C6 alkyl ester, using well-recognized techniques. Phenyl and phenolic rings of the peptide side chains may be substituted with one or more halogen atoms, such as F, Cl, Br or I, or with C1-C6 alkyl, C1-C6 alkoxy, carboxylic acids and esters thereof, or amides of such carboxylic acids. Methylene groups of the peptide side chains can be extended to homologous C2-C4 alkylenes. Thiols can be protected with any one of a number of well-recognized protecting groups, such as acetamide groups. Those skilled in the art will also recognize methods for introducing cyclic structures into the peptides of this disclosure to select and provide conformational constraints to the structure that result in enhanced stability.

Identity, or homology, percentages as mentioned herein in respect of the present invention are those that can be calculated with the GAP program, obtainable from GCG (Genetics Computer Group Inc., Madison, Wis., USA). Alternatively, a manual alignment can be performed.

For polypeptide sequence comparison the following settings can be used: Alignment algorithm: Needleman and Wunsch, J. Mol. Biol. 1970, 48: 443-453. As a comparison matrix for amino acid similarity the Blosum62 matrix is used (Henikoff S. and Henikoff J. G., P.N.A.S. USA 1992, 89: 10915-10919). The following gap scoring parameters are used: Gap penalty: 12, gap length penalty: 2, no penalty for end gaps.

A given sequence is typically compared against the full-length sequence of SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 67, or SEQ ID NO: 69 to obtain a score.

Preferably, the surface active proteins are present in an amount from 0.0001 wt % to 5 wt %, preferably from 0.01 wt % to 1 wt %, by weight of the personal care composition based on active protein, wherein the surface active protein is selected from selected from the group consisting of ranaspumins, latherins, and mixtures thereof, preferably ranaspumins. Preferably the ranaspumins have at least 40%, preferably at least 50%, preferably at least 60%, preferably at least 70%, preferably at least 80%, preferably at least 85%, preferably at least 90%, preferably at least 95%, preferably at least 98% or even 100% amino acid identity to at least one wild-type protein selected from the group consisting of: Engystomops pustulosus Ep-Rsn2 (SEQ ID NO: 56), Leptodactylus vastus Lv-Rsn1 (SEQ ID NO: 57), Leptodactylus fuscus Lf-Rsn1 (SEQ ID NO: 58), and Bufo gargarizans Bg-Rsn (SEQ ID NO: 59), more preferably Engystomops pustulosus Ep-Rsn2 (SEQ ID NO: 56 and Leptodactylus vastus Lv-Rsn1 (SEQ ID NO: 57). Preferably the latherins have at least 40%, preferably at least 50%, preferably at least 60%, preferably at least 70%, preferably at least 80%, preferably at least 85%, preferably at least 90%, preferably at least 95%, preferably at least 98% or even 100% amino acid identity to Equus caballus latherin (SEQ ID NO: 65).

### V. Hydrophobins

As described in Wosten, Annu. Rev. Microbiol. 2001, 55, 625646, hydrophobins are proteins of fungal origin that play a broad range of roles in the growth and development of filamentous fungi. For example, they are involved in the formation of aerial structures and in the attachment of hyphae to hydrophobic surfaces. The mechanisms by which hydrophobins perform their function are based on their property of self-assembling at hydrophobic-hydrophilic interfaces into an amphipathic film. Typically, hydrophobins are divided into classes I and II. The assembled amphipathic films of class II hydrophobins are capable of re-dissolving in a range of solvents (particularly although not exclusively an aqueous ethanol) at room temperature. In contrast, the assembled amphipathic films of class I hydrophobins are much less soluble, re-dissolving only in strong acids such as trifluoroacetic acid or formic acid. Detergent compositions containing hydrophobins are known in the art. For example, US 2009/0101167 describes the use of hydrophobins, particularly fusion hydrophobins, for washing textiles and washing compositions containing them. US 2014/0031272 describes a cleaning composition comprising a hydrophobin and a lipolytic enzyme for removing lipid-based stains from surfaces.

Hydrophobins are polypeptides obtained or obtainable from a microorganism. The microorganism may preferably be a bacteria or a fungus, more preferably a fungus. In this specification the term “hydrophobin” is defined as meaning a polypeptide capable of self-assembly at a hydrophilic/hydrophobic interface, and having the general formula:

(Y1)n-B1-(X1)a-B2-(X2)b-B3-(X3)c-B4-(X4)d-B5-(X5)e-B6-(X6)f-B7-(X7)g-B8-(Y2)m

wherein: m and n are independently 0 to 2000; B1, B2, B3, B4, B5, B6, Bland B8 are each independently amino acids selected from Cys, Leu, Ala, Pro, Ser, Thr, Met or Gly, at least 6 of the residues B1 through B8 being Cys; X1, X2, X3, X4, X5, X6, X7; Y1 and Y2 independently represent any amino acid; a is 1 to 50; b is 0 to 5; c is 1 to 100; d is 1 to 100; e is 1 to 50; f is 0 to 5; g is 1 to 100; m is 0 to 100; and n is 0 to 100.

The compositions of the invention may comprise class II hydrophobins. Class I hydrophobins are not included as part of the current invention. It is known in the art that hydrophobins of classes I and II can be distinguished on a number of grounds, both structurally and based on physical parameters including solubility. As described herein, hydrophobins self-assemble at an interface (especially a water/air interface) into amphipathic interfacial films. The assembled amphipathic films of class I hydrophobins are generally re-solubilised only in strong acids (typically those having a pKa of lower than 4, such as formic acid or trifluoroacetic acid), whereas those of class II are soluble in a wider range of solvents.

In one embodiment, the term “class II hydrophobin” means a hydrophobin having the above-described self-assembly property at a water/air interface, the assembled amphipathic films being capable of redissolving to a concentration of at least 0.1% (w/w) in an aqueous ethanol solution (60% v/v) at room temperature. In contrast, in this embodiment, the term “class I hydrophobin” means a hydrophobin having the above-described self-assembly property but which does not have this specified redissolution property.

In another embodiment the term “class II hydrophobin” means a hydrophobin having the above-described self-assembly property at a water/air interface and the assembled amphipathic films being capable of redissolving to a concentration of at least 0.1% (w/w) in an aqueous sodium dodecyl sulphate solution (2% w/w) at room temperature. In contrast, in this embodiment, the term “class I hydrophobin” means a hydrophobin having the above-described self-assembly property but which does not have this specified redissolution property.

Hydrophobins of classes I and II may also be distinguished by the hydrophobicity/hydrophilicity of a number of regions of the hydrophobin protein. The relative hydrophobicity/hydrophilicity of the various regions of the hydrophobin protein can be established by comparing the hydropathy pattern of the hydrophobin using the method set out in Kyte and Doolittle, J. Mol. Biol., 1982, 157, 105-132. According to the teaching of this reference, a computer program can be used to progressively evaluate the hydrophilicity and hydrophobicity of a protein along its amino acid sequence. For this purpose, the method uses a hydropathy scale (based on a number of experimental observations derived from the literature) comparing the hydrophilic and hydrophobic properties of each of the 20 amino acid side-chains. The program uses a moving-segment approach that continuously determines the average hydropathy within a segment of predetermined length as it advances through the sequence. The consecutive scores are plotted from the amino to the carboxy terminus. At the same time, a midpoint line is printed that corresponds to the grand average of the hydropathy of the amino acid compositions found in most of the sequenced proteins. The method is further described for hydrophobins in Wessels, Adv. Microbial Physiol. 1997, 38, 1-45.

The term “class II hydrophobin” means a hydrophobin having the above-described self-assembly property and in which the region between the residues B3 and B4, i.e. the moiety (X3)c, is predominantly hydrophobic. In contrast, the term “class I hydrophobin” means a hydrophobin having the above-described self-assembly property but in which the region between the residues B3 and B4, i.e. the group (X3)c, is predominantly hydrophilic. Alternatively the region between the residues B7 and B8, i.e. the moiety (X7)g, is predominantly hydrophobic for “class II hydrophobin”, while being predominantly hydrophilic for “class I hydrophobin”.

Structurally class II hydrophobins may also be characterised by their sequences. In one embodiment, the class II hydrophobins used in the present invention have the general formula (I):

(Y1)n-B1-(X1)a-B2-B3-(X3)c-B4-(X4)d-B5-(X5)e-B6-B7-(X7)g-B8-(Y2)m (I)

wherein: m and n are independently 0 to 200; B1, B2, B3, B4, B5, B6, B7 and B8 are each independently amino acids selected from Cys, Leu, Ala, Ser, Thr, Met or Gly, at least 6 of the residues B1 through B8 being Cys; a is 6 to 12; c is 8 to 16; d is 2 to 20; e is 4 to 12; and g is 5 to 15; X1, X3, X4, X5, X7, Y1 and Y2 independently represent any amino acid.
In the formula (I), m and n are preferably independently 0 to 10.
In the formula (I), a is preferably 7 to 11.
In the formula (I), c is preferably 10 to 12, more preferably 11.
In the formula (I), d is preferably 4 to 18, more preferably 4 to 16.
In the formula (I), e is preferably 6 to 10, more preferably 9 or 10.
In the formula (I), g is preferably 6 to 12, more preferably 7 to 10.

In the formula (I) preferably B1, B2, B3, B4, B5, B6, B7 and B8 are each independently amino acids selected from Cys, Leu or Ser, at least 7, preferably all 8 of the residues B1 through B8 being Cys. When 7 of the residues B1 through B8 are Cys, it is preferred that: (a) B1 and B3 through B8 are Cys and B2 is other than Cys; (b) B1 through B7 are Cys and B8 is other than Cys, (c) B1 is other than Cys and B2 through B8 are Cys. When 7 of the residues B1 through B8 are Cys, it is preferred that the other residue is Ser, Pro or Leu.

In the formulae (I), preferably the group (X3), comprises the sequence motif ZZXZ, wherein Z is an aliphatic amino acid; and X is any amino acid. In this specification the term “aliphatic amino acid” means an amino acid selected from the group consisting of glycine (G), alanine (A), leucine (L), isoleucine (I), valine (V) and proline (P). More preferably, the group (X3), comprises the sequence motif selected from the group consisting of LLXV, ILXV, ILXL, VLXL and VLXV. Most preferably, the group (X3), comprises the sequence motif VLXV.

Alternatively, in the formulae (II), preferably the group (X3) comprises the sequence motif ZZXZZXZ, wherein Z is an aliphatic amino acid; and X is any amino acid. More preferably, the group (X3) comprises the sequence motif VLZVZXL, wherein Z is an aliphatic amino acid; and X is any amino acid.

In a preferred embodiment, the hydrophobin is obtained from fungi of the genus Trichoderma (particularly Trichoderma harzianum, Trichoderma longibrichiatum, Trichoderma asperellum, Trichoderma Koningiopsis, Trichoderma aggressivum, Trichoderma stromaticum or Trichoderma reesei). Other sources of fungal derived hydrophobins include Cryphonectria parasitica, Ophiostoma ulmi, Gibberella moniliformis, and Magnaporthe griesa. In a preferred embodiment, the hydrophobin is obtained from fungi of the species Trichoderma reesei.

In an especially preferred embodiment, the hydrophobin is the protein HFBII (SEQ ID NO: 70; obtainable from Trichoderma reesei) or a protein having at least 40%, at least 45%, at least 50%, at least 55%, at least 70%, at least 80%, at least 90% or at least 99% sequence identity with SEQ ID NO: 70.

The composition of the invention comprises one or more hydrophobin proteins from about 0.001 to about 5%, preferably from about 0.005 to about 2%, more preferably from about 0.01 to about 1% by weight of the composition.

### VI. Hair Care Compositions

The surface active proteins of the current invention can be used in hair care compositions to provide one or more benefits, including sudsing. The hair care compositions of the present invention can be in different forms. Non-limiting examples of said forms are: shampoos, conditioning shampoos, pet shampoo, leave-in treatments, sprays, liquids, pastes, Newtonian or non-Newtonian fluids, gels, and sols.

The hair care composition preferably comprises at least one surface active protein at a level where upon directed use, promotes one or more benefits without detriment to the hair. In one embodiment of the present invention, said hair care composition comprises between about 0.00001% to about 10% of at least one surface active protein. In another embodiment, said hair care composition comprises between about 0.00005% to about 5% of at least one surface active protein. In another embodiment, said hair care composition comprises between about 0.0001% to about 1% of at least one surface active protein.

In addition to at least one surface active protein, the hair care compositions of the present invention may also include detersive surfactants, aqueous carriers, shampoo gel matrixes, and other additional ingredients.

### Detersive Surfactant

The hair care composition comprises one or more detersive surfactants, which provides cleaning performance to the composition. The one or more detersive surfactants in turn may comprise an anionic surfactant, amphoteric or zwitterionic surfactants, or mixtures thereof. Various examples and descriptions of detersive surfactants are set forth in U.S. Pat. No. 6,649,155; U.S. Patent Application Publication No. 2008/0317698; and U.S. Patent Application Publication No. 2008/0206355, which are incorporated herein by reference in their entirety. The concentration of the detersive surfactant component in the hair care composition should be sufficient to provide the desired cleaning and lather performance, and generally ranges from 2 wt % to about 50 wt %, from about 5 wt % to about 30 wt %, from about 8 wt % to about 25 wt %, from about 10 wt % to about 20 wt %, about 5 wt %, about 10 wt %, about 12 wt %, about 15 wt %, about 17 wt %, about 18 wt %, or about 20 wt %.

Anionic surfactants suitable for use in the compositions are the alkyl and alkyl ether sulfates. Other suitable anionic surfactants are the water-soluble salts of organic, sulfuric acid reaction products. Still other suitable anionic surfactants are the reaction products of fatty acids esterified with isethionic acid and neutralized with sodium hydroxide. Other similar anionic surfactants are described in U.S. Pat. Nos. 2,486,921; 2,486,922; and 2,396,278, which are incorporated herein by reference in their entirety.

Exemplary anionic surfactants for use in the hair care composition include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate and combinations thereof. In a further embodiment, the anionic surfactant is sodium lauryl sulfate or sodium laureth sulfate.

Suitable amphoteric or zwitterionic surfactants for use in the hair care composition herein include those which are known for use in shampoo or other personal care cleansing. Concentrations of such amphoteric surfactants range from about 0.5 wt % to about 20 wt %, and from about 1 wt % to about 10 wt %. Non limiting examples of suitable zwitterionic or amphoteric surfactants are described in U.S. Pat. Nos. 5,104,646 and 5,106,609, which are incorporated herein by reference in their entirety.

Amphoteric detersive surfactants suitable for use in the hair care composition include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate. Exemplary amphoteric detersive surfactants for use in the present hair care composition include cocoamphoacetate, cocoamphodiacetate, lauroamphoacetate, lauroamphodiacetate, and mixtures thereof.

Zwitterionic detersive surfactants suitable for use in the hair care composition include those surfactants broadly described as derivatives of aliphatic quaternaryammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. In another embodiment, zwitterionics such as betaines are selected.

Non limiting examples of other anionic, zwitterionic, amphoteric or optional additional surfactants suitable for use in the hair care composition are described in McCutcheon's, Emulsifiers and Detergents, 1989 Annual, published by M. C. Publishing Co., and U.S. Pat. Nos. 3,929,678, 2,658,072; 2,438,091; 2,528,378, which are incorporated herein by reference in their entirety.

The hair care composition may also comprise a shampoo gel matrix, an aqueous carrier, and other additional ingredients described herein.

### Aqueous Carrier

The hair care composition comprises a first aqueous carrier. The level and species of the carrier are selected according to the compatibility with other components, and other desired characteristic of the product. Accordingly, the formulations of the hair care composition can be in the form of pourable liquids (under ambient conditions). Such compositions will therefore typically comprise a first aqueous carrier, which is present at a level of at least 20 wt %, from about 20 wt % to about 95 wt %, or from about 60 wt % to about 85 wt %. The first aqueous carrier may comprise water, or a miscible mixture of water and organic solvent, and in one aspect may comprise water with minimal or no significant concentrations of organic solvent, except as otherwise incidentally incorporated into the composition as minor ingredients of other components.

The first aqueous carriers useful in the hair care composition include water and water solutions of lower alkyl alcohols and polyhydric alcohols. The lower alkyl alcohols useful herein are monohydric alcohols having 1 to 6 carbons, in one aspect, ethanol and isopropanol. The polyhydric alcohols useful herein include propylene glycol, hexylene glycol, glycerin, and propane diol.

In an embodiment of the present invention, the aqueous carrier is substantially water. In a further embodiment, deionized water may be used. Water from natural sources including mineral cations can also be used, depending on the desired characteristic of the product. Generally, the compositions of the present invention comprise from about 0% to about 99%, in an embodiment from about 50% to about 95%, in a further embodiment from about 70% to about 90%, and in a further embodiment from about 80% to about 90% water.

### Shampoo Gel Matrix

In one embodiment, the hair care composition described herein may comprise a shampoo gel matrix. The shampoo gel matrix comprises (i) from about 0.1% to about 20% of one or more fatty alcohols, alternative from about 0.5% to about 14%, alternatively from about 1% to about 10%, alternatively from about 6% to about 8%, by weight of the shampoo gel matrix; (ii) from about 0.1% to about 10% of one or more shampoo gel matrix surfactants, by weight of the shampoo gel matrix; and (iii) from about 20% to about 95% of an aqueous carrier, alternatively from about 60% to about 85% by weight of the shampoo gel matrix.

The fatty alcohols useful herein are those having from about 10 to about 40 carbon atoms, from about 12 to about 22 carbon atoms, from about 16 to about 22 carbon atoms, or about 16 to about 18 carbon atoms. These fatty alcohols can be straight or branched chain alcohols and can be saturated or unsaturated. Nonlimiting examples of fatty alcohols include, cetyl alcohol, stearyl alcohol, behenyl alcohol, and mixtures thereof. Mixtures of cetyl and stearyl alcohol in a ratio of from about 20:80 to about 80:20 are suitable.

The shampoo gel matrix surfactants may be any of the detersive surfactants described in section “A” herein.

The aqueous carrier may comprise water, or a miscible mixture of water and organic solvent, and in one aspect may comprise water with minimal or no significant concentrations of organic solvent, except as otherwise incidentally incorporated into the composition as minor ingredients of other components.

The aqueous carrier useful herein includes water and water solutions of lower alkyl alcohols and polyhydric alcohols. The lower alkyl alcohols useful herein are monohydric alcohols having 1 to 6 carbons, in one aspect, ethanol and isopropanol. Exemplary polyhydric alcohols useful herein include propylene glycol, hexylene glycol, glycerin, and propane diol.

### Additional Ingredients

### 1. Silicone Conditioning Agent

The compositions of the present invention may contain one or more silicone conditioning agents. Examples of the silicones include dimethicones, dimethiconols, cyclic silicones, methylphenyl polysiloxane, and modified silicones with various functional groups such as amino groups, quaternary ammonium salt groups, aliphatic groups, alcohol groups, carboxylic acid groups, ether groups, sugar or polysaccharide groups, fluorine-modified alkyl groups, alkoxy groups, or combinations of such groups. Such silicones may be soluble or insoluble in the aqueous (or non-aqueous) product carrier. In the case of insoluble liquid silicones, the silicones can be in an emulsified form with droplet size of about 10 nm to about 30 micrometers Other solid or semi-solid conditioning agents may be present in the composition including high melting temperature fatty alcohols, acids, esters, amides or oligomers from unsaturated esters, alcohols, amides. The oligomeric esters may be the result of oligomerization of naturally-occurring unsaturated glyceride esters. Such solid or semi-solid conditioning agents may be added or present as mixtures with organic oils.

### 2. Nonionic Polymers

The hair care composition of the present invention may also further comprise a nonionic polymer. According to an embodiment, the conditioning agent for use in the hair care composition of the present invention may include a polyalkylene glycol polymer. For example, polyalkylene glycols having a molecular weight of more than about 1000 are useful herein. Useful are those having the following general formula (VIII):

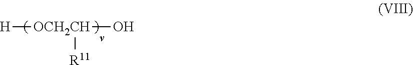

wherein R¹¹ is selected from the group consisting of H, methyl, and mixtures thereof; and v is the number of ethoxy units. The polyalkylene glycols, such as polyethylene glycols, can be included in the hair care compositions of the present invention at a level of from about 0.001 wt. % to about 10 wt. %. In an embodiment, the polyethylene glycol is present in an amount up to about 5 wt. % based on the weight of the composition. Polyethylene glycol polymers useful herein are PEG-2M (also known as Polyox WSR® N-10, which is available from Union Carbide and as PEG-2,000); PEG-5M (also known as Polyox WSR® N-35 and Polyox WSR® N-80, available from Union Carbide and as PEG-5,000 and Polyethylene Glycol 300,000); PEG-7M (also known as Polyox WSR® N-750 available from Union Carbide); PEG-9M (also known as Polyox WSR® N-3333 available from Union Carbide); and PEG-14 M (also known as Polyox WSR® N-3000 available from Union Carbide).

### 3. Organic Conditioning Materials

The conditioning agent of the compositions of the present invention may also comprise at least one organic conditioning material such as oil or wax, either alone or in combination with other conditioning agents, such as the silicones described above. The organic material can be non-polymeric, oligomeric or polymeric. It may be in the form of oil or wax and may be added in the formulation neat or in a pre-emulsified form. Some non-limiting examples of organic conditioning materials include, but are not limited to: i) hydrocarbon oils; ii) polyolefins, iii) fatty esters, iv) fluorinated conditioning compounds, v) fatty alcohols, vi) alkyl glucosides and alkyl glucoside derivatives; vii) quaternary ammonium compounds; viii) polyethylene glycols and polypropylene glycols having a molecular weight of up to about 2,000,000 including those with CTFA names PEG-200, PEG-400, PEG-600, PEG-1000, PEG-2M, PEG-7M, PEG-14M, PEG-45M and mixtures thereof.

### 4. Deposition Aids

The hair care compositions of the present invention may further comprise a deposition aid, such as a cationic polymer. Cationic polymers useful herein are those having an average molecular weight of at least about 5,000, alternatively from about 10,000 to about 10 million, and alternatively from about 100,000 to about 2 million.

Suitable cationic polymers include, for example, copolymers of vinyl monomers having cationic amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone, and vinyl pyrrolidone. Other suitable spacer monomers include vinyl esters, vinyl alcohol (made by hydrolysis of polyvinyl acetate), maleic anhydride, propylene glycol, and ethylene glycol. Other suitable cationic polymers useful herein include, for example, cationic celluloses, cationic starches, and cationic guar gums.

The cationic polymer can be included in the hair care compositions of the present invention at a level of from about 0.001 wt. % to about 10 wt. %. In one embodiment, the cationic polymer is present in an amount up to about 5 wt % based on the weight of the composition.

### 5. Benefit Agents

In an embodiment, the hair care composition further comprises one or more additional benefit agents. The benefit agents comprise a material selected from the group consisting of anti-dandruff agents, anti-fungal agents, anti-itch agents, anti-bacterial agents, anti-microbial agents, moisturization agents, anti-oxidants, vitamins, lipid soluble vitamins, chelants, perfumes, brighteners, enzymes, sensates, attractants, dyes, pigments, bleaches, and mixtures thereof. In one aspect said benefit agent may comprise an anti-dandruff agent. Such anti-dandruff particulate should be physically and chemically compatible with the components of the composition, and should not otherwise unduly impair product stability, aesthetics or performance. According to an embodiment, the hair care composition comprises an anti-dandruff active, which may be an anti-dandruff active particulate. In an embodiment, the anti-dandruff active is selected from the group consisting of: pyridinethione salts; azoles, such as ketoconazole, econazole, and elubiol; selenium sulphide; particulate sulfur; keratolytic agents such as salicylic acid; and mixtures thereof. In an embodiment, the anti-dandruff particulate is a pyridinethione salt. Pyridinethione particulates are suitable particulate anti-dandruff actives. In an embodiment, the anti-dandruff active is a 1-hydroxy-2-pyridinethione salt and is in particulate form. In an embodiment, the concentration of pyridinethione anti-dandruff particulate ranges from about 0.01 wt. % to about 5 wt. %, or from about 0.1 wt. % to about 3 wt. %, or from about 0.1 wt. % to about 2 wt. %. In an embodiment, the pyridinethione salts are those formed from heavy metals such as zinc, tin, cadmium, magnesium, aluminium and zirconium, generally zinc, typically the zinc salt of 1-hydroxy-2-pyridinethione (known as “zinc pyridinethione” or “ZPT”), commonly 1-hydroxy-2-pyridinethione salts in platelet particle form. In an embodiment, the 1-hydroxy-2-pyridinethione salts in platelet particle form have an average particle size of up to about 20 microns, or up to about 5 microns, or up to about 2.5 microns. Salts formed from other cations, such as sodium, may also be suitable. Pyridinethione anti-dandruff actives are described, for example, in U.S. Pat. Nos. 2,809,971; 3,236,733; 3,753,196; 3,761,418; 4,345,080; 4,323,683; 4,379,753; and 4,470,982.

In an embodiment, in addition to the anti-dandruff active selected from polyvalent metal salts of pyrithione, the composition further comprises one or more anti-fungal and/or anti-microbial actives. In an embodiment, the anti-microbial active is selected from the group consisting of: coal tar, sulfur, charcoal, whitfield's ointment, castellani's paint, aluminum chloride, gentian violet, octopirox (piroctone olamine), ciclopirox olamine, undecylenic acid and its metal salts, potassium permanganate, selenium sulphide, sodium thiosulfate, propylene glycol, oil of bitter orange, urea preparations, griseofulvin, 8-hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone, and azoles, and mixtures thereof. In an embodiment, the anti-microbial is selected from the group consisting of: itraconazole, ketoconazole, selenium sulphide, coal tar, and mixtures thereof.

In an embodiment, the azole anti-microbials is an imidazole selected from the group consisting of: benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and mixtures thereof, or the azole anti-microbials is a triazole selected from the group consisting of: terconazole, itraconazole, and mixtures thereof. When present in the hair care composition, the azole anti-microbial active is included in an amount of from about 0.01 wt. % to about 5 wt. %, or from about 0.1 wt. % to about 3 wt. %, or from about 0.3 wt. % to about 2 wt. %. In an embodiment, the azole anti-microbial active is ketoconazole. In an embodiment, the sole anti-microbial active is ketoconazole.

Embodiments of the hair care composition may also comprise a combination of anti-microbial actives. In an embodiment, the combination of anti-microbial active is selected from the group of combinations consisting of: octopirox and zinc pyrithione, pine tar and sulfur, salicylic acid and zinc pyrithione, salicylic acid and elubiol, zinc pyrithione and elubiol, zinc pyrithione and climbasole, octopirox and climbasole, salicylic acid and octopirox, and mixtures thereof.

In an embodiment, the composition comprises an effective amount of a zinc-containing layered material. In an embodiment, the composition comprises from about 0.001 wt. % to about 10 wt. %, or from about 0.01 wt. % to about 7 wt. %, or from about 0.1 wt. % to about 5 wt. % of a zinc-containing layered material, by total weight of the composition.

Zinc-containing layered materials may be those with crystal growth primarily occurring in two dimensions. It is conventional to describe layer structures as not only those in which all the atoms are incorporated in well-defined layers, but also those in which there are ions or molecules between the layers, called gallery ions (A. F. Wells “Structural Inorganic Chemistry” Clarendon Press, 1975). Zinc-containing layered materials (ZLMs) may have zinc incorporated in the layers and/or be components of the gallery ions. The following classes of ZLMs represent relatively common examples of the general category and are not intended to be limiting as to the broader scope of materials which fit this definition.

Many ZLMs occur naturally as minerals. In an embodiment, the ZLM is selected from the group consisting of: hydrozincite (zinc carbonate hydroxide), aurichalcite (zinc copper carbonate hydroxide), rosasite (copper zinc carbonate hydroxide), and mixtures thereof. Related minerals that are zinc-containing may also be included in the composition. Natural ZLMs can also occur wherein anionic layer species such as clay-type minerals (e.g., phyllosilicates) contain ion-exchanged zinc gallery ions. All of these natural materials can also be obtained synthetically or formed in situ in a composition or during a production process.

Another common class of ZLMs, which are often, but not always, synthetic, is layered double hydroxides. In an embodiment, the ZLM is a layered double hydroxide conforming to the formula [M²⁺₁₋ₓM³⁺ₓ(OH)₂]^{x+}A^{m-}_{x/m}.nH₂O wherein some or all of the divalent ions (M²⁺) are zinc ions (Crepaldi, E L, Pava, P C, Tronto, J, Valim, J B J. Colloid Interfac. Sci. 2002, 248, 429-42). Yet another class of ZLMs can be prepared called hydroxy double salts (Morioka, H., Tagaya, H., Karasu, M, Kadokawa, J, Chiba, K Inorg. Chem. 1999, 38, 4211-6). In an embodiment, the ZLM is a hydroxy double salt conforming to the formula [M²⁺₁₋ₓM²⁺₁₊ₓ(OH)_{3(1-y)}]⁺ Aⁿ⁻_{(1=3y)n}.nH₂O where the two metal ions (M²⁺) may be the same or different. If they are the same and represented by zinc, the formula simplifies to [Zn₁₊ₓ(OH)₂]^{2x+} 2x A⁻.nH₂O. This latter formula represents (where x=0.4) materials such as zinc hydroxychloride and zinc hydroxynitrate. In an embodiment, the ZLM is zinc hydroxychloride and/or zinc hydroxynitrate. These are related to hydrozincite as well wherein a divalent anion replace the monovalent anion. These materials can also be formed in situ in a composition or in or during a production process.

In embodiments having a zinc-containing layered material and a pyrithione or polyvalent metal salt of pyrithione, the ratio of zinc-containing layered material to pyrithione or a polyvalent metal salt of pyrithione is from about 5:100 to about 10:1, or from about 2:10 to about 5:1, or from about 1:2 to about 3:1.

The on-scalp deposition of the anti-dandruff active is at least about 1 microgram/cm². The on-scalp deposition of the anti-dandruff active is important in view of ensuring that the anti-dandruff active reaches the scalp where it is able to perform its function. In an embodiment, the deposition of the anti-dandruff active on the scalp is at least about 1.5 microgram/cm², or at least about 2.5 microgram/cm², or at least about 3 microgram/cm², or at least about 4 microgram/cm², or at least about 6 microgram/cm², or at least about 7 microgram/cm², or at least about 8 microgram/cm², or at least about 8 microgram/cm², or at least about 10 microgram/cm². The on-scalp deposition of the anti-dandruff active is measured by having the hair of individuals washed with a composition comprising an anti-dandruff active, for example a composition pursuant to the present invention, by trained a cosmetician according to a conventional washing protocol. The hair is then parted on an area of the scalp to allow an open-ended glass cylinder to be held on the surface while an aliquot of an extraction solution is added and agitated prior to recovery and analytical determination of anti-dandruff active content by conventional methodology, such as HPLC.

### 6. Rheology Modifier/Suspending Agents

In one embodiment, the rinse-off hair care composition comprises a rheology modifier. The rheology modifier increases the substantivity and stability of the composition, improve feel and consumer's use experience (e.g. non-dripping, spreadability, etc). Any suitable rheology modifier can be used. In an embodiment, the hair care composition may comprise from about 0.05% to about 10% of a rheology modifier, in a further embodiment, from about 0.1% to about 10% of a rheology modifier, in yet a further embodiment, from about 0.5% to about 2% of a rheology modifier, in a further embodiment, from about 0.7% to about 2% of a rheology modifier, and in a further embodiment from about 1% to about 1.5% of a rheology modifier. In an embodiment, the rheology modifier may be a polyacrylamide thickener. In an embodiment, the rheology modifier may be a polymeric rheology modifier.

In one embodiment, the rinse-off hair care composition may comprise rheology modifiers that are homopolymers based on acrylic acid, methacrylic acid or other related derivatives, non-limiting examples include polyacrylate, polymethacrylate, polyethylacrylate, and polyacrylamide. In another embodiment, the rheology modifiers may be alkali swellable and hydrophobically-modified alkali swellable acrylic copolymers or methacrylate copolymers non-limiting examples include acrylic acid/acrylonitrogen copolymer, acrylates/steareth-20 itaconate copolymer, acrylates/ceteth-20 itaconate copolymer, acrylates/aminoacrylates copolymer, acrylates/steareth-20 methacrylate copolymer, acrylates/beheneth-25 methacrylate copolymer, acrylates/steareth-20 methacrylate crosspolymer, acrylates/vinylneodecanoate crosspolymer, and acrylates/C10-C30 alkyl acrylate crosspolymer.

In a further embodiment, the rheology modifiers may be crosslinked acrylic polymers, a non-limiting example includes carbomers.

In a father embodiment, the rheology modifiers may be alginic acid-based materials; non-limiting examples include sodium alginate, and alginic acid propylene glycol esters.

In a further embodiment, the rheology modifier may be an associative polymeric thickeners, non-limiting examples include: Hydrophobically modified cellulose derivatives; Hydrophobically modified alkoxylated urethane polymers, nonlimiting example include PEG-150/decyl alcohol/SMDI copolymer, PEG-150/stearyl alcohol/SMDI copolymer, polyurethane-39; Hydrophobically modified, alkali swellable emulsions, non-limiting examples include hydrophobically modified polyacrylates, hydrophobically modified polyacrylic acids, and hydrophobically modified polyacrylamides; hydrophobically modified polyethers wherein these materials may have a hydrophobe that can be selected from cetyl, stearyl, oleayl, and combinations thereof, and a hydrophilic portion of repeating ethylene oxide groups with repeat units from 10-300, in another embodiment from 30-200, in a further embodiment from 40-150. Non-limiting examples of this class include PEG-120-methylglucose dioleate, PEG-(40 or 60) sorbitan tetraoleate, PEG-150 pentaerythrityl tetrastearate, PEG-55 propylene glycol oleate, PEG-150 distearate.

In a further embodiment, the rheology modifier may be cellulose and derivatives; nonlimiting examples include microcrystalline cellulose, carboxymethylcelluloses, hydroxyethylcellulose, hydroxypropylcellulose, hydroxypropylmethylcellulose, methylcellulose, ethylcellulose, nitro cellulose, cellulose sulfate, cellulose powder, and hydrophobically modified celluloses

In an embodiment, the rheology modifier may be a guar and guar derivatives; nonlimiting examples include hydroxypropyl guar, and hydroxypropyl guar hydroxypropyl trimonium chloride.

In an embodiment, the rheology modifier may be polyethylene oxide, polypropylene oxide, and POE-PPO copolymers.

In an embodiment, the rheology modifier may be polyvinylpyrrolidone, crosslinked polyvinylpyrrolidone and derivatives. In a further embodiment, the rheology modifier may be polyvinyalcohol and derivatives.

In a further embodiment, the rheology modifier may be polyethyleneimine and derivatives. In another embodiment, the rheology modifier may be silicas; nonlimiting examples include fumed silica, precipitated silica, and silicone-surface treated silica.

In an embodiment, the rheology modifier may be water-swellable clays non-limiting examples include laponite, bentolite, montmorilonite, smectite, and hectonite.

In an embodiment, the rheology modifier may be gums nonlimiting examples include xanthan gum, guar gum, hydroxypropyl guar gum, Arabia gum, tragacanth, galactan, carob gum, karaya gum, and locust bean gum.

In a further embodiment, the rheology modifier may be, dibenzylidene sorbitol, karaggenan, pectin, agar, quince seed (Cydonia oblonga Mill), starch (from rice, corn, potato, wheat, etc), starch-derivatives (e.g. carboxymethyl starch, methylhydroxypropyl starch), algae extracts, dextran, succinoglucan, and pulleran.

In an embodiment, the composition of the present invention may comprise suspending agents including crystalline suspending agents which can be categorized as acyl derivatives, long chain amine oxides, and mixtures thereof. These suspending agents are described in U.S. Pat. No. 4,741,855. These suspending agents include ethylene glycol esters of fatty acids in one aspect having from about 16 to about 22 carbon atoms. In one aspect, useful suspending agents include ethylene glycol stearates, both mono and distearate, but in one aspect, the distearate containing less than about 7% of the mono stearate. Other suitable suspending agents include alkanol amides of fatty acids, having from about 16 to about 22 carbon atoms, or even about 16 to 18 carbon atoms, examples of which include stearic monoethanolamide, stearic diethanolamide, stearic monoisopropanolamide and stearic monoethanolamide stearate. Other long chain acylhydr derivatives include long chain esters of long chain fatty acids (e.g., stearyl stearate, cetyl palmitate, etc.); long chain esters of long chain alkanol amides (e.g., stearamide diethanolamide distearate, stearamide monoethanolamide stearate); and glyceryl esters (e.g., glyceryl distearate, trihydroxystearin, tribehenin) a commercial example of which is Thixin® R available from Rheox, Inc. Long chain acyl derivatives, ethylene glycol esters of long chain carboxylic acids, long chain amine oxides, and alkanol amides of long chain carboxylic acids in addition to the materials listed above may be used as suspending agents. Other long chain acyl derivatives suitable for use as suspending agents include N,N-dihydrocarbyl amido benzoic acid and soluble salts thereof (e.g., Na, K), particularly N,N-di(hydrogenated) C16, C18 and tallow amido benzoic acid species of this family, which are commercially available from Stepan Company (Northfield, Ill., USA). Examples of suitable long chain amine oxides for use as suspending agents include alkyl dimethyl amine oxides, e.g., stearyl dimethyl amine oxide. Other suitable suspending agents include primary amines having a fatty alkyl moiety having at least about 16 carbon atoms, examples of which include palmitamine or stearamine, and secondary amines having two fatty alkyl moieties each having at least about 12 carbon atoms, examples of which include dipalmitoylamine or di(hydrogenated tallow)amine. Still other suitable suspending agents include di(hydrogenated tallow)phthalic acid amide, and crosslinked maleic anhydride-methyl vinyl ether copolymer.

Non-limiting examples of rheology modifiers include acrylamide/ammonium acrylate copolymer (and)polyisobutene (and) polysorbate 20, acrylamide/sodium acryloyldimethyl taurate copolymer/isohexadecane/polysorbate 80, acrylates copolymer; acrylates/beheneth-25 methacrylate copolymer, acrylates/C10-C30 alkyl acrylate crosspolymer, acrylates/steareth-20 itaconate copolymer, ammonium polyacrylate/Isohexadecane/PEG-40 castor oil, C12-16 alkyl PEG-2 hydroxypropylhydroxyethyl ethylcellulose (HM-EHEC), carbomer, crosslinked polyvinylpyrrolidone (PVP), dibenzylidene sorbitol, hydroxyethyl ethylcellulose (EHEC), hydroxypropyl methylcellulose (HPMC), hydroxypropyl methylcellulose (HPMC), hydroxypropylcellulose (HPC), methylcellulose (MC), methylhydroxyethyl cellulose (MEHEC), PEG-150/decyl alcohol/SMDI copolymer, PEG-150/stearyl alcohol/SMDI copolymer, polyacrylamide/C13-14 isoparaffin/laureth-7; polyacrylate 13/polyisobutene/polysorbate 20; polyacrylate crosspolymer-6, polyamide-3; polyquaternium-37 (and) hydrogenated polydecene (and) trideceth-6, polyurethane-39, sodium acrylate/acryloyldimethyltaurate/dimethylacrylamide, crosspolymer (and) isohexadecane (and) polysorbate 60; sodium polyacrylate. Exemplary commercially-available rheology modifiers include ACULYN™ 28, Klucel™ M CS, Klucel™ H CS, Klucel™ G CS, SYLVACLEAR™ AF1900V, SYLVACLEAR™ PA1200V, Benecel™ E10M, Benecel™ K35M, Optasense™ RMC70, ACULYN™33, ACULYN™46, ACULYN™22, ACULYN™44, Carbopol Ultrez™ 20, Carbopol Ultrez™ 21, Carbopol Ultrez™ 10, Carbopol Ulterez™ 30, Carbopol™ 1342, Carbopol™ 934, Carbopol™ 940, Carbopol™ 950, Carbopol™ 980, and Carbopol™ 981, Acrysol™ 22, Sepigel™ 305, Simulgel™ 600, Sepimax Zen, Simulquat HC 305 and combinations thereof.

### VII. Personal Cleansing Compositions

The surface active proteins of the current invention can be used in personal cleansing compositions to provide one or more benefits, including sudsing. The personal cleansing care compositions of the present invention can be in different forms. Non-limiting examples of said forms are: bar soap, body wash, moisturizing body wash, shower gels, skin cleansers, cleansing milks, in shower body moisturizer, shaving preparations, cleansing compositions used in conjunction with a disposable cleansing cloth, sprays, liquids, pastes, Newtonian or non-Newtonian fluids, gels, and sols.

The personal cleansing composition preferably comprises at least one surface active protein at a level where upon directed use, promotes one or more benefits. In one embodiment of the present invention, said personal cleansing composition comprises between about 0.00001% to about 10% of at least one surface active protein. In another embodiment, said personal cleansing composition comprises between about 0.00005% to about 5% of at least one surface active protein. In another embodiment, said personal cleansing composition comprises between about 0.0001% to about 1% of at least one surface active protein.

In addition to at least one surface active protein, the personal cleansing compositions of the present invention may also include additional ingredients.

Personal cleansing compositions can be multi-phase or single phase. While the components for personal cleansing compositions will be discussed below as being multi-phase for simplicity, the components for each phase could also be used in a single phase. A personal cleansing composition can comprise a cleansing phase and a benefit phase. The cleansing phase and the benefit phase can be blended. The cleansing phase and the benefit phase can also be patterned (e.g. striped and/or marbled).

### Cleansing Phase

A personal cleansing composition can comprise from about 50% to about 99.5%, by weight of the composition, of a cleansing phase. A cleansing phase can include a surfactant. The personal care composition can further comprise from 2% to 20%, by weight of the rinse-off personal care composition, of a surfactant. Surfactants can comprise anionic surfactants, nonionic surfactants, amphoteric surfactants, zwitterionic surfactants, cationic surfactants, or mixtures thereof. The personal care composition can include at least one anionic surfactant. A personal care composition can also comprise, for example, an anionic surfactant, amphoteric surfactant, and a zwitterionic surfactant. Suitable amphoteric or zwitterionic surfactants, for example, can include those described in U.S. Pat. Nos. 5,104,646 and 5,106,609.

Anionic surfactants suitable for use in the cleansing phase of the present compositions include alkyl and alkyl ether sulfates. These materials have the respective formula ROSO₃M and RO(C₂H₄O)ₓSO₃M, wherein R is alkyl or alkenyl of from about 8 to about 24 carbon atoms, wherein x is about 1 to about 10, and M is a water-soluble cation such as ammonium, sodium, potassium, or triethanolamine. The alkyl ether sulfates are typically made as condensation products of ethylene oxide and monohydric alcohols having from about 8 to about 24 carbon atoms. R may have from about 10 to about 18 carbon atoms in both the alkyl and alkyl ether sulfates. The alcohols can be derived from fats, e.g., coconut oil or tallow, or can be synthetic. Lauryl alcohol and straight chain alcohols derived from coconut oil may be used. Such alcohols may be reacted with about 1 or about 3 to about 10 or about 5 molar proportions of ethylene oxide. The resulting mixture of molecular species may have, for example, an average of 3 moles of ethylene oxide per mole of alcohol, is sulfated and neutralized.

Specific examples of alkyl ether sulfates which may be used in the cleansing phase are sodium and ammonium salts of coconut alkyl triethylene glycol ether sulfate; tallow alkyl triethylene glycol ether sulfate, and tallow alkyl hexaoxyethylene sulfate. Suitable alkyl ether sulfates are those comprising a mixture of individual compounds, said mixture having an average alkyl chain length of from about 10 to about 16 carbon atoms and an average degree of ethoxylation of from about 1 to about 4 moles of ethylene oxide.

Other suitable anionic surfactants include water-soluble salts of the organic, sulfuric acid reaction products of the general formula [R¹—SO₃-M], wherein R¹ is chosen from the group consisting of a straight or branched chain, saturated aliphatic hydrocarbon radical having from about 8 to about 24, or about 10 to about 18, carbon atoms; and M is a cation. Suitable examples are the salts of an organic sulfuric acid reaction product of a hydrocarbon of the methane series, including iso-, neo-, ineso-, and n-paraffins, having about 8 to about 24 carbon atoms, preferably about 10 to about 18 carbon atoms and a sulfonating agent, e.g., SO₃, H₂SO₄, oleum, obtained according to known sulfonation methods, including bleaching and hydrolysis. Preferred are alkali metal and ammonium sulfonated C10-18 n-paraffins.

Suitable anionic surfactants for use in the cleansing phase include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, monoethanolamine cocoyl sulfate, sodium tridecyl benzene sulfonate, sodium dodecyl benzene sulfonate, and combinations thereof.

Anionic surfactants with branched alkyl chains such as sodium trideceth sulfate, for example, may be employed. Mixtures of anionic surfactants can also be used.

Amphoteric surfactants can include those that can be broadly described as derivatives of aliphatic secondary and tertiary amines in which an aliphatic radical can be straight or branched chain and wherein an aliphatic substituent can contain from about 8 to about 18 carbon atoms such that one carbon atom can contain an anionic water solubilizing group, e.g., carboxy, sulfonate, sulfate, phosphate, or phosphonate. Examples of compounds falling within this definition can be sodium 3-dodecyl-aminopropionate, sodium 3-dodecylaminopropane sulfonate, sodium lauryl sarcosinate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and products described in U.S. Pat. No. 2,528,378. Other examples of amphoteric surfactants can include sodium lauroamphoacetate, sodium cocoamphoactetate, disodium lauroamphoacetate disodium cocodiamphoacetate, and mixtures thereof. Amphoacetates and diamphoacetates can also be used. Zwitterionic surfactants suitable for use as cleansing surfactant in the structured aqueous cleansing phase include those that are broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate.

Other zwitterionic surfactants suitable for use in the cleansing phase include betaines, including high alkyl betaines such as coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gammacarboxypropyl betaine, and lauryl bis-(2-hydroxypropyl)alpha-carboxyethyl betaine. The sulfobetaines may be represented by coco dimethyl sulfopropyl betaine, stearyl dimethyl sulfopropyl betaine, lauryl dimethyl sulfoethyl betaine, lauryl bis-(2-hydroxyethyl) sulfopropyl betaine and the like; amidobetaines and amidosulfobetaines, wherein the RCONH(CH₂)₃ radical is attached to the nitrogen atom of the betaine are also useful in the present compositions.

Amphoacetates and diamphoacetates can also be used. Non-limiting examples of suitable amphoacetates and diamphoacetates include sodium lauroamphoacetate, sodium cocoamphoactetate, disodium lauroamphoacetate, and disodium cocodiamphoacetate.

Cationic surfactants can also be used in the cleansing phase and may represent from 2% to about 5%, by weight of the cleansing phase.

Suitable nonionic surfactants for use in structured aqueous cleansing phase include condensation products of alkylene oxide groups (hydrophilic in nature) with an organic hydrophobic compound, which may be aliphatic or alkyl aromatic in nature.
Other suitable surfactants or cosurfactants that can generally be used in a cleansing phase for a rinse-off personal care composition are described in McCutcheon's: Detergents and Emulsifiers North American Edition (Allured Publishing Corporation 1947) (1986), McCutcheon's, Functional Materials North American Edition (Allured Publishing Corporation 1973) (1992) and U.S. Pat. No. 3,929,678 (filed Aug. 1, 1974).
The cleansing phase can include a structuring surfactant. Such a structuring surfactant can be included from 2% to about 20%, by weight of the personal care composition; from about 3% to about 15%, by weight of the personal care composition; or from about 5% to about 10%, by weight of the personal care composition. Such a structuring surfactant can include sodium trideceth(n) sulfate, hereinafter STnS, wherein n defines the average moles of ethoxylation. n can range, for example, from about 0 to about 3; n can range from about 0.5 to about 2.7; from about 1.1 to about 2.5; from about 1.8 to about 2.2; or n can be about 2. When n is less than 3, STnS can provide improved stability, improved compatibility of benefit agents within the rinse-off personal care compositions, and/or increased mildness of the rinse-off personal care compositions, such described benefits of STnS are disclosed in U.S. Patent Application Pub. No. 2012/0009285.

The personal care composition can further comprise from about 2% to 20%, by weight of the personal care composition, of a cosurfactant. Cosurfactants can comprise amphoteric surfactants, zwitterionic surfactants, or mixtures thereof. Examples of these types of surfactant are discussed above.

The personal care composition can also comprise a water soluble cationic polymer. The water soluble cationic polymer can be present from about 0.001 to about 3 percent by weight of the personal care composition. The water soluble cationic polymer can also be present from about 0.05 to about 2 percent by weight of the personal care composition. The water soluble cationic polymer can also be present from about 0.1 to about 1 by weight of the personal care composition. The polymer may be in one or more phases as a deposition aid for the benefit agents described herein. Suitable cationic deposition polymers for use in the compositions of the present invention contain, for example, cationic nitrogen-containing moieties such as quaternary ammonium or cationic protonated amino moieties. The cationic protonated amines can be primary, secondary, or tertiary amines depending upon the particular species and the selected pH of the personal care composition.

Nonlimiting examples of cationic deposition polymers for use in compositions include polysaccharide polymers, such as cationic cellulose derivatives. The cationic cellulose polymers can be, for example, the salts of hydroxyethyl cellulose reacted with trimethyl ammonium substituted epoxide, referred to in the industry (CTFA) as Polyquaternium 10 which are available from Amerchol Corp. (Edison, N.J., USA) in their Polymer KG, JR and LR series of polymers. The water soluble cationic polymer comprises, for example, KG-30M. Other suitable cationic deposition polymers include cationic guar gum derivatives, such as guar hydroxypropyltrimonium chloride, specific examples of which include the Jaguar series (preferably Jaguar C-17) commercially available from Rhodia Inc., and N-Hance polymer series commercially available from Ashland.

The water soluble cationic polymer can comprise, for example, a cationic guar. In one example, the cationic guar comprises guar hydroxypropyltrimonium chloride. The guar hydroxypropyltrimonium chloride can comprise, for example, N-Hance™ CG-17 Cationic Guar. The cationic guar can be, for example, selected from a group consisting of N-Hance™ 3196, Jaguar C-500, Jaguar C-17, and a combination thereof. Deposition polymers can have a cationic charge density from about 0.8 meq/g to about 2.0 meq/g or from about 1.0 meq/g to about 1.5 meq/g, or about 0.96 meq/g.

The water soluble cationic polymer can also comprise synthetic polyacrylamides. Examples of suitable synthetic polyacrylamides include polyquaternium 76 and Polymethylene-bis-acrylamide methacrylamido propyltrimethyl ammonium chloride (PAMMAPTAC, AM:MAPTAC ratio 88:12. In one example, the water soluble cationic polymer comprises PAM/MAPTAC.

A cleansing phase of a personal care composition can also include an associative polymer. Such associative polymer can be a crosslinked, alkali swellable, associative polymer comprising acidic monomers and associative monomers with hydrophobic end groups, whereby the associative polymer comprises a percentage hydrophobic modification and a hydrophobic side chain comprising alkyl functional groups. Without intending to be limited by theory, it is believed the acidic monomers can contribute to an ability of the associative polymer to swell in water upon neutralization of acidic groups; and associative monomers anchor the associative polymer into structured surfactant hydrophobic domains, e.g., lamellae, to confer structure to the surfactant phase and keep the associative polymer from collapsing and losing effectiveness in a presence of an electrolyte.

The crosslinked, associative polymer can comprise a percentage hydrophobic modification, which is a mole percentage of monomers expressed as a percentage of a total number of all monomers in a polymer backbone, including both acidic and other non-acidic monomers. Percentage hydrophobic modification of the associative polymer, hereafter % HM, can be determined by the ratio of monomers added during synthesis, or by analytical techniques such as proton nuclear magnetic resonance (NMR). Associative alkyl side chains can comprise, for example, butyl, propyl, stearyl, steareth, cetyl, lauryl, laureth, octyl, behenyl, beheneth, steareth, or other linear, branched, saturated, or unsaturated alkyl or alketh hydrocarbon side chains. The acidic monomer can comprise any acid functional group, for example sulfate, sulfonate, carboxylate, phosphonate, or phosphate or mixtures of acid groups. The acidic monomer can comprise, for example, a carboxylate, alternatively the acidic monomer is an acrylate, including acrylic acid and/or methacrylic acid. The acidic monomer comprises a polymerizable structure, e.g., vinyl functionality. Mixtures of acidic monomers, for example acrylic acid and methacrylic acid monomer mixtures, are useful.

The associative monomer can comprise a hydrophobic end group and a polymerizable component, e.g., vinyl, which can be attached. The hydrophobic end group can be attached to the polymerizable component, hence to the polymer chain, by different means but can be attached by an ether or ester or amide functionality, such as an alkyl acrylate or a vinyl alkanoate monomer. The hydrophobic end group can also be separated from the chain, for example, by an alkoxy ligand such as an alkyl ether. The associative monomer can be, for example, an alkyl ester, an alkyl (meth)acrylate, where (meth)acrylate is understood to mean either methyl acrylate or acrylate, or mixtures of the two.

The hydrophobic end group of the associative polymer can be incompatible with the aqueous phase of the composition and can associate with lathering surfactant hydrophobe components. Without intending to be limited by theory, it is believed that longer alkyl chains of structuring polymer hydrophobe end groups can increase incompatibility with the aqueous phase to enhance structure, whereas somewhat shorter alkyl chains having carbon numbers closely resembling lathering surfactant hydrophobes (e.g., 12 to 14 carbons) or multiples thereof (for bilayers, e.g.) can also be effective. An ideal range of hydrophobic end group carbon numbers combined with an optimal percentage of hydrophobic monomers expressed as a percentage of the polymer backbone can provide increased structure to the lathering, structured surfactant composition at low levels of polymer structurant.

The associative polymer can be Aqupec SER-300 made by Sumitomo Seika of Japan, which is Acrylates/C10-30 alkyl acrylate crosspolymer and comprises stearyl side chains with less than about 1% HM. Other preferred associative polymers can comprise stearyl, octyl, decyl and lauryl side chains. Preferred associative polymers are Aqupec SER-150 (acrylates/C10-30 alkyl acrylates crosspolymer) comprising about C18 (stearyl) side chains and about 0.4% HM, and Aqupec HV-701EDR which comprises about C8 (octyl) side chains and about 3.5% HM. In another example, the associative polymer can be Stabylen 30 manufactured by 3V Sigma S.p.A., which has branched isodecanoate hydrophobic associative side chains.

Other optional additives can be included in the cleansing phase, including for example an emulsifier (e.g., non-ionic emulsifier) and electrolytes. Suitable emulsifiers and electrolytes are described in U.S. patent application Ser. No. 13/157,665.

### Benefit Phase

As noted herein, personal care compositions can include a benefit phase. The composition may comprise from about 0.1% to about 50%, by weight of the composition, of a benefit phase. The benefit phase can be hydrophobic and/or anhydrous. The benefit phase can also be substantially free of or free of surfactant. In particular, the benefit phase can comprise from about 0.1% to about 50%, by weight of the rinse-off personal care composition, of a benefit agent. The benefit phase can include, for example, from about 0.5% to about 20%, by weight of the rinse-off personal care composition, of a benefit agent.

A benefit phase can have a particle size of about 4 to about 500 μm, from about 5 to about 300 μm, from about 6 to about 100 μm, or from about 10 to about 50 μm. The particle size is measured in neat product under a differential interference contrast optical microscope with a 10× objective lens. The particle size distribution is counted manually. All benefit phase particles are assumed as uniform spheres in this application. For irregular shaped benefit phase particles, the longest axis is used as the diameter for the particle size distribution counting. The number weighted average of all lipid particles is defined as the average lipid particle size. This measurement can also be accomplished with a computer algorithm.

A benefit phase can have a viscosity as measured by a standard rheometer, such as a Brookfield R/S plus. A sample of 2.5 mL is measured with a spindle C75-1 at a shear rate of 2 s⁻¹ at 25° C. A benefit phase can generally have a viscosity of about 200 cP to about 15,000 cP. However, it has been discovered that lower viscosity benefit phases (i.e. less than about 2000 cP) can be advantageous for manufacturing as it is easier to blend the benefit phase and the surfactant phase. Thus, for example, the benefit phase has a viscosity of 200 cP to about 1800 cP or from about 300 cP to about 1500 cP.

A benefit agent can include a liquid benefit agent. A liquid benefit agent is considered liquid if that is its natural state at room temperature (i.e. 23° C.). A liquid benefit agent can have a viscosity of less than about 1000 cP, less than about 800 cP, or less than about 600 cP, and can be measured with a standard rheometer.

The liquid benefit agent can have a hydrophobic component. The hydrophobic component can be, for example, a water-dispersible, non-volatile liquid. The water-dispersible, non-volatile liquid benefit agents can have a Vaughn Solubility Parameter (VSP) ranging from about 5 to about 14. Non-limiting examples of hydrophobic benefit materials having VSP values ranging from about 5 to about 14 include the following: Cyclomethicone (5.9), Squalene (6.0), Isopropyl Palmitate (7.8), Isopropyl Myristate (8.0), Castor Oil (8.9), Cholesterol (9.6), Butylene Glycol (13.2), soy bean oil, olive oil (7.87), mineral oil (7.1), and combinations thereof.

Non-limiting examples of glycerides suitable for use as liquid benefit agents herein can include castor oil, safflower oil, corn oil, walnut oil, peanut oil, olive oil, cod liver oil, almond oil, avocado oil, palm oil, sesame oil, soybean oil, vegetable oils, sunflower seed oil, coconut oil, cottonseed oil, jojoba oil, and combinations thereof.

Non-limiting examples of glyceride derivatives suitable for use as liquid benefit agents herein can include cationic derivatives, amino acid derivatives, alkanolamide derivatives, esterified derivatives, ether derivatives, hydrogenated derivatives, and combinations thereof. Non-limiting examples of metathesized oligomers suitable for use as liquid benefit agents herein can include oligomers derived from metathesis of unsaturated polyol esters, for example. Exemplary metathesized unsaturated polyol esters and their starting materials are set forth in U.S. Patent Application U.S. 2009/0220443 A1, which is incorporated herein by reference. The unsaturated polyol ester is an unsaturated ester of glycerol. Sources of unsaturated polyol esters of glycerol include synthesized oil, plant oils, algae oils, bacterial derived oils, and animal oils, combinations of theses, and the like. Representative examples of plant oils include argan oil, canola oil, rapeseed oil, coconut oil, corn oil, cottonseed oil, olive oil, palm oil, peanut oil, safflower oil, sesame oil, soy-bean oil, sunflower oil, high oleoyl soy-bean oil, high oleoyl sunflower oil, linseed oil, palm kernel oil, tung oil, castor oil, high erucic rape oils, Jatropha oil, combinations of theses, and the like. Representative examples of animal oils include fish oil and the like. A representative example of a synthesized oil includes tall oil, which is a byproduct of wood pulp manufacture.

Other examples of unsaturated polyol esters include diesters such as those derived from ethylene glycol or propylene glycol, esters such as those derived from pentaerythritol or dipentaerythritol, or sugar esters such as SEFOSE®. Non-limiting examples of sucrose polyesters suitable for use include SEFOSE® 1618S, SEFOSE® 1618U, SEFOSE® 1618S B6, SEFOSE® 1618U B6, Sefa Cottonate, Sefa C895, Sefa C1095, SEFOSE® 1618S B4.5, all available from The Procter and Gamble Co. of Cincinnati, Ohio. Other examples of suitable natural polyol esters may include but not be limited to sorbitol esters, maltitol esters, sorbitan esters, maltodextrin derived esters, xylitol esters, and other sugar derived esters. The poloyl ester oligomers may also be modified further by partial hydroformylation of the unsaturated functionality to provide one or more OH groups and an increase in the oligomer hydrophilicity.

Non-limiting examples of hydrocarbons suitable for use as liquid benefit agents herein can include carbon chain length of about C6 or higher including alkanes, polyalkanes, olefins, polyolefins and combinations thereof. Non-limiting examples include mineral oil.

Non-limiting examples of glyceride derivatives for use as liquid benefit agents here in can include cationic derivatives, amino acid derivatives, alkanolamide derivatives, esterified derivatives, ether derivatives, hydrogenated or partially hydrogenated oils and their derivatives, and combination thereof.

Non-limiting examples of alkyl esters suitable for use as liquid benefit agents herein can include isopropyl esters of fatty acids and long chain esters of long chain (i.e. C10-C16) fatty acids, non-limiting examples of which can include isopropyl palmitate, isohexyl palmitate and isopropyl myristate.

Non-limiting examples of silicone oils suitable for use as hydrophobic liquid skin benefit agents herein can include dimethicone copolyol, dimethylpolysiloxane, diethylpolysiloxane, mixed C1-C30 alkyl polysiloxanes, phenyl dimethicone, dimethiconol, and combinations thereof. Nonlimiting examples of silicone oils useful herein are described in U.S. Pat. No. 5,011,681. Still other suitable hydrophobic skin benefit agents can include milk triglycerides (e.g., hydroxylated milk glyceride) and polyol fatty acid polyesters.

The benefit agent may also be non-liquid. Some examples of non-liquid benefit agents include hydrocarbons. Non-limiting examples of hydrocarbons suitable for use as non-liquid benefit agents herein can include petrolatum, microcrystalline wax, polyalkanes, polyolefins, and combinations thereof.

Non-limiting examples of glycerides suitable for use as non-liquid benefit agents herein can include plant waxes, animal fats, hydrogenated or partially hydrogenated plant oils, e.g. shea butter, hydrogenated soybean oil, hydrogenated palm, lanolin, lard, and combinations thereof.

Non-limiting examples of metathesized glycerides suitable for use as non-liquid benefit agents herein can include metathesized palm oil, hydrogenated or partially hydrogenated metathesized soybean oil and canola oil, and combinations thereof.

Non-limiting examples of alkyl esters suitable for use as non-liquid benefit agents herein can include isopropyl esters of fatty acids and long chain esters of long chain (i.e. C10-C24) fatty acids, e.g., cetyl ricinoleate, non-limiting examples of which can include cetyl riconoleate and stearyl riconoleate. Other examples can include hexyl laurate, isohexyl laurate, myristyl myristate, decyl oleate, isodecyl oleate, hexadecyl stearate, decyl stearate, isopropyl isostearate, diisopropyl adipate, diisohexyl adipate, dihexyldecyl adipate, diisopropyl sebacate, acyl isononanoate lauryl lactate, myristyl lactate, cetyl lactate, and combinations thereof.

Non-limiting examples of alkenyl esters suitable for use as non-liquid benefit agents can include oleyl myristate, oleyl stearate, oleyl oleate, and combinations thereof.

Non-limiting examples of polyglycerin fatty acid esters suitable for use as non-liquid benefit agents herein can include decaglyceryl distearate, decaglyceryl diisostearate, decaglyceryl monomyriate, decaglyceryl monolaurate, hexaglyceryl monooleate, and combinations thereof.

Non-limiting examples of lanolin and lanolin derivatives suitable for use as non-liquid benefit agents herein can include lanolin, lanolin wax, lanolin alcohols, lanolin fatty acids, isopropyl lanolate, acetylated lanolin, acetylated lanolin alcohols, lanolin alcohol linoleate, lanolin alcohol riconoleate, and combinations thereof.

Non-limiting examples of silicones suitable for use herein can include silicone elastomers.

Other suitable benefit agents are described in U.S. Patent Application Publication No. 2012/0009285.

The benefit phase may also comprise a crystalline hydrophobic ethylene copolymer. The ethylene copolymers are random copolymers and may be present from about 0.01% to about 5% by weight of the personal care composition. The crystalline hydrophobic ethylene copolymer may be present from about 0.05% to about 2% by weight of the personal care composition. As another example, the crystalline hydrophobic ethylene copolymer may be present from about 0.1% to about 1.5% by weight of the personal care composition.

The crystalline hydrophobic ethylene copolymer contains at least 40% ethylene monomer by weight of the crystalline hydrophobic ethylene acrylate copolymer. The crystalline hydrophobic ethylene copolymer can contain from about 40%, 50%, 60%, 70%, 80%, 90%, 95%, 96%, 97%, to about 99%, 98%, 97%, 96%, 95%, 90%, 80%, 70%, 60%, 50%, or any combination thereof to form a range, of ethylene monomer.

In addition, the crystalline hydrophobic ethylene copolymer can comprise an acrylate monomer. The polymer may contain about 1% to about 60%, by weight of the polymer, of an acrylate monomer. The acrylate monomer may be defined by the following formula: (R¹)(R²)C═C(R³)(COOR⁴), wherein, each R¹, R², and R³ is independently H or C₁-C₄-alkyl, in one example H or methyl, in another example two of R¹, R², and R³ are H and the other is H or methyl, in another example R¹, R², and R³ are all H; and R⁴ is C₁-C₂₀-alkyl, or is selected from straight-chain and branched alkyl groups having from 4 to 20, from 6 to 20, from 8 to 20, or from 9 to 20 carbon atoms.

Some examples of suitable crystalline hydrophobic ethylene acrylate copolymers include ethylene:propylheptylacrylate, ethylene:propylheptylacrylate:vinyl acetate, and combinations thereof. A suitable crystalline hydrophobic ethylene acrylate copolymer can include 86.2% ethylene:13.8% propylheptylacrylate; 90.4% ethylene:9.6% propylheptylacrylate; 96% ethylene:4% propylheptylacrylate; or 81.8% ethylene:9.6% propylheptylacrylate:8.6% vinyl acetate.

The crystalline hydrophobic ethylene copolymer can comprise a vinyl actetate monomer. The vinyl acetate monomer may be defined by the following formula: (R¹⁰)(R¹¹)C═C(R⁹)(COR¹²), wherein R⁹ is independently H or C1-C4-alkyl, one of R¹⁰ and R¹¹ is —C(O)R¹³ and the other is H or C₁-C₄-alkyl; and R¹² and R¹³ are each independently —OH or C₁-C₂₀-alkoxy; or R¹² and R¹³ together from an —O— group.

In addition, a crystalline hydrophobic ethylene acrylate copolymer can include a combination of ethylene, propylheptylacrylate, and an additional monomer. This additional monomer can be up to 10%, by weight of the copolymer. This additional monomer can be represented as (R⁵)(R⁶)C═C(R⁷)(OCOR⁸) wherein, each R⁵, R⁶, and R⁷ is independently H or C₁-C₄-alkyl, preferably H or methyl, more preferable two of R⁵, R⁶, and R⁷ are H and the other is H or methyl, in particular R⁵, R⁶, and R⁷ are all H; and R⁸ is C₁-C₂₀-alkyl, preferably C₁-C₉-alkyl, more preferably C₁-C₃-alkyl, specifically either or methyl, and especially methyl. A suitable example of this additional monomer is vinyl acetate.

### Additional Ingredients

Additional ingredients can also be added to the personal care composition for treatment of the skin and/or hair, or to modify the aesthetics of the personal care composition as is the case with perfumes, colorants, dyes or the like. Materials useful in products herein can be categorized or described by their cosmetic and/or therapeutic benefit or their postulated mode of action or function. However, it can be understood that actives and other materials useful herein can, in some instances, provide more than one cosmetic and/or therapeutic benefit or function or operate via more than one mode of action. Therefore, classifications herein can be made for convenience and cannot be intended to limit an ingredient to particularly stated application or applications listed. A precise nature of these additional materials, and levels of incorporation thereof, will depend on the physical form of the composition and the nature of the cleansing operation for which it is to be used. The additional materials can usually be formulated at about 6% or less, about 5% or less, about 4% or less, about 3% or less, about 2% or less, about 1% or less, about 0.5% or less, about 0.25% or less, about 0.1% or less, about 0.01% or less, or about 0.005% or less of the rinse-off personal care composition.

To further improve stability under stressful conditions such as high temperature and vibration, densities of separate phases can be adjusted such that they can be substantially equal. To achieve this, low density microspheres can be added to one or more phases of the rinse-off personal care composition. Examples of rinse-off personal care compositions that comprise low density microspheres are described in a patent application published on May 13, 2004 under U.S. Patent Publication No. 2004/0092415A1 entitled “Striped Liquid Personal Cleansing Compositions Containing A Cleansing Phase and A Separate Phase with Improved Stability,” filed on Oct. 31, 2003 by Focht, et al.

Other non-limiting ingredients that can be used in the personal care composition of the present invention can comprise an optional benefit component that can be selected from the group consisting of thickening agents; preservatives; antimicrobials; fragrances; chelators (e.g. such as those described in U.S. Pat. No. 5,487,884 issued to Bisset, et al.); sequestrants; vitamins (e.g. Retinol); vitamin derivatives (e.g. tocophenyl actetate, niacinamide, panthenol); sunscreens; desquamation actives (e.g. such as those described in U.S. Pat. Nos. 5,681,852 and 5,652,228 issued to Bisset); anti-wrinkle/anti-atrophy actives (e.g. N-acetyl derivatives, thiols, hydroxyl acids, phenol); anti-oxidants (e.g. ascorbic acid derivatives, tocophenol) skin soothing agents/skin healing agents (e.g. panthenoic acid derivatives, aloe vera, allantoin); skin lightening agents (e.g. kojic acid, arbutin, ascorbic acid derivatives) skin tanning agents (e.g. dihydroxyacteone); anti-acne medicaments; essential oils; sensates; pigments; colorants; pearlescent agents; interference pigments (e.g such as those disclosed in U.S. Pat. No. 6,395,691 issued to Liang Sheng Tsaur, U.S. Pat. No. 6,645,511 issued to Aronson, et al., U.S. Pat. No. 6,759,376 issued to Zhang, et al, U.S. Pat. No. 6,780,826 issued to Zhang, et al.) particles (e.g. talc, kolin, mica, smectite clay, cellulose powder, polysiloxane, silicas, carbonates, titanium dioxide, polyethylene beads) hydrophobically modified non-platelet particles (e.g. hydrophobically modified titanium dioxide and other materials described in a commonly owned, patent application published on Aug. 17, 2006 under Publication No. 2006/0182699A, entitled “Personal Care Compositions Containing Hydrophobically Modified Non-platelet particle filed on Feb. 15, 2005 by Taylor, et al.) and mixtures thereof. The multiphase personal care composition can comprise from about 0.1% to about 4%, by weight of the rinse-off personal care composition, of hydrophobically modified titanium dioxide. Other such suitable examples of such skin actives are described in U.S. patent application Ser. No. 13/157,665.

### IX. Shave Preparations

The surface active proteins of the current invention can be used in shave preparations to provide one or more benefits, including sudsing. The shave preparations of the present invention can be in different forms. Non-limiting examples of said forms are: shaving creams, shaving gels, aerosol shaving gels, shaving soaps, aerosol shaving foams, liquids, pastes, Newtonian or non-Newtonian fluids, gels, and sols.

The shave preparation preferably comprises at least one surface active protein at a level where upon directed use, promotes one or more benefits. In one embodiment of the present invention, said shave preparation comprises between about 0.00001% to about 10% of at least one surface active protein. In another embodiment, said shave preparation comprises between about 0.00005% to about 5% of at least one surface active protein. In another embodiment, said shave preparation comprises between about 0.0001% to about 1% of at least one surface active protein.

In addition to at least one surface active protein, the shave preparations of the present invention may also include lathering surfactants, carriers, adjunt ingredients, and other additional ingredients.

### Lathering Surfactants

The shave preparations can comprise one or more lathering surfactants and a carrier such at water, at a total level of from about 60% to about 99.99%. A lathering surfactant defined herein as surfactant, which when combined with water and mechanically agitated generates a foam or lather. Preferably, these surfactants or combinations of surfactants should be mild, which means that these surfactants provide sufficient cleansing or detersive benefits but do not overly dry the skin or hair while still being able to produce a lather.

A wide variety of lathering surfactants are useful herein and include those selected from the group consisting of anionic lathering surfactants, nonionic lather surfactants, amphoteric lathering surfactants, and mixtures thereof. Generally, the lathering surfactants are fairly water soluble. When used in the composition, at least about 4% of the lathering surfactants have a HLB value greater than about ten. Examples of such surfactants are found in and U.S. Pat. No. 5,624,666. Cationic surfactants can also be used as optional components, provided they do not negatively impact the overall lathering characteristics of the required lathering surfactants.

Concentrations of these surfactant are from about 10% to about 20%, alternatively from about 5% to about 25%, and alternatively from 2% to about 60% by weight of the composition.

Anionic lathering surfactants useful in the compositions of the present invention are disclosed in McCutcheon's, Detergents and Emulsifiers, North American edition (1986), published by allured Publishing Corporation; McCutcheon's, Functional Materials, North American Edition (1992); and U.S. Pat. No. 3,929,678. A wide variety of anionic lathering surfactants are useful herein. Non-limiting examples of anionic lathering surfactants include those selected from the group consisting of sarcosinates, sulfates, sulfonates, isethionates, taurates, phosphates, lactylates, glutamates, and mixtures thereof.

Other anionic materials useful herein are soaps (i.e., alkali metal salts, e.g., sodium or potassium salts) of fatty acids, typically having from about 8 to about 24 carbon atoms, preferably from about 10 to about 20 carbon atoms, monoalkyl, dialkyl, and trialkylphosphate salts, alkanoyl sarcosinates corresponding to the formula RCON(CH₃)CH₂CH₂CO₂M wherein R is alkyl or alkenyl of about 10 to about 20 carbon atoms, and M is a water-soluble cation such as ammonium, sodium, potassium and alkanolamine (e.g., triethanolamine). Also useful are taurates which are based on taurine, which is also known as 2-aminoethanesulfonic acid, and glutamates, especially those having carbon chains between C8 and C16.

Non-limiting examples of preferred anionic lathering surfactants useful herein include those selected from the group consisting of sodium lauryl sulfate, ammonium lauryl sulfate, ammonium laureth sulfate, sodium laureth sulfate, sodium trideceth sulfate, ammonium cetyl sulfate, sodium cetyl sulfate, ammonium cocoyl isethionate, sodium lauroyl isethionate, sodium lauroyl lactylate, triethanolamine lauroyl lactylate, sodium caproyl lactylate, sodium lauroyl sarcosinate, sodium myristoyl sarcosinate, sodium cocoyl sarcosinate, sodium lauroyl methyl taurate, sodium cocoyl methyl taurate, sodium lauroyl glutamate, sodium myristoyl glutamate, and sodium cocoyl glutamate and mixtures thereof.

Suitable amphoteric or zwitterionic detersive surfactants for use in the compositions herein include those which are known for use in hair care or other personal care cleansing. Concentration of such amphoteric detersive surfactants is from about 1% to about 10%, alternatively from about 0.5% to about 20% by weight of the composition. Non-limiting examples of suitable zwitterionic or amphoteric surfactants are described in U.S. Pat. Nos. 5,104,646 and 5,106,609.

Nonionic lathering surfactants for use in the compositions of the present invention are disclosed in McCutcheon's, Detergents and Emulsifiers, North American edition (1986), published by allured Publishing Corporation; and McCutcheon's, Functional Materials, North American Edition (1992); both of which are incorporated by reference herein in their entirety. Nonionic lathering surfactants useful herein include those selected from the group consisting of alkyl glucosides, alkyl polyglucosides, polyhydroxy fatty acid amides, alkoxylated fatty acid esters, lathering sucrose esters, amine oxides, and mixtures thereof.

Other examples of nonionic surfactants include amine oxides. Amine oxides correspond to the general formula R¹R²R³NO, wherein R¹ contains an alkyl, alkenyl or monohydroxy alkyl radical of from about 8 to about 18 carbon atoms, from 0 to about 10 ethylene oxide moieties, and from 0 to about 1 glyceryl moiety, and R² and R³ contain from about 1 to about 3 carbon atoms and from 0 to about 1 hydroxy group, e.g., methyl, ethyl, propyl, hydroxyethyl, or hydroxypropyl radicals. Examples of amine oxides suitable for use in this invention include dimethyl-dodecylamine oxide, oleyldi(2-hydroxyethyl) amine oxide, dimethyloctylamine oxide, dimethyl-decylamine oxide, dimethyl-tetradecylamine oxide, 3,6,9-trioxaheptadecyldiethylamine oxide, di(2-hydroxyethyl)-tetradecylamine oxide, 2-dodecoxyethyldimethylamine oxide, 3-dodecoxy-2-hydroxypropyldi(3-hydroxypropyl)amine oxide, dimethylhexadecylamine oxide.

Preferred lathering surfactants for use herein are the following, wherein the anionic lathering surfactant is selected from the group consisting of ammonium lauroyl sarcosinate, sodium trideceth sulfate, sodium lauroyl sarcosinate, sodium myristoyl sarcosinate, ammonium laureth sulfate, sodium laureth sulfate, ammonium lauryl sulfate, sodium lauryl sulfate, ammonium cocoyl isethionate, sodium cocoyl isethionate, sodium lauroyl isethionate, sodium cetyl sulfate, sodium lauroyl lactylate, triethanolamine lauroyl lactylate, and mixtures thereof; wherein the nonionic lathering surfactant is selected from the group consisting of lauramine oxide, cocoamine oxide, decyl polyglucose, lauryl polyglucose, sucrose cocoate, C₁₂₋₁₄ glucosamides, sucrose laurate, and mixtures thereof; and wherein the amphoteric lathering surfactant is selected from the group consisting of disodium lauroamphodiacetate, sodium lauroamphoacetate, cetyl dimethyl betaine, cocoamidopropyl betaine, cocoamidopropyl hydroxy sultaine, and mixtures thereof.

One suitable lathering surfactant is a polyglyceryl fatty ester. In one embodiment the polyglyceryl fatty ester surfactant has the formula:

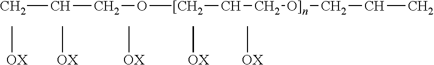

wherein n is 1 to 10, and X is a hydrogen atom or a long chain acyl group derived from a C12-22 fatty acid or an N-fatty acyl-neutral amino acid, provided that at least one X is a long chain acyl group and no more than three X's are long chain acyl groups. In one embodiment, the polyglyceryl fatty ester surfactant is selected from the group consisting of: polyglyceryl-10 oleate, polyglyceryl-6 stearate, polyglyceryl-10 stearate, polyglyceryl-8 dipalmitate, polyglyceryl-10 dipalmitate, polyglyceryl-10 behenate, and polyglyceryl-12 trilaurate.

### Carriers

The shave preparation of the present invention can also comprise a carrier. In one embodiment the carrier comprises water. The carrier is preferably dermatologically acceptable, meaning that the carrier is suitable for topical application to the keratinous tissue, has good aesthetic properties, is compatible with the actives of the present invention and any other components, and will not cause any safety or toxicity concerns. In one embodiment, the shave preparation comprises from about 50% to about 99.99%, preferably from about 60% to about 99.9%, more preferably from about 70% to about 98%, and even more preferably from about 80% to about 95% of the carrier by weight of the composition.

### Adjunct Ingredients

### 1. Lubricants

In one embodiment, said shave preparation comprises at least one lubricant selected from: a lubricious water soluble polymer; a water insoluble particle, a hydrogel forming polymer, and a mixture thereof.

The lubricious water soluble polymer will generally have a molecular weight greater between about 300,000 and 15,000,000 daltons, preferably more than about one million daltons, and will include a sufficient number of hydrophilic moieties or substituents on the polymer chain to render the polymer water soluble. The polymer may be a homopolymer, copolymer or terpolymer. Examples of suitable lubricious water soluble polymers include polyethylene oxide, polyvinylpyrrolidone, and polyacrylamide. A preferred lubricious water soluble polymer comprises polyethylene oxide, and more particularly a polyethylene oxide with a molecular weight of about 0.5 to about 5 million daltons. Examples of suitable polyethylene oxides PEG-23M, PEG-45M, and PEG-90M. The lubricious water soluble polymer can be at a level of about 0.005% to about 3%, preferably about 0.01% to about 1%, by weight.

The water insoluble particles may include inorganic particles or organic polymer particles. Examples of inorganic particles include titanium dioxide, silicas, silicates and glass beads, with glass beads being preferred. Examples of organic polymer particles include polytetrafluoroethylene particles, polyethylene particles, polypropylene particles, polyurethane particles, polyamide particles, or mixtures of two or more of such particles.

The hydrogel-forming polymer is a highly hydrophilic polymer that, in water, forms organized three-dimensional domains of approximately nanometer scale. The hydrogel-forming polymer generally has a molecular weight greater than about one million daltons (although lower molecular weights are possible) and typically is at least partially or lightly crosslinked and may be at least partially water insoluble, but it also includes a sufficient number of hydrophilic moieties so as to enable the polymer to trap or bind a substantial amount of water within the polymer matrix and thereby form three-dimensional domains. Generally, the hydrogel-forming polymer will be included in the shaving composition in an amount of about 0.0005% to about 3%, or about 0.001% to about 0.5%, or about 0.002% to about 0.1%, by weight.

Examples of suitable hydrogel-forming polymers include a polyacrylic acid or polymethacrylic acid partially esterified with a polyhydric alcohol; hydrophilic polyurethanes; lightly crosslinked polyethylene oxide; lightly crosslinked polyvinyl alcohol; lightly crosslinked polyacrylamide; hydrophobically modified hydroxyalkyl cellulose; hydroxyethyl methacrylate; and crosslinked hyaluronic acid. A preferred hydrogel-forming polymer comprises polyacrylic acid partially esterified (e.g., about 40% to 60%, preferably about 50%, esterified) with glycerin. Such a polymer includes glyceryl acrylate/acrylic acid copolymer. Glyceryl acrylate/acrylic acid copolymer is highly hydrophilic, has a molecular weight greater than 1 million daltons and generally includes a polyacrylic acid backbone partially esterified (typically about 50% esterified) with glycerin. It is believed that the glyceryl acrylate/acrylic acid copolymer forms a clathrate that holds water, which, upon release, supplies lubrication and moisturization to the skin. It has been found that shave gel compositions that include the glyceryl acrylate/acrylic acid copolymer have improved gel structure and reduced coefficient of friction (i.e., increased lubricity). See e.g. U.S. 2006/00257349 at ¶10.

The term “water dispersible”, as used herein, means that a substance is either substantially dispersible or soluble in water. The water dispersible surface active agent is preferably one that is capable of forming a lather, such as one or more of the optional lathering surfactants described in section 5 below (including but not limited to a soap, an interrupted soap, a detergent, an anionic surfactant, a non-ionic surfactant or a mixture of one or more of these.)

### 2. Polar Solvents

In one embodiment, the carrier comprises a polar solvent. The level of polar solvent can be from about 1% to about 20%, or from about 5% to about 10%. Polar solvents useful herein include polyhydric alcohols such as, 3-butylene glycol, propane diol, ethylene glycol, diethylene glycol, sorbitol, and other sugars which are in liquid form at ambient temperature glycerin, sorbitol, propylene glycol, butylene glycol, pentylene glycol, hexylene glycol, ethoxylated glucose, 1,2-hexane diol, hexanetriol, dipropylene glycol, erythritol, trehalose, diglycerin, xylitol, maltitol, maltose, glucose, fructose, sodium chondroitin sulfate, sodium hyaluronate, sodium adenosine phosphate, sodium lactate, pyrrolidone carbonate, glucosamine, cyclodextrin, and mixtures thereof. Polyols such as those containing from 2 to about 6 carbon atoms and from 2 to about 6 hydroxy groups are preferred (e.g., 1,3-propanediol, ethylene glycol, glycerin, and 1,2-propanediol) can also be used. The most preferred are Butylene, Pentylene or Hexylene Glycol and mixtures there of.

Without intending to be bound by theory, it is believed that the addition of one or more, polar solvents, allows for reduction in the viscosity and improvement in the clarity of the shave preparation while maintaining good lubrication.

### 3. Salycylic Acid

The shave preparation of the present invention may comprise a salicylic acid compound, its esters, its salts, or combinations thereof. In the compositions of the present invention, the salicylic acid compound preferably comprises from about 0.1% to about 5%, preferably from about 0.2% to about 2%, and more preferably from about 0.5% to about 2%, by weight of the composition, of salicylic acid.

### 4. Other Adjunct Ingredients

The compositions of the present invention may contain a variety of other ingredients that are conventionally used in given product types provided that they do not unacceptably alter the benefits of the invention. These ingredients should be included in a safe and effective amount for a shave preparation for application to skin.

The CTFA Cosmetic Ingredient Handbook, Second Edition (1992) describes a wide variety of nonlimiting cosmetic and pharmaceutical ingredients commonly used in the skin care industry, which are suitable for use in the compositions of the present invention. Examples of these ingredient classes include: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, fatty alcohols and fatty acids, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents, skin-conditioning agents, skin soothing and/or healing agents and derivatives, skin treating agents, thickeners, and vitamins and derivatives thereof.

Additional non-limiting examples of additional suitable skin treatment actives are included in U.S. 2003/0082219 in Section I (i.e. hexamidine, zinc oxide, and niacinamide); U.S. Pat. No. 5,665,339 at Section D (i.e. coolants, skin conditioning agents, sunscreens and pigments, and medicaments); and US 2005/0019356 (i.e. desquamation actives, anti-acne actives, chelators, flavonoids, and antimicrobial and antifungal actives). Other useful optional ingredients include: Anti-Wrinkle Actives and/or Anti-Atrophy Actives; Anti-Oxidants and/or Racial Scavengers; Anti-Inflammatory Agents; Anti-Cellulite Agents; Tanning Actives; Skin Lightening Agents; Sunscreen Actives; Water Soluble Vitamins; particulates; and combinations thereof.

The shave preparation of the present invention is a non-aerosol composition. In one embodiment, the shave preparation is free or substantially free of a volatile post-foaming agent.

a. Conditioning Agents

The compositions of the present invention may comprise a conditioning agent selected from the group consisting of humectants, moisturizers, or skin conditioners, each can be present at a level of from about 0.01% to about 40%, more preferably from about 0.1% to about 30%, and even more preferably from about 0.5% to about 15% by weight of the composition. These materials include, but are not limited to, guanidine; urea; glycolic acid and glycolate salts (e.g. ammonium and quaternary alkyl ammonium); lactic acid and lactate salts (e.g., ammonium and quaternary alkyl ammonium); aloe vera in any of its variety of forms (e.g., aloe vera gel); polyhydroxy compounds such as sorbitol, mannitol, glycerol, hexanetriol, butanetriol, propylene glycol, butylene glycol, hexylene glycol and the like; polyethylene glycols; sugars (e.g., melibiose) and starches; sugar and starch derivatives (e.g., alkoxylated glucose, fructose, sucrose, etc.); hyaluronic acid; lactamide monoethanolamine; acetamide monoethanolamine; sucrose polyester; petrolatum; and mixtures thereof.

Suitable moisturizers, also referred to in the present invention as humectants, include urea, guanidine, glycolic acid and glycolate salts (e.g. ammonium and quaternary alkyl ammonium), lactic acid and lactate salts (e.g. ammonium and quaternary alkyl ammonium), aloe vera in any of its variety of forms (e.g. aloe vera gel), polyhydroxy alcohols (such as sorbitol, glycerol, hexanetriol, propylene glycol, hexylene glycol and the like), polyethylene glycol, sugars and starches, sugar and starch derivatives (e.g. alkoxylated glucose), hyaluronic acid, lactamide monoethanolamine, acetamide monoethanolamine, and mixtures thereof.

b. Thickening Agents (Including Thickeners and Gelling Agents)

The compositions of the present invention can comprise one or more thickening agents, preferably from about 0.05% to about 10%, more preferably from about 0.1% to about 5%, and even more preferably from about 0.25% to about 4%, by weight of the composition. Nonlimiting classes of thickening agents include those selected from the group consisting of: Carboxylic Acid Polymers (crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol); crosslinked polyacrylate polymers (including both cationic and nonionic polymers, such as described in U.S. Pat. Nos. 5,100,660; 4,849,484; 4,835,206; 4,628,078; 4,599,379, and EP 228,868); polymeric sulfonic acid (such as copolymers of acryloyldimethyltaurate and vinylpyrrolidone) and hydrophobically modified polymeric sulfonic acid (such as crosspolymers of acryloyldimethyltaurate and beheneth-25 methacrylate); polyacrylamide polymers (such as nonionic polyacrylamide polymers including substituted branched or unbranched polymers such as polyacrylamide and isoparaffin and laureth-7 and multi-block copolymers of acrylamides and substituted acrylamides with acrylic acids and substituted acrylic acids); polysaccharides (nonlimiting examples of polysaccharide gelling agents include those selected from the group consisting of cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof); gums (i.e. gum agents such as acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof); and crystalline, hydroxyl-containing fatty acids, fatty esters or fatty waxes (such as microfibrous bacterial cellulose structurants as disclosed in U.S. Pat. No. 6,967,027 to Heux et al.; U.S. Pat. No. 5,207,826 to Westland et al.; U.S. Pat. No. 4,487,634 to Turbak et al.; U.S. Pat. No. 4,373,702 to Turbak et al. and U.S. Pat. No. 4,863,565 to Johnson et al., U.S. Patent Publ. No. 2007/0027108 to Yang et al.)

### Compositional pH

The shave preparation of the present invention preferably has a pH of less than about 9, more preferably less than about 7. In one embodiment the composition has a pH of less than about 5, or less than about 4. In one preferred embodiment the composition has a pH range of from about 2.5 to about 4.5 Suitable lathering surfactants for use at pH levels below about 4 can be selected from the group consisting of alkyl sulfonates, pareth sulfonates, sulfobetaines, alkylhydroxysultaines, alkylglucosides and mixtures thereof.

### Low Surfactant Levels

In one aspect, the personal care composition of the present invention comprises relatively low levels of surfactant. Due to the sudsing and surface activity of the surface active proteins of the present invention, suitable cleaning performance can be obtained with relatively lower levels of total surfactant. In such aspect, the personal care composition can preferably comprise from about 0.01% to about 2%, preferably from about 0.01% to about 1.5%, preferably from about 0.01% to about 1%, preferably from about 0.01% to about 0.05%, preferably from about 0.01% to about 0.2%, by weight of the composition, of total surfactant.

### X. EXAMPLES

The following examples are provided to further illustrate the present invention and are not to be construed as limitations of the present invention, as many variations of the present invention are possible without departing from its spirit or scope.

### Example 1a—Production of Thermoactinomyces vulgaris BslA_ClassIII

A codon optimized gene (SEQ ID NO: 25) encoding for a Thermoactinomyces vulgaris BslA_ClassIII variant, without the N-terminal signal peptide but including an N-terminal His-tag and a TEV protease cleavage site (SEQ ID NO: 26), is designed and synthesized. After gene synthesis, the protein is expressed and purified by Genscript (Piscataway, N.J.). In brief, the complete synthetic gene sequence is subcloned into a pET30a vector for heterologous expression. Escherichia coli BL21 (DE3) cells are transformed with the recombinant plasmid and a single colony is inoculated into TB medium containing kanamycin. Isopropyl β-D-1-thiogalactopyranoside (IPTG) is added (final concentration 0.1 mM) to induce protein expression and the culture is incubated at 37° C. for 4 hrs. Cells are harvested by centrifugation and the pellets are lysed by sonication. After centrifugation, the supernatant is collected and the protein is purified by one-step purification using a nickel affinity column and standard protocols known in the art. The protein is stored in a buffer containing 50 mM Tris-HCl, 150 mM NaCl, and 10% Glycerol at pH 8.0. The final protein concentration is 0.193 mg/mL as determined by Bradford protein assay with BSA as a standard (ThermoFisher, catalog #23236).

### Example 1b—Production of Bacillus licheniformis BslA_ClassIV

A codon optimized gene (SEQ ID NO: 27) encoding for a Bacillus licheniformis BslA_ClassIV variant, without the N-terminal signal peptide but including an N-terminal His-tag and a TEV protease cleavage site (SEQ ID NO: 28), is designed and synthesized. After gene synthesis, the protein is expressed and purified by Genscript (Piscataway, N.J.). In brief, the complete synthetic gene sequence is subcloned into a pET30a vector for heterologous expression. Escherichia coli BL21 Star (DE3) cells are transformed with the recombinant plasmid and a single colony was inoculated into TB medium containing kanamycin. When OD₆₀₀ reached 4, isopropyl β-D-1-thiogalactopyranoside (IPTG) is added (final concentration 0.1 mM) to induce protein expression and the culture is incubated at 15° C. for 16 h. Cells are harvested by centrifugation and the pellets are lysed by sonication. After centrifugation, the pellet is dissolved using urea and the protein is purified by one-step purification using a nickel affinity column and standard protocols known in the art. The protein is refolded and stored in buffer containing 1×PBS and 0.5% Sodium Lauroyl Sarcosine at pH 7.4. The final protein concentration is 0.60 mg/mL as determined by Bradford protein assay with BSA as a standard (ThermoFisher, catalog #23236).

### Example 1c—Production of Bacillus subtilis YuaB_classIa

A codon optimized gene (SEQ ID NO: 29) encoding for a Bacillus subtilis YuaB_classIa variant, without the N-terminal signal peptide but including an N-terminal His-tag and a TEV protease cleavage site (SEQ ID NO: 30), is designed and synthesized. After gene synthesis, the protein is expressed and purified by Genscript (Piscataway, N.J.). In brief, the complete synthetic gene sequence is subcloned into a pET30a vector for heterologous expression. Escherichia coli BL21 (DE3) cells are transformed with the recombinant plasmid and a single colony is inoculated into TB medium containing the proper kanamycin. Isopropyl β-D-1-thiogalactopyranoside (IPTG) is added (final concentration 0.1 mM) to induce protein expression and the culture is incubated at 37° C. for 4 hrs. Cells are harvested by centrifugation and the pellets are lysed by sonication. After centrifugation, the supernatant was collected and the protein is purified by one-step purification using a nickel affinity column and standard protocols known in the art. The protein is stored in a buffer containing 50 mM Tris-HCl, 150 mM NaCl, and 10% Glycerol at pH 8.0. The final protein concentration is 6.10 mg/mL as determined by Bradford protein assay with BSA as a standard (ThermoFisher, catalog #23236).

### Example 1d—Production of Bacillus velezensis YweA_classII

A codon optimized gene (SEQ ID NO: 31) encoding for a Bacillus velezensis YweA_classII variant, without the N-terminal signal peptide but including an N-terminal His-tag and a TEV protease cleavage site (SEQ ID NO: 32), is designed and synthesized. After gene synthesis, the protein is expressed and purified by Genscript (Piscataway, N.J.). In brief, the complete synthetic gene sequence is subcloned into a pET30a vector for heterologous expression. Escherichia coli BL21 (DE3) cells are transformed with the recombinant plasmid and a single colony is inoculated into TB medium containing the proper kanamycin. Isopropyl β-D-1-thiogalactopyranoside (IPTG) is added (final concentration 0.1 mM) to induce protein expression and the culture is incubated at 37° C. for 4 h. Cells are harvested by centrifugation and the pellets are lysed by sonication. After centrifugation, the supernatant is collected and the protein is purified by one-step purification using a nickel affinity column and standard protocols known in the art. The protein is stored in a buffer containing 50 mM Tris-HCl, 150 mM NaCl, and 10% Glycerol at pH 8.0. The final protein concentration is 4.50 mg/mL as determined by Bradford protein assay with BSA as a standard (ThermoFisher, catalog #23236).

### Example 2a—Production of Streptomyces coelicolor A3(2) ChpE

Chaplin ChpE without the N-terminal signal peptide (SEQ ID NO: 49) is chemically synthesized (Genscript; Piscataway, N.J.) by solid phase peptide synthesis using standard protocols known in the art to obtain a material with 92.9 w % purity as determined by HPLC analysis.

### Example 2b—Production of Streptomyces coelicolor A3(2) ChpF

A codon optimized gene (SEQ ID NO: 54) encoding for Streptomyces coelicolor A3(2) ChpF (SEQ ID NO: 50) is designed and synthesized. After synthesis, the gene is subcloned into a modified pET28a vector for heterologous expression of a ChpF variant including an additional N-terminal region containing a His-tag, a MBP tag, and a TEV protease cleavage site (SEQ ID NO: 55). The protein is expressed and purified by Genscript (Piscataway, N.J.). In brief, Escherichia coli BL21 (DE3) cells are transformed with the recombinant plasmid and a single colony is inoculated into TB medium containing the proper kanamycin. Cultures are incubated at 15° C. for 16 h at 200 rpm and isopropyl β-D-1-thiogalactopyranoside (IPTG) was added (final concentration 1 mM) to induce protein expression. Cells are harvested by centrifugation and the pellets are lysed by sonication. After centrifugation, the supernatant is collected and the protein is purified by one-step purification using a nickel affinity column and standard protocols known in the art. The protein is stored in a buffer containing 50 mM Tris-HCl, 150 mM NaCl, and 10% Glycerol at pH 8.0. The final protein concentration is 1.30 mg/mL as determined by Bradford protein assay with BSA as a standard (ThermoFisher, catalog #23236).

### Example 3a—Production of Engystomops pustulosus Ep-Rsn2

A codon optimized gene (SEQ ID NO: 66) encoding for an Engystomops pustulosus Ep-Rsn2 variant, including an N-terminal His-tag and a TEV protease cleavage site (SEQ ID NO: 67), is designed and synthesized and the protein is expressed and purified by Genscript (Piscataway, N.J.). In brief, the complete synthetic gene sequence is subcloned into a pET30a vector for heterologous expression. Escherichia coli BL21 (DE3) cells are transformed with the recombinant plasmid and a single colony was inoculated into TB medium containing the proper kanamycin. Isopropyl β-D-1-thiogalactopyranoside (IPTG) is added (final concentration 0.1 mM) to induce protein expression and the culture is incubated at 15° C. and 200 rpm for 16 hrs. Cells are harvested by centrifugation and the pellet is lysed by sonication. After centrifugation, the supernatant is collected and the protein is purified by one-step purification using a nickel affinity column and standard protocols known in the art. The protein is stored in a buffer containing 50 mM Tris-HCl, 150 mM NaCl, and 10% Glycerol at pH 8.0. The final protein concentration is 1.25 mg/mL as determined by Bradford protein assay with BSA as a standard (ThermoFisher, catalog #23236).

### Example 3b—Production of Leptodactylus vastus Lv-Rsn1

A codon optimized gene (SEQ ID NO: 68) encoding for a Leptodactylus vastus Lv-Rsn1 variant, including an N-terminal His-tag, and a TEV protease cleavage site (SEQ ID NO: 69), is designed and synthesized and the protein is expressed and purified by Genscript (Piscataway, N.J.). In brief, the complete synthetic gene sequence is subcloned into a pPICZalpha-A vector for heterologous expression. The linearized construct is then transformed into Pichia pastoris X-33 and the insert of the target gene is confirmed by PCR analysis. Four colonies are inoculated in BMGY for protein expression. When OD₆₀₀ reached 3, the cells are harvested and re-suspended in BMMY media. Methanol is added to a final concentration of 1% every 24 hours for 4 days. After centrifugation, the supernatants are collected and analyzed by SDS-PAGE. The protein is purified by two-step purification using Ni column and SP Sepharose column and standard protocols known in the art. The protein is stored in a buffer containing 50 mM Tris-HCl, 150 mM NaCl, and 10% Glycerol at pH 8.0. The final protein concentration is 50 μg/mL as determined by Micro-Bradford protein assay with BSA as a standard (ThermoFisher, catalog #23236).

### Example 4—Clarifying Shampoo

Table 1 exemplifies a clarifying shampoo comprising one or more surface active proteins according to the invention.

TABLE 1

| Ingredient | (wt./wt.) % |
|---|---|
| Sodium lauryl sulfate | 2.0 to 9.5 |
| Sodium laureth-3 sulfate | 2.0 to 6.0 |
| Hydroxypropyl Methylcellulose (Methocel E50) | 0.25 |
| Tetrasodium EDTA tetrathydrate | 0.16 |
| Sodium benzoate | 0.25 |
| Cocamidopropyl Betaine | 2.0 to 5.0 |
| Sodium xylenesulfonate | 0.21 |
| Ethylene Diamine Disuccinic Acid Trisodium Salt | 0.26 |
| Kathon | 0.0033 |
| Perfume | 0.65 |
| Surface active protein | 0.1 to 5.0 |
| Water | Q.S to 100 |

### Example 5—Conditioning Shampoo

Table 2 exemplifies a conditioning shampoo comprising one or more surface active proteins according to the invention.

TABLE 2

| Ingredient | (wt./wt.)% |
|---|---|
| Water Purified | Q.S to 100 |
| Sodium Laureth 3 Sulfate 28% solution | 2.0 to 22.0 |
| Sodium Lauryl Sulfate 29% solution | 2.0 to 34.5 |
| Laureth-4 | 0.9 |
| Dimethicone 330M cps | 0.5 |
| Glycol Distearate | 1.5 |
| Polyquaternium-6 | 0.32 |
| Sodium Benzoate | 0.27 |
| Citric acid 50% Solution | 0.52 |
| Methylchloroisothiazolinone/methylisothiazolinone | 0.035 |
| Sodium chloride | 1.66 |
| Fragrance | 0.65 |
| Glycol Distearate | 1.5 |
| Surface active protein | 0.1 to 5.0 |

### Example 6—Personal Care Cleanser

Table 3 exemplifies personal care cleasers comprising one or more surface active proteins according to the invention.

TABLE 3

| | Rinse off composition | | |
|---|---|---|---|
| Ingredient, (wt./wt.) % | A | B | C |
| Distilled water | Q.S. | Q.S. | Q.S. |
| Sodium Tridecyl Ether | 2.0 to 13.0 | 2.0 to 12.5 | 2.0 to 12.0 |
| Sulfate - 2 mol | | | |
| Cocamidopropyl Betaine | 2.0 to 8.5 | 2.0 to 8.5 | 2.0 to 8.0 |
| TDA 3-Ethoxylated Tridecyl | 1.34 | 1.29 | 1.23 |
| Alcohol | | | |
| Sodium Chloride | 4.58 | 4.44 | 4.22 |
| Guar Hydroxypropyl- | 0.44 | 0.42 | 0.40 |
| trimonium Chloride | | | |
| (median charge density of | | | |
| about 0.96 meq/g) | | | |
| Xanthan Gum | 0.20 | 0.19 | 0.18 |
| Acrylates/C10-30 Alkyl | 0.03 | 0.03 | 0.03 |
| Acrylate Crosspolymer | | | |
| Citric Acid Anhydrous | 0.10 | 0.10 | 0.10 |
| (Global) | | | |
| Perfume | 1.25 | 1.25 | 1.25 |
| Soybean oil | 2.0 | 5.0 | 10.0 |
| Surface active protein | 0.1 to 5.0 | 0.1 to 5.0 | 0.1 to 5.0 |

### Example 7—Skin Cleansing Compositions

Table 4 exemplifies skin cleansing compositions comprising one or more surface active proteins according to the invention.

TABLE 4

| | | | | Facial | Facial | | |
|---|---|---|---|---|---|---|---|
| | Facial Cleanser-Synthetic | | | Cleanser- | Cleanser- | Thermal | |
| Ingredient (w/w %) | Surfactant | | | Cream | Clear Gel | Scrub | Bar Soap |
| Water | qs | qs | qs | qs | qs | 0 | 12.000 |
| PEG-4 | | | | | | qs | |
| Disodium EDTA | 0.120 | 0.120 | 0.120 | 0.100 | 0.050 | | |
| Tetrasodium EDTA | | | | | | | 0.050 |
| PPG 15 Stearyl Ether | | | | 4.000 | | | |
| PPG-30 | | | | 0.250 | | | |
| Distearyldimonium | | | | 1.500 | | 0.8 | |
| Chloride | | | | | | | |
| Salicylic Acid | | | 2.000 | 1.800 | | | |
| Lauric Acid | 2.000 | | | | 2.000 | | |
| Sodium Tallowate | | | | | | | 68.080 |
| Magnesium Sulfate | | | | | | 20.000 | |
| Behentrimonium | | | | | | 1.780 | |
| Chloride, Stearyl | | | | | | | |
| Alcohol | | | | | | | |
| Sodium Palm Kernelate | | | | | | | 17.020 |
| Myristic Acid | | | | | 6.000 | | |
| Cetyl Betaine^{[3]} | | | | 6.667 | | | |
| Sodium Myristoyl | 5-10.00 | 5-10% | 5-10% | | | | |
| Sarcosinate | | | | | | | |
| Dioleoylethyl | | | | | | 2.000 | |
| Hydroxyethylmonium | | | | | | | |
| Methosulfate | | | | | | | |
| Glycol Distearate (and) | 2.000 | | | | | | |
| Sodium Laureth Sulfate | | | | | | | |
| (and) Cocamide MEA | | | | | | | |
| (and) Laureth 10 ^{[4]} | | | | | | | |
| Sodium | 10-20% | 10-20% | 10-20% | | | | |
| Lauroamphoacetate | | | | | | | |
| Sodium Trideceth | 4-10% | 4-10% | 4-10% | | | | |
| Sulfate | | | | | | | |
| Lauramidopropyl | | | | | 10.00-20.00 | | |
| Betain | | | | | | | |
| Sodium Lauryl Sulfate | | | | 3.571 | | | |
| PEG/PPG-300/55 | | | | | | 3.000 | |
| Copolymer | | | | | | | |
| PEG-100 | 0.200 | 0.200 | 0.200 | | | | |
| PEG 7M | | | | | 0.100 | | |
| Glycerin | 2.000 | 2.000 | 2.000 | 3.000 | 8.000 | | |
| Sorbitol (70%) | 2.857 | 2.857 | 2.857 | | | | |
| Citric Acid | 0.625 | 0.500 | 0.133 | | | | |
| Triethanolamine | | | | | 7.500 | | |
| Sodium Chloride | | | | | | | 0.600 |
| Coconut Acid | | | | | | | 1.000 |
| Steareth-2 | | | | 0.250 | | | |
| Steareth-21 | | | | 0.500 | | | |
| Stearyl Alcohol | | | | 2.880 | | 1.3 | |
| Behenyl Alcohol | | | | 0.320 | | | |
| Cetyl Alcohol | | | | 0.800 | | 0.5 | |
| Polyquaternium-10 | 0.125 | | | | | | |
| (JR400) | | | | | | | |
| Titanium Dioxide | | | | | | | 0.250 |
| Propylparaben | | | | | | 0.1 | |
| Methylparaben | | | | | | 0.2 | |
| Kathon | 0.030 | | 0.030 | | | | |
| DMDM (70%) and | | 0.400 | | | 0.400 | | |
| IPBC | | | | | | | |
| PEG-120 Methyl | | 0.500 | | | | | |
| Glucose Dioleate | | | | | | | |
| PEG-200 | 3.500 | | | | | | |
| Hydrogenated/PEG-7 | | | | | | | |
| Glyceryl | | | | | | | |
| Cocoate/Water (Antil | | | | | | | |
| 200) | | | | | | | |
| Hydroxypropylcellulose | | | | | | 0.25 | |
| Hydroxyethylcellulose^{[5]} | | | | | 1.000 | | |
| Acrylates Co-polymer^{[1]} | 3.90-4.667 | 3.90-4.667 | 3.90-4.667 | | | | |
| Poly(oxy-1,2- | | | 0.625 | | | | |
| ethanediyl), alpha- | | | | | | | |
| hydro-omega-hydroxy-, | | | | | | | |
| ether with methyl D- | | | | | | | |
| glucopyranoside (4:1), | | | | | | | |
| tri-(9Z)-9- | | | | | | | |
| octadecenoate ^{[2]} | | | | | | | |
| BHT | 0.040 | 0.040 | | | | | |
| Fragrance | 0.300 | 0.250 | 0.300 | 0.200 | 0.200 | 0.150 | 1.000 |
| Surface active protein | 0.1 to 5.0 | 0.1 to 5.0 | 0.1 to 5.0 | 0.1 to 5.0 | 0.1 to 5.0 | 0.1 to 5.0 | 0.1 to 5.0 |
| Making Instructions | Mix water, beads, surfactant. Heat | | | Phase A: | Phase A: | Add all | The bar soap |
| | to 75 C.. When solution is | | | Add water | Add all | ingredients | can be |
| | homogenous, add remaining | | | soluble | other | except | prepared in a |
| | ingredients, except fragrance, | | | ingredients | ingredients | beads | conventional |
| | preservative, and surface active | | | & beads. | except | while | manner |
| | protein. Cool to 40 C., add | | | Heat to | fragrance, | heating | where soap |
| | fragrance, preservative, and | | | 65 C.. | preservative, | to 95C. | noodles are |
| | surface active protein. | | | Phase B: | and | Mix until | mixed in an |
| | | | | Add oil | surface | homogenous. | amalgamator |
| | | | | soluble | active | Add | with the |
| | | | | ingredients. | protein. | beads. | perfume, |
| | | | | Heat to | Add | | titanium |
| | | | | 75 C.. Pour | beads. | | dioxide, and |
| | | | | Phase B | Heat to | | beads. The |
| | | | | into Phase | 65 C.. | | amalgamated |
| | | | | A. Cool to | Phase B: | | mixture can |
| | | | | 45 C. & add | Create | | then be |
| | | | | fragrance, | fatty acid | | refined with |
| | | | | preservative, | premix. | | conventional |
| | | | | and | Heat to | | bar soap |
| | | | | surface | 75 C.. Pour | | refining |
| | | | | active | Phase B | | equipment |
| | | | | protein. | into Phase | | such as a mill |
| | | | | | A. Cool | | or refining |
| | | | | | to 45 C. | | plodder. The |
| | | | | | and add | | refined |
| | | | | | fragrance, | | mixture can |
| | | | | | preservative, | | then be |
| | | | | | and | | extruded and |
| | | | | | surface | | cut into |
| | | | | | active | | billets via a |
| | | | | | protein. | | standard |
| | | | | | | | vacuum |
| | | | | | | | plodder, and |
| | | | | | | | bar |
| | | | | | | | cutter. The |
| | | | | | | | resulting |
| | | | | | | | billets can |
| | | | | | | | then be |
| | | | | | | | stamped into |
| | | | | | | | the desired |
| | | | | | | | shape using a |
| | | | | | | | conventional |
| | | | | | | | soap press. |

^{[1]}Carbopol Aqua SF-1 ® from Noveon ™
^{[2]} Glucamate LT from Lubrizol ™
^{[3]}Mackam CET from Rhodia ™
^{[4]} Euperlan PK 810 AM from Cognis ™
[5]Natursol 250 HRR from Hercules Inc.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition comprising: a) one or more surface active proteins selected from the group consisting of Class III BslA proteins, Class IV BslA proteins, chaplin proteins, ranaspumins, latherins, and mixtures thereof and b) a surfactant.

2. The composition according to claim 1 wherein the Class III BslA protein has at least 50% amino acid identity to a wild-type protein: Thermoactinomyces vulgaris BslA (SEQ ID NO: 6).

3. The composition according to claim 1 wherein the Class IV BslA protein has at least 50% amino acid identity to at least one wild-type protein sequence selected from the group consisting of: B. licheniformis BslA (SEQ ID NO: 7, SEQ ID NO: 8, SEQ ID NO: 9, SEQ ID NO: 10, and SEQ ID NO: 11), B. glycinifermentans BslA (SEQ ID NO: 12, SEQ ID NO: 13, SEQ ID NO: 14, and SEQ ID NO: 15), B. sonorensis BslA (SEQ ID NO: 16), B. paralicheniformis BslA (SEQ ID NO: 17, and SEQ ID NO: 18), and Bacillus sp. BslA (SEQ ID NO: 19, SEQ ID NO: 20, SEQ ID NO: 21, and SEQ ID NO: 22).

4. The composition according to claim 3 wherein the Class IV BslA protein has at least 90% amino acid identity to B. licheniformis BslA (SEQ ID NO: 7).

5. The composition according to claim 1, wherein the chaplin proteins have at least 50% amino acid identity to at least one protein selected from the group consisting of SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, or SEQ ID NO: 55.

6. The composition according to claim 5 wherein the chaplin proteins have at least 50% amino acid identity to at least one wild-type protein selected from the group consisting of: Streptomyces coelicolor ChpD (SEQ ID NO: 48), Streptomyces coelicolor ChpE (SEQ ID NO: 49), Streptomyces coelicolor ChpF (SEQ ID NO: 50), Streptomyces coelicolor ChpG (SEQ ID NO: 51), and Streptomyces coelicolor ChpH (SEQ ID NO: 52).

7. The composition according to claim 6 wherein the chaplin proteins have at least 50% amino acid identity to at least one wild-type protein selected from the group consisting of Streptomyces coelicolor ChpE (SEQ ID NO: 49) and Streptomyces coelicolor ChpF (SEQ ID NO: 50).

8. The composition according to claim 1, wherein the ranaspumins have at least 40% amino acid identity to at least one wild-type protein selected from the group consisting of: Engystomops pustulosus Ep-Rsn2 (SEQ ID NO: 56), Leptodactylus vastus Lv-Rsn1 (SEQ ID NO: 57), Leptodactylus fuscus Lf-Rsn1 (SEQ ID NO: 58), and Bufo gargarizans Bg-Rsn (SEQ ID NO: 59).

9. The composition according to claim 8, wherein the ranaspumins have at least 40% amino acid identity to at least one wild-type protein selected from the group consisting of Engystomops pustulosus Ep-Rsn2 (SEQ ID NO: 56) and Leptodactylus vastus Lv-Rsn1 (SEQ ID NO: 57).

10. The composition according to claim 1, further comprising one or more co-proteins, wherein the co-proteins have at least 40% amino acid identity to at least one wild-type protein selected from the group consisting of: Engystomops pustulosus Ep-Rsn1 (SEQ ID NO: 60), Engystomops pustulosus Ep-Rsn3 (SEQ ID NO: 61), Engystomops pustulosus Ep-Rsn4 (SEQ ID NO: 62), Engystomops pustulosus Ep-Rsn5 (SEQ ID NO: 63), and Engystomops pustulosus Ep-Rsn6 (SEQ ID NO: 64); and mixtures thereof.

11. The composition according to claim 10, further comprising one or more co-proteins, wherein the co-proteins have at least 40% amino acid identity to at least one wild-type protein selected from the group consisting of Engystomops pustulosus Ep-Rsn3 (SEQ ID NO: 61) and Engystomops pustulosus Ep-Rsn5 (SEQ ID NO: 63).

12. The composition according to claim 1, further comprising one or more carbohydrates selected from the group consisting of O-glycan, N-glycan, and mixtures thereof.

13. The composition according to claim 1, wherein the latherins have at least 40% amino acid identity to Equus caballus latherin (SEQ ID NO: 65).

14. The composition according to claim 1, wherein the surface active proteins are present in an amount from 0.0001 wt % to 5 wt % by weight of the composition based on active protein.

15. The composition according to claim 14 wherein the surface active proteins are present in an amount from 0.01 wt % to 1 wt %, by weight of the composition based on active protein.

16. The composition according to claim 1, wherein the surfactant is present in an amount of from 2 wt % to 30 wt %, by weight of the composition.

17. The composition according to claim 16, wherein the surfactant is present in an amount of from 3 wt % to 25 wt %, by weight of the composition.

18. The composition according to claim 1, wherein the surfactant is present in an amount of from about 0.01% to about 2%, by weight of the composition.

19. The composition according to claim 18, wherein the surfactant is present in an amount from about 0.01% to about 1.5%, by weight of the composition.

20. The composition according to claim 18, wherein the surfactant is present in an amount from about 0.01% to about 1%, by weight of the composition.


SEQUENCE LISTING (70 sequences)

SEQ ID NO: 1 - protein, 181 aa, Bacillus subtilis
MKRKLLSSLAISALSLGLLVSAPTASFAAESTSTKAHTESTMRTQSTASLFATITGASKTEWSFSDIELTYRPNTLLSLGVMEFTLPSGFTANTKDTLNGNALRTTQILNNGKTVRVPLALDLLGAGEFKLKLNNKTLPAAGTYTFRAENKSLSIGNKFYAEASIDVAKRSTPPTQPCGCN

SEQ ID NO: 2 - protein, 181 aa, Bacillus amyloliquefaciens DSM 7
MKMKHKFFSTVMASLFGLVLLLSLPTASFAAESSSTVHEPEMSTKATATLFAKYTGASQQEWSFSDIELTYRPNTILSLGVMEFTLPSGFTATTKDTVNGHALRERQILNNGKTVRLPLNIDLIGAAEFKLSLNNKTLPAAGTYKFRAENKSLSIGSKFYAEDTIVVQKRSTPPTQPCNCK

SEQ ID NO: 3 - protein, 179 aa, Bacillus licheniformis DSM 3
MKRMYRSKLSILAVSLVMMASIFLPSFQASAQTTKTESVYRPAASASLYSVITGASKQEWSFSDIELTYRPNSILALGTVEFTLPSGFSATTKDTVNGRALTTGQILNNGKTVRLPLTIDLLGIAEFKLVLANKTLPAAGKYTFRAENRVLGLGSTFYAESSIEVQKRATPPTQPCNCK

SEQ ID NO: 4 - protein, 177 aa, Bacillus pumilus
MKKTWTMIMMGMLTLVMALSVPIAASAEGATQEGKASTNARPAELYAKITGTSKQEWSFSDIELTYRPNSVLSLGAIEFTLPAGFQATTKDIFNGKALKDSYILNSGKTVRIPARLDLLGISQFKLQLSHKVLPAAGTYTFRAENRALSIGSKFYAEDTLDIQTRPVVVTPPDPCGC

SEQ ID NO: 5 - protein, 154 aa, Bacillus subtilis
MLKRTSFVSSLFISSAVLLSILLPSGQAHAQSASIEAKTVNSTKEWTISDIEVTYKPNAVLSLGAVEFQFPDGFHATTRDSVNGRTLKETQILNDGKTVRLPLTLDLLGASEFDLVMVRKTLPRAGTYTIKGDVVNGLGIGSFYAETQLVIDPR

SEQ ID NO: 6 - protein, 163 aa, Thermoactinomyces vulgaris
MRLRMVWLSVIVGLSWMLVGWTNPPAPADAEVPSKWQAAQGKALTVRLEPSKNKQEAVSDVVITYKPDVVLAYGRAKFHLPKGFSAVAGDLINGQPLTADYIQNGGQTVTLPFGVDIGAMRIFELRLVGKKLPSAGQYKFRAEYWGIGIGIYNTAEAVLELRN

SEQ ID NO: 7 - protein, 157 aa, Bacillus licheniformis
MLKRKFIGKIGVGLLTSAALFSFILPTEEASATFYRNAPTLHIETVDSNKEWTTSDIEVTYKPNFFVGSSYVEFNFPYRFHANTRDSLNGRTLNYTQILNDGQTVRVPVYAFSSSEFKLVMVRKTLPNAGTHRITAELQKFGRNYNHAEATVDILPR

SEQ ID NO: 8 - protein, 160 aa, Bacillus licheniformis ATCC 14580
MKTNHFRGPIFCPPPFKPAPKPQPPQKPQPDNPQPDNPTQKPVLEAKTINSNKEWSTSDIEITYRPNAFVGSSYVEFHFPYRFHASTRDTLNGKTLDYTQILNDGQTVRVPVYALSSSEFKLVMVRKTLPNAGTHRITAELQQNGKNINHAETTLEIVPR

SEQ ID NO: 9 - protein, 157 aa, Bacillus licheniformis ATCC 14580
MLKRKFIGKIGVGLLTSAALFSFILPTEEASATFYRNAPTLHVETVDSNKEWTTSDIEVTYKPNFFVGSSYVEFNFPYRFHANTRDSLNGRTLNYTQILNDGQTVRVPVYAFSSSEFKLVMVRKTLPNAGTHRITAELQKFGRNYNHAEATVDILPR

SEQ ID NO: 10 - protein, 194 aa, Bacillus licheniformis CG-B52
MLKRKFIRKISVGLLASAALFSFIIPTQGANAQEVKTNHFRGPIFCPPPFKPAPKPQPPQKPQPDNPQPDNPTQKPVLEAKTINSNKEWSTSDIEITYRPNAFVGSSYVEFHFPYRFHASTRDTLNGKTLDYTQILNDGQTVRVPVYALSSSEFKLVMVRKTLPNAGTHRITAELQQNGKNINHAETTLEIVPR

SEQ ID NO: 11 - protein, 194 aa, Bacillus licheniformis S 16
MLKRKFIRKISVGLLASAALFSFIIPTQGANAQEVKTNHFRGPIFCPPPFKPSPKPQPPQKPQPDNPQPDNPTQKPVLEAKTINSNKEWSTSDIEITYRPNSFVGSSYVEFHFPYRFHASTRDTLNGKTLDYTQILNDGQTVRVPVYALSSSEFKLVMVRKTLPNAGTHRITAELQQNGKNINHAETTLEIVPR

SEQ ID NO: 12 - protein, 192 aa, Bacillus glycinifermentans
MLKRKIISKISIGLLTSAALFSFILPTNEANAQEVNTSHFRGPIFCPPPFKPKPQPPQNPKPDNPLPDNPTQKPVLEVKAVNSNKEWTTSDIEITYKPNTFVGTSYVEFNFPYRFHASTRDTLNGKTLDYTQILNDGQTVRVPVYALSSSEFKLVMVRKTLPNAGTHRITAELQQNGKNINHAEATLEIVPR

SEQ ID NO: 13 - protein, 157 aa, Bacillus glycinifermentans
MLKRKFIGKMSVGLLASAALFSFILPSQEANAAFYRSEPALHVETTQSNKEWTTSDIEVTYKPNMFVGASFVEFNFPYRFHADTRDSLNGRTLNYTQILNDGQTVRVPVYAFSSSQFKLVMVRKTLPNAGTHRITAELQKWGRNYGHAETMVEIAPR

SEQ ID NO: 14 - protein, 192 aa, Bacillus glycinifermentans
MLKRKIISKISIGLLTSAALFSFILPTNEANAQEVSTSHFRGPIFCPPPFKPKPQPPQYPKPDNPLPDNPTQKPVLEVKAVNSNKEWTTSDIEITYKPNTFVGTSYVEFNFPYRFHASTRDTLNGKTLDYTQILNDGQTVRVPVYALSSSEFKLVMVRKTLPNAGTHRITAELQQNGKNINHAEATLEIVPR

SEQ ID NO: 15 - protein, 157 aa, Bacillus glycinifermentans
MLKRKFIGKMRVGLLASAALFSFILPSQEANAAFYRSEPALHVETTQSNKEWTTSDIEVTYKPNMFVGASYVEFNFPYRFHANTRDSLNGRTLNYTQILNDGQTVRVPVYAFSSSQFKLVMVRKTLPNAGTHRITAELQKWGRNYGHAETTVEIAPR

SEQ ID NO: 16 - protein, 157 aa, Bacillus sonorensis
MLKRNIISKISIGLLTSAALFSFILPSQEAHATFFRSEPTLHVETVDSNKEWTTSDIEVTYKPNFFVGASYVEFNFPYRFHANTRDSLNGRTLNYTQILNDGQTVRVPVYAFSSSQFKLVMVRKTLPNAGTHRVTAELQKFGRHYHHAEATVEIAPR

SEQ ID NO: 17 - protein, 178 aa, Bacillus paralicheniformis ATCC  9945a
MMNLFFPFSSFLTTFIRRGKYMLKRKFIGKMGVGLLTSAALFSFILPTEEASATFFRNAPTLHVETVDSNKEWTTSDIEVTYKPNSFVGASYVEFNFPYRFHANTRDSLNGRTLNYTQILNDGQTVRVPVYAFSSSAFKLVMVRKTLPNAGTHRVTAELQKFGRNYNHAEATVDILPR

SEQ ID NO: 18 - protein, 194 aa, Bacillus paralicheniformis ATCC 9945a
MLKRKFIRKISVALLASAALFSFIIPTQGANAQEVKTNHFRGPIFCPPPFKPWPKPQPPQKPQPDYPQPDNPTQKPVLEAKTINSNKEWSTSDIEITYKPNTFVGSSYVEFHFPYRFHASTRDTLNGKTLDYTQILNDGQTVRVPVYALSSSEFKLVMVRKTLPNAGTHRITAELQQNGKNINHAETTLEIVPR

SEQ ID NO: 19 - protein, 157 aa, Bacillus sp. NRRL B-41282
MFKRKFVGKLGVGLLSSAALLSFILPAQEASATFFRSAPTLHVETVDSNKEWTTSDIEVTYKPNSFVGSSYVEFNFPYRFHANTRDSLNGRTLNYTQILNDGQTVRVPVYAFSSSQFKLVMVRKTLPNAGTHKITAELQKFGRNYNHAETTVEIVPR

SEQ ID NO: 20 - protein, 185 aa, Bacillus sp. NRRL B-41282
MFKRKFIRKLSVGLLTSAALFSFILPTSGASAQEVNTKHFRGPIFCPPPHWPKKPQPKQPDDPTKQPVLEAKTINSNKEWTTSDIEITYKPNTFVGSSYVEFHFPYRFHASTRDTLNGKALEYTQILNDGQTVRVPVYALSSSEFKLVMIRKTLPNAGTHRITAELHQNGKNINHAETTLEIVPR

SEQ ID NO: 21 - protein, 157 aa, Bacillus sp. NRRL B-41327
MLKRKFIGKMGVGLLTSAALFSFILPTEEASATFYRNAPTLHVETVDSNKEWTTSDIEVTYKPNSFVGSSYVEFNFPYRFHANTRDSLNGRTLSYTQILNDGQTVRVPVYAFSSSEFKLVMVRKTLPNAGTHRITAELQKFGRNYNHAETTVDILPR

SEQ ID NO: 22 - protein, 194 aa, Bacillus sp. NRRL B-41327
MLKRKFIRKISVGLLASAALFSFIIPTQGANAQEVKTNHFRGPIFCPPPFKPSPKPQPPQKPQPDNPQSDNPTQKPVLEAKTINSNKEWSTSDIEITYRPNSFVGSSYVEFHFPYRFHASTRDTLNGKTLDYTQILNDGQTVRVPVYALSSSEFKLVMVRKTLPNAGTHRITAELQQNGKNINHAETTLEIVPR

SEQ ID NO: 23 - protein, 110 aa, Artificial Sequence
  Concensus amino acid sequence
  misc_feature (6)..(6): Xaa can be any naturally occurring amino acid
  misc_feature (12)..(12): Xaa can be any naturally occurring amino acid
  misc_feature (15)..(15): Xaa can be any naturally occurring amino acid
  misc_feature (18)..(18): Xaa can be any naturally occurring amino acid
  misc_feature (22)..(22): Xaa can be any naturally occurring amino acid
  misc_feature (24)..(24): Xaa can be any naturally occurring amino acid
  misc_feature (28)..(28): Xaa can be any naturally occurring amino acid
  misc_feature (36)..(36): Xaa can be any naturally occurring amino acid
  misc_feature (40)..(40): Xaa can be any naturally occurring amino acid
  misc_feature (44)..(45): Xaa can be any naturally occurring amino acid
  misc_feature (47)..(47): Xaa can be any naturally occurring amino acid
  misc_feature (65)..(65): Xaa can be any naturally occurring amino acid
  misc_feature (69)..(69): Xaa can be any naturally occurring amino acid
  misc_feature (75)..(75): Xaa can be any naturally occurring amino acid
  misc_feature (86)..(87): Xaa can be any naturally occurring amino acid
  misc_feature (92)..(94): Xaa can be any naturally occurring amino acid
  misc_feature (96)..(99): Xaa can be any naturally occurring amino acid
  misc_feature (103)..(106): Xaa can be any naturally occurring amino acid
  misc_feature (108)..(108): Xaa can be any naturally occurring amino acid
SNKEWXTSDIEXTYXPNXFVGXSXVEFXFPYRFHAXTRDXLNGXXLXYTQILNDGQTVRVPVYAXSSSXFKLVMXRKTLPNAGTHXXTAELXXXGXXXXHAEXXXXIXPR

SEQ ID NO: 24 - protein, 141 aa, Thermoactinomyces vulgaris
NPPAPADAEVPSKWQAAQGKALTVRLEPSKNKQEAVSDVVITYKPDVVLAYGRAKFHLPKGFSAVAGDLINGQPLTADYIQNGGQTVTLPFGVDIGAMRIFELRLVGKKLPSAGQYKFRAEYWGIGIGIYNTAEAVLELRN

SEQ ID NO: 25 - DNA, 423 bp, Artificial Sequence
  Sequence optimized synthetic gene
aacccgccggcgccggcggatgcggaagtgccgagcaagtggcaagcggcgcagggtaaagcgctgaccgtgcgtctggagccgagcaagaacaaacaggaagcggttagcgacgtggttattacctacaagccggatgtggttctggcgtatggccgtgcgaagttccacctgccgaaaggttttagcgcggttgcgggcgacctgattaacggtcaaccgctgaccgcggattacatccagaacggtggccaaaccgtgaccctgccgttcggcgttgatattggtgcgatgcgtatctttgagctgcgtctggtgggcaagaaactgccgagcgcgggccagtacaaattccgtgcggaatattggggtatcggcattggtatctataacaccgcggaggcggttctggaactgcgtaac

SEQ ID NO: 26 - protein, 155 aa, Thermoactinomyces vulgaris
MHHHHHHENLYFQGNPPAPADAEVPSKWQAAQGKALTVRLEPSKNKQEAVSDVVITYKPDVVLAYGRAKFHLPKGFSAVAGDLINGQPLTADYIQNGGQTVTLPFGVDIGAMRIFELRLVGKKLPSAGQYKFRAEYWGIGIGIYNTAEAVLELRN

SEQ ID NO: 27 - DNA, 417 bp, Artificial Sequence
  Sequence optimized synthetic gene
atgcatcatcaccatcaccacgaaaacctgtattttcagggcaccttctaccgtaacgcgccgaccctgcacatcgagaccgttgacagcaacaaggaatggaccaccagcgatattgaggtgacctataaaccgaacttctttgttggtagcagctacgtggaattcaactttccgtatcgttttcacgcgaacacccgtgacagcctgaacggtcgtaccctgaactacacccagatcctgaacgatggccaaaccgtgcgtgttccggtgtatgcgttcagcagcagcgagtttaagctggttatggtgcgtaaaaccctgccgaacgcgggtacccaccgtatcaccgcggaactgcagaagttcggccgtaactacaaccacgcggaggcgaccgttgacattctgccgcgt

SEQ ID NO: 28 - protein, 139 aa, Bacillus licheniformis
MHHHHHHENLYFQGTFYRNAPTLHIETVDSNKEWTTSDIEVTYKPNFFVGSSYVEFNFPYRFHANTRDSLNGRTLNYTQILNDGQTVRVPVYAFSSSEFKLVMVRKTLPNAGTHRITAELQKFGRNYNHAEATVDILPR

SEQ ID NO: 29 - DNA, 417 bp, Artificial Sequence
  Sequence optimized synthetic gene
cgtacccaaagcaccgcgagcctgtttgcgaccatcaccggcgcgagcaagaccgagtggagcttcagcgacattgaactgacctatcgtccgaacaccctgctgagcctgggtgtgatggagttcaccctgccgagcggctttaccgcgaacaccaaggacaccctgaacggtaacgcgctgcgtaccacccagatcctgaacaacggcaaaaccgtgcgtgttccgctggcgctggatctgctgggtgcgggtgaatttaagctgaaactgaacaacaagaccctgccggcggcgggtacctacaccttccgtgcggagaacaagagcctgagcatcggcaacaaattttatgcggaagcgagcattgatgttgcgaagcgtagcaccccgccgacccaaccgtgcggttgcaac

SEQ ID NO: 30 - protein, 153 aa, Bacillus subtilis
MHHHHHHENLYFQGRTQSTASLFATITGASKTEWSFSDIELTYRPNTLLSLGVMEFTLPSGFTANTKDTLNGNALRTTQILNNGKTVRVPLALDLLGAGEFKLKLNNKTLPAAGTYTFRAENKSLSIGNKFYAEASIDVAKRSTPPTQPCGCN

SEQ ID NO: 31 - DNA, 366 bp, Artificial Sequence
  Sequence optimized synthetic gene
gcggtgatcgaggcgaagaccgttaacagcaccaaagagtacgcgaccagcgacattgaagtgacctataaaccgaacgcgctgctggcggtgggtgcggttgagttccagtttccggacggtttcaacgcgaccgtgcgtgatagcgttaacggccgtaccctgaaggaaacccaaatcctgaactacggtaaaaccgttcgtctgccgctgaccctggacctgttcggtgcgagcgagtataacctggtgctggttcgtaagaacctgccgcgtgcgggcacctacaccatcaagggtgactatgtgaacggtctgggtgcgggtagcctgtatgcggaaaccaaactggttattgatccgcgt

SEQ ID NO: 32 - protein, 136 aa, Bacillus venelenzis
MHHHHHHENLYFQGAVIEAKTVNSTKEYATSDIEVTYKPNALLAVGAVEFQFPDGFNATVRDSVNGRTLKETQILNYGKTVRLPLTLDLFGASEYNLVLVRKNLPRAGTYTIKGDYVNGLGAGSLYAETKLVIDPR

SEQ ID NO: 33 - protein, 41 aa, Artificial Sequence
  Consensus amino acid sequence
SPGVLSGNVVQVPVDVPVNVCGNTVNVVGLLNPAFGNSCVN

SEQ ID NO: 34 - protein, 252 aa, Streptomyces_coelicolor_A3(2)
MVAAAAATGILSLCGSPALADSHADGAATNSPGAVSGNALQVPVDVPVNACGNTVDVIAALNPAFGNECENASDEKTDGHGGGYGEDASSSSSSSTSASSSGSHADGATEGSPGVGSGNNAQVPVDVPVNLCGNTVDVIAALNPVFGNKCENDAEEPPGYGEEEPPPPTTPPGYGEEEPPPPTHEEPPPPSGEEEPPPPSEEEHTPPAPQTEQPPALAETGSEGTLGAAAAGAVLIAGGAILYRRGRALSGR

SEQ ID NO: 35 - protein, 237 aa, Streptomyces_coelicolor_A3(2)
MRRVTRNGVLAVAASGALAVTMPAYAAFASDGAGAEGSAAGSPGLISGNTVQLPVDVPVDVCGNTVNVVGLLNPAAGNGCADSGEPGASYQAAGASGGTSGSATEATSGGAAAEGSGKDSPGVLSGNGVQLPVHLPVNVSGNSVNVVGIGNPAVGNESTNDSGDHPEPVRPPAEPEPSAPEEERAGPGPSAHAAPPREEVSLAHTGTDRTLPTLAGGAALVLGGTVLYRRFRPGSGD

SEQ ID NO: 36 - protein, 259 aa, Streptomyces_coelicolor_A3(2)
MRQATRKGLMTMAAATGVIAAAGGAAHADSGAHGTSSGSPGVLSGNTVQAPVHVPVNVCGNTVDVVGVLNPAMGNACANQGGGASGGHGGHGGHGGYGDSGGEGGSHGGSHAGGHATDSPGVGSGNHVEVPIDVPVNVCGNSIDVVGALNPTTGNDCGNGGGGDHSTPPGDHETPPGEPHNPGNPGNPDTPDKPSGPDDETPGDSTDGNRPGAQTVDQPRGDAALAETGSDLPLGLALPVGAGALLAGTVLYRKARASV

SEQ ID NO: 37 - protein, 74 aa, Streptomyces_coelicolor_A3(2)
MKKSAAVVAGAIMALGMAAPAFADAGAEGAAVGSPGVLSGNVIQVPVHVPVNVCGNSINVVGLLNPAFGNKCEN

SEQ ID NO: 38 - protein, 82 aa, Streptomyces_coelicolor_A3(2)
MKNLKKAAAVTMVAGGLIAAGAGMASATDGGAHAHGKAVGSPGVASGNLVQAPIHIPVNAVGNSVNVIGVLNPAFGNLGVNH

SEQ ID NO: 39 - protein, 88 aa, Streptomyces_coelicolor_A3(2)
MYNPKEHFSMSRIAKGLALTSVAAAAVAGTAGVAAADSGAQAAAAHSPGVLSGNVVQVPVHIPVNVCGNTIDVIGLLNPAFGNECEND

SEQ ID NO: 40 - protein, 90 aa, Streptomyces_coelicolor_A3(2)
MSRIAKAAGVALGTGAVVLSGTGMAMADAGAAGAAVGSPGVLSGNVVQVPVHVPVNLCGNTIDVIGLLNPAFGNACENGDDDDKSGGYGG

SEQ ID NO: 41 - protein, 77 aa, Streptomyces_coelicolor_A3(2)
MLKKVVAAAAATGGLVLAGAGMAVADSGAQGAAVHSPGVLSGNVVQVPVHVPVNVCGNTISVIGLLNPAFGNVCINK

SEQ ID NO: 42 - protein, 79 aa, Catenulispora_acidiphila_DSM_44928]
MLKTKKIAALVAATGGLVMAGAGMASAEATAGGSSVGSPGIVSGNTIQVPVHVPVNACGLTVSVIGILDQAFGNTCVNG

SEQ ID NO: 43 - protein, 79 aa, Catenulispora_acidiphila
MSMRKTLVAAAFAAVAVLGTAGTASASGAGAIGGAIGSPGLLSGNNIQIPINIPINLCGNDISVLAALTGAAGNTCVNY

SEQ ID NO: 44 - protein, 75 aa, Streptomyces coelicolor
MKKSAAVVAGAIMALGMAAPAFADAGAEGAAVGSPGVLSGNVIQVPVHVPVNVCGNSINVVGLLNPAFGNKCEND

SEQ ID NO: 45 - protein, 89 aa, Streptomyces_pristinaespiralis
MSRIAKAAAVVAGTGAILAGGAGMAAADAGAHGGAAGSPGVVSGNAVQVPVHVPVNVCGNTVNVIALLNPTFGNQCANVDGGDHGAYGG

SEQ ID NO: 46 - protein, 340 aa, Catenulispora_acidiphila_DSM_44928
MQSQVKRRIVFGLTTGGMLATGGVGLAHADAAAAGVGDGATTAGSPGILSGNTIQIPVNIPINVCGVTANVVGLLNPAEGNHCANSGGATANGGGPSSGGASASGSSVGSPGILSGNTIQAPVRVPVNACGDTVNVVGVGNGAKGNHCANEGGTTGGGATATGSSVGSPGIISGNTVQVPVNVPVNLCGDTVNVVGVGNNADGNHCLNAGGGAVTGGSTATGSSVGSPGIVSGNTIQLPISIPVNVCGDSVNVVGIANGAAGNACANDTPAPPTVTPPPTTTTGWTAPRTAPTETGTAVPAATGMLAHTGADGLMLAPLGAALMGGGAFMYRKYKPRRMF

SEQ ID NO: 47 - protein, 267 aa, Catenulispora_acidiphila
MLNPAEGNHCANSGGATANGGGPSSGGASASGSSVGSPGILSGNTIQAPVRVPVNACGDTVNVVGVGNGAKGNHCANEGGTTGGGATATGSSVGSPGIISGNTVQVPVNVPVNLCGDTVNVVGVGNNADGNHCLNAGGGAVTGGSTATGSSVGSPGIVSGNTIQLPISIPVNVCGDSVNVVGIANGAAGNACANDTPAPPTVTPPPTTTTGWTAPRTAPTETGTAVPAATGMLAHTGADGLMLAPLGAALMGGGAFMYRKYKPRRMF

SEQ ID NO: 48 - protein, 52 aa, Streptomyces coelicolor_A3(2)
DAGAEGAAVGSPGVLSGNVIQVPVHVPVNVCGNSINVVGLLNPAFGNKCEND

SEQ ID NO: 49 - protein, 55 aa, Streptomyces coelicolor_A3(2)
TDGGAHAHGKAVGSPGVASGNLVQAPIHIPVNAVGNSVNVIGVLNPAFGNLGVNH

SEQ ID NO: 50 - protein, 52 aa, Streptomyces coelicolor_A3(2)
DSGAQAAAAHSPGVLSGNVVQVPVHIPVNVCGNTIDVIGLLNPAFGNECEND

SEQ ID NO: 51 - protein, 63 aa, Streptomyces coelicolor_A3(2)
DAGAAGAAVGSPGVLSGNVVQVPVHVPVNLCGNTIDVIGLLNPAFGNACENGDDDDKSGGYGG

SEQ ID NO: 52 - protein, 52 aa, Streptomyces coelicolor_A3(2)
DSGAQGAAVHSPGVLSGNVVQVPVHVPVNVCGNTISVIGLLNPAFGNVCINK

SEQ ID NO: 53 - protein, 104 aa, Conidiobolus coronatus ATCC 28846
MQLLILASLIAFIASAPVANGGDFKSHGHIAGNNIRGSVHAPQNTCNNGVKVIGGTNTSFDNSCTNTSVESDTDDHSNSDTDDHSNSDTDDHSNSEDESGSDDS

SEQ ID NO: 54 - DNA, 156 bp, Artificial Sequence
  Sequence optimized synthetic gene
gatagtggtgctcaagcggcggctgcccatagtccgggtgtcctgtctggtaatgtggttcaagttccggttcatattccggttaatgtgtgcggcaatacgattgatgtgattggtctgctgaacccggccttcggtaatgaatgtgaaaatgat

SEQ ID NO: 55 - protein, 456 aa, Streptomyces coelicolor)A3(2)
MGSSHHHHHHGTKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTGTDYDIPTTKLGSGSSGSGENLYFQGDSGAQAAAAHSPGVLSGNVVQVPVHIPVNVCGNTIDVIGLLNPAFGNECEND

SEQ ID NO: 56 - protein, 96 aa, Engystomops pustulosus
MLILDGDLLKDKLKLPVIDNLFGKELLDKFQDDIKDKYGVDTKDLKILKTSEDKRFYYVSVDAGDGEKCKFKIRKDVDVPKMVGRKCRKDDDDDDG

SEQ ID NO: 57 - protein, 217 aa, Leptodactylus vastus
LLEGFLVGGGVPGPGTACLTKALKDSGDLLVELAVIICAYQNGKDLQEQDFKELKELLERTLERAGCALDDIVADLGLEELLGSIGVSTGDIIQGLYKLLKELKIDETVFNAVCDVTKKMLDNKCLPKILQGDLVKFLKDLKYKVCIEGGDPELIIKDLKIILERLPCVLGGVGLDDLFKNIFVKDGILSFEGIAKPLGDLLILVLCPNVKNINVSS

SEQ ID NO: 58 - protein, 218 aa, Leptodactylus fuscu
RISYWVGAKPILIHALKNTSALILQLHAMICIYQNGTGNLTDADFQKLNILVNNTLSKYGCTLDMLMKELGIGSLNLQDLLGGLSPAVGDTFKIIYELLKGVQLEKTVLTTLCDAIGKLINEGCLPDLFQPDLVEFFKNFKKLCESFNSGDLDAFTKSLEKVLERMKCLLHGLSITDILNNISAQDGKLIFSGLAKPLGDLLTTLCPLIGKLAPATGD

SEQ ID NO: 59 - protein, 164 aa, Bufo gargarizans
MKTILLLVLAGLSVSYGFNCIQRYQAATTLQCLTNILEIAPDFLEKLVYFLCNYDEGMKNNKGEFEAMFRDLIEILECAGCALDEITGTNISIEELLGDIGGAGRQALFGILKAADGLQLTGTVADLGCSLLKGLGTPGGLINLNGVLGGLTGGLGGILSLVKN

SEQ ID NO: 60 - protein, 135 aa, Engystomops pustulosus
MAAIQFALFFVFAVISQHCAYGFLPLGGGNIGGGAKLGPEKPATPGIQDLLKSLLSVLNLSPPAIPEDAEAVSYRDAKNGKFRLIKIHLGGELYCHVKQIAGPILALPIVSDVVEVTGKECGKTEDDPLEDFPIP

SEQ ID NO: 61 - protein, 174 aa, Engystomops pustulosus
MIDPTGLVQILLLEQVVHKIPPGNINLARTGIATQDSDYTASAVPSEARLAIDGNRNSDFNQKSCSHTGGNEPAWWRLELKKKSKISVVVIAIRSDCCMDRFKGAELRIGNSQDATVNPICGKVSAVKGSNYLFCCDGMEGKYISVVIPDRHEFLSLCEVEVYGAKPIEGTHCK

SEQ ID NO: 62 - protein, 193 aa, Engystomops pustulosus
MKLLLLVVLVWTASDEASADRNLALDGRATMSSIWMDPDIRQSFLGVAMNGIDGNTDSVYFHGSCFHTGLDSPAWYRVDLLRTSKISSITITNRGDFGSRTNGAEIRIGDSLANNGNNNPRCALVTSIADGETRTFQCNNMVGRYVNIVLTGKTEFLHLCEVQIFGENLPRSFSCQYSNDGMITLLVSTRFMK

SEQ ID NO: 63 - protein, 178 aa, Engystomops pustulosus
MGAPGGAAGPLLVLNILGSVVHETKPPEGVNLALKGIASSDSIASNGSVTGLAAKAIDGIRVSDFFKGHCSLTNGLNNPTWWKVDLKKSYKISSVFVTNRDDCCTERLLHAEIRIGSNPDHNHNPICAEVKTVASSNIGFCCGGMEGRYVSVSVPRKEQLSLCEVEVYGDLKKVLHCA

SEQ ID NO: 64 - protein, 246 aa, Engystomops pustulosus
MILILGVLLLGAEASAETLCIPGRMKQLDAGAGRVVAVKSNGDVYQLLENNWVQIPGKLIHVTVGPAGLWGVNKDKNIYKYVDNDWLQVDGLLNQIDAGGNRFVVGVNDNEDIFCLNQDQTTSNAVKLDYKGVDGKLKYYSSGGYGSWGVNAAYDIFYRRNVHPMSCQGTNWENVEGKLVMLEVAEDGSVYGVNYNGHVYKREGITAGNPMGTSWTYLKVDEKVRHVSYDRGVLYVVTIDDRIFRC

SEQ ID NO: 65 - protein, 228 aa, Equus caballus
MLKVSCLFVLLCGLLVPSSAQQIPPEVSSQITDALTQGLLDGNFLSLLNAINLEGLLNTILDQVTGLLNILVGPLLGPSDAEIKLQDTRLLQLSLEFSPDSKGIDIWIPLELSVYLKLLILEPLTLYVRTDIRVQLRLESDEDGKYRLAFGHCSLLPRAIELQSGNPLSLPVNAVLGTIENALGNFITEDLGAGLCPTLNSLVSNLDLQLVNNLINLILDRANVDLSV

SEQ ID NO: 66 - DNA, 290 bp, Artificial Sequence
  Sequence optimized synthetic gene
atgctgatcctggacggtgatctgctgaaggacaaactgaagctgccggtgattgataacctgttcggtaagagctgctggacaagtttcaggacgatatcaaggataagtacggcgttgacaccaaggatctgaaaattctgaagaccagcgaggacaaacgtttctactatgtgagcgttgacgcgggtgatggcgaaaaatgcaagtttaaaatccgtaaagacgtggatgttccgaagatggtgggtcgtaaatgccgtaaggacgatgacgatgacgatggctat

SEQ ID NO: 67 - protein, 111 aa, Engystomops pustulosus
MHHHHHHENLYFQGMLILDGDLLKDKLKLPVIDNLFGKELLDKFQDDIKDKYGVDTKDLKILKTSEDKRFYYVSVDAGDGEKCKFKIRKDVDVPKMVGRKCRKDDDDDDGY

SEQ ID NO: 68 - DNA, 630 bp, Artificial Sequence
  Sequence optimized synthetic gene
ttgttagaaggtttcttggttggtggtggtgttccaggtccaggtactgcttgtttgacaaaagcattaaaagattctggtgacttgttggttgaattggctgttattatctgtgcataccaaaacggtaaagatttgcaagaacaagatttcaaggaattgaaggaattgttggaaagaactttagaaagagctggttgtgcattggatgatattgttgctgatttgggtttagaagaattgttaggttctattggtgtttcaacaggtgacattattcaaggtttgtacaagttgttgaaggaattgaagattgatgaaactgtttttaatgcagtttgtgatgttactaagaaaatgttggataataagtgtttgccaaagatcttgcagggtgacttggttaagttcttgaaggatttgaagtacaaggtttgtattgaaggtggtgacccagaattgattattaaggatttgaagatcatcttggaaagattaccatgtgttttgggtggtgttggtttggatgatttgtttaaaaacatcttcgttaaggatggtatcttgtcattcgaaggtatcgctaaaccattgggtgacttgttaattttggttttatgtccaaatgtt

SEQ ID NO: 69 - protein, 230 aa, Leptodactylus vastus
HHHHHHENLYFQGLLEGFLVGGGVPGPGTACLTKALKDSGDLLVELAVIICAYQNGKDLQEQDFKELKELLERTLERAGCALDDIVADLGLEELLGSIGVSTGDIIQGLYKLLKELKIDETVFNAVCDVTKKMLDNKCLPKILQGDLVKFLKDLKYKVCIEGGDPELIIKDLKIILERLPCVLGGVGLDDLFKNIFVKDGILSFEGIAKPLGDLLILVLCPNVKNINVSS

SEQ ID NO: 70 - protein, 71 aa, Trichoderma reesei
AVCPTGLFSNPLCCATNVLDLIGVDCKTPTIAVDTGAIFQAHCASKGSKPLCCVAPVADQALLCQKAIGTF